(19) United States
(12) Patent Application Publication
(10) Pub. No.: US 2023/0241201 A1
(43) Pub. Date: Aug. 3, 2023

(54) CORONAVIRUS VACCINES

(71) Applicant: KATHOLIEKE UNIVERSITEIT LEUVEN, Leuven (BE)

(72) Inventors: Kai DALLMEIER, Kessel-Lo (BE); Johan NEYTS, Kessel-Lo (BE); Lorena SANCHEZ FELIPE, Wijgmaal (BE); Hendrik, Jan THIBAUT, Heverlee (BE); Dominique VAN LOOVEREN, Heist-op-den-Berg (BE); Thomas VERCRUYSSE, Wilsele (BE)

(21) Appl. No.: 17/802,001

(22) PCT Filed: Mar. 1, 2021
(86) PCT No.: PCT/EP2021/055013

§ 371 Date: Aug. 24, 2022

(30) Foreign Application Priority Data
Feb. 27, 2020 (GB) 2002766.0
Jul. 8, 2020 (GB) 2010479.0
Sep. 4, 2020 (GB) 2013912.7

(51) Int. Cl. A61K 39/215; A61P 31/14; A61K 39/12; C12N 15/86

(52) U.S. Cl. CPC A61K 39/215; A61P 31/14; A61K 39/12; C12N 15/86; A61K 2039/5254

(57) ABSTRACT

The invention relates to polynucleotides comprising a sequence of a live, infectious, attenuated Flavivirus wherein a nucleotide sequence encoding the S1 and S2 subunits of a coronavirus Spike protein is located, such that a chimeric virus is expressed.

### CROSS-REFERENCE TO RELATED APPLICATION(S)

This present application is a national stage application of International Patent Application No. PCT/EP2021/055013, filed Mar. 1, 2021, which claims priority to British Patent Application No. 2002766.0, filed Feb. 27, 2020, British Patent Application No. 2010479.0, filed Jul. 8, 2020, and British Patent Application No. 2013912.7, filed Sep. 4, 2020, the disclosures of which are hereby incorporated by reference in their entireties.

### FIELD OF THE INVENTION

The present invention relates to chimeric flaviviruses comprising one or more antigen(s), and DNA vaccines thereof.

### BACKGROUND OF THE INVENTION

Since it discovery in December 2019, a novel coronavirus, now known as Severe Acute Respiratory Syndrome Coronavirus-2 (SARS-CoV2), is infecting people in all continents. The sequence of the novel SARS-CoV2 has been meanwhile sequenced.

Protective immunity against SARS-CoV-2 and other coronaviruses is believed to depend on neutralizing antibodies (NAbs) that target the viral spike (S) protein. In particular, NAbs specific for the N-terminal S1 domain—which contains the angiotensin-converting enzyme 2 (ACE2) receptor-binding domain—have previously been shown to prevent viral infection in several animal models.

The yellow fever 17D (YF17D) is used as a vector in two human vaccines. The Imojev vaccine is a recombinant chimeric virus vaccine developed by replacing the cDNA encoding the envelope proteins of YF17D with that of an attenuated Japanese encephalitis virus (JEV) strain SA14-14.2. The Dengvaxia vaccine is a live-attenuated tetravalent chimeric made by replacing the pre-membrane and envelope structural genes of YF17D strain vaccine with those from the Dengue virus 1, 2, 3 and 4 serotypes.

International patent application WO2014174078 describes a bacterial artificial chromosome (BAC) comprising the cDNA of a YFV-17D vaccine, wherein cDNAs encoding for heterologous proteins can be inserted in the cDNA YFV-17D within the BAC such as between E and NS1 genes, in the C gene or in the untranslated regions of the YFV-17D cDNA.

International patent application WO2019068877 describes polynucleotides, such as a BAC, comprising the sequence of a flavivirus preceded by a sequence encoding an N terminal part of a flavivirus Capsid protein, an immunogenic protein, or a part thereof comprising a an immunogenic peptide, and a 2A cleaving peptide.

There is a growing need for prophylactic or therapeutic vaccines against the SARS-CoV2 virus.

### SUMMARY OF THE INVENTION

The explosively expanding COVID-19 pandemic urges the development of safe, efficacious and fast-acting vaccines to quench the unrestrained spread of SARS-CoV-2. Several promising vaccine platforms, developed in recent years, are leveraged for a rapid emergency response to COVID-19.

Present inventors are the first to find that large antigens can be expressed in an efficacious way as part of a polynucleotide comprising a sequence of a live, infectious, attenuated flavivirus, such as YF17D, and that such chimeric virus is sufficiently stable to be used for vaccination purposes. Accordingly, the present invention provides effective vaccines based on live, infectious, attenuated flavivirus, such as YF17D comprising a large antigen, such as a spike protein of a coronavirus.

Present inventors have moreover found that a polynucleotide comprising a nucleotide sequence of a live, infectious, attenuated Flavivirus, such as YF17D, wherein a nucleotide sequence encoding both the S1 and S2 unit of a coronavirus Spike protein is inserted (i.e. located) ensures an effective and stable vaccine against said coronavirus. Such vaccines, and in particular vaccines encoding the non-cleavable form of coronavirus spike protein, allow to obtain an unexpectedly high immunogenicity and efficacy in vivo with only a single dose. Furthermore, such vaccines also have an excellent safety profile.

For example, present inventors employed the live-attenuated YF17D vaccine as a vector to express the prefusion form of the SARS-CoV-2 Spike antigen. In mice, the vaccine candidate comprising a nucleotide sequence encoding the S1 and S2 subunit of the coronavirus Spike protein, wherein the S1/2 cleavage site is mutated to prevent proteolytic processing of the S protein in the S1 and S2 subunits, also referred to in the present specification as “YF-S0” or “construct 2”, induces high levels of SARS-CoV-2 neutralizing antibodies and a favorable Th1 cell-mediated immune response. In a stringent hamster SARS-CoV-2 challenge model, vaccine candidate YF-S0 prevents infection with SARS-CoV-2. Moreover, a single dose confers protection from lung disease in most vaccinated animals even within 10 days. More particularly, the vaccination of macaques with a relatively low subcutaneous dose of YF-S0 led to rapid seroconversion tot high Nab titres. These results indicate that at least YF-S0 is a potent SARS-CoV-2 vaccine candidate.

A first aspect provides a polynucleotide comprising a nucleotide sequence of a live, infectious, attenuated Flavivirus wherein a nucleotide sequence encoding the S1 and S2 subunit of a coronavirus Spike protein is located, so as to allow expression of a chimeric virus from said polynucleotide.

In particular embodiments, the nucleotide sequence encoding the S1/S2 cleavage site is mutated, thereby preventing proteolytic processing of S protein in the S1 and S2 subunits.

In particular embodiments, the nucleotide sequence encoding the S1 and S2 subunit of the coronavirus Spike protein is located 3′ of the nucleotide sequences encoding the envelope protein of the flavivirus and 5′ of the nucleotide sequences encoding the NS1 protein of the flavivirus.

In particular embodiments, the nucleotide sequence encoding the S1 and S2 subunit of the coronavirus Spike protein does not comprise the nucleotide sequence encoding the signal peptide or part of the signal peptide of the coronavirus Spike protein, preferably wherein the nucleotide sequence encoding at least the S2 subunit of a coronavirus Spike protein does not comprise the first 39 nucleotides of the nucleotide sequence encoding the signal peptide of the coronavirus Spike protein.

In particular embodiments, a nucleotide sequence encoding a transmembrane (TM) domain of a further flavivirus is located 3′ of the nucleotide sequence encoding the S1 and S2 subunit of the coronavirus Spike protein, and 5′ of the NS1 region of the NS1-NS5 region, preferably wherein the TM domain of a further flavivirus is a West Nile virus transmembrane domain 2 (WNV-TM2).

In particular embodiments, the polynucleotide comprises 5′ to the nucleotide sequence encoding the S1 and S2 subunit of the coronavirus Spike protein, a sequence encoding an NS1 signal peptide.

In particular embodiments, the nucleotide sequence encoding the S2′ cleavage site is mutated, thereby preventing proteolytic processing of the S2 unit.

In particular embodiments, the coronavirus is severe acute respiratory syndrome coronavirus-2 (SARS-CoV-2).

In particular embodiments, the Flavivirus is yellow fever virus.

In particular embodiments, the Flavivirus is yellow fever 17 D (YF17D) virus.

In particular embodiments, the polynucleotide comprises a sequence selected from the group consisting of SEQ ID NO: 3, SEQ ID NO: 5, and SEQ ID NO: 7, preferably comprising a sequence as defined by SEQ ID NO: 5.

In particular embodiments, the polynucleotide is a bacterial artificial chromosome (BAC).

A further aspect provides a chimeric live, infectious, attenuated Flavivirus encoded by a polynucleotide as taught herein.

A further aspect provides a pharmaceutical composition comprising the polynucleotide as taught herein or the chimeric virus as taught herein, and a pharmaceutically acceptable carrier, preferably wherein the pharmaceutical composition is a vaccine.

A further aspect provides a polynucleotide as taught herein, a chimeric virus as taught herein, or a pharmaceutical composition as taught herein for use as a medicament, preferably wherein the medicament is a vaccine.

A further aspect provides a polynucleotide as taught herein, a chimeric virus as taught herein, or a pharmaceutical composition as taught herein for use in preventing a coronavirus infection, preferably a SARS-CoV-2 infection.

A further aspect provides an in vitro method of preparing a vaccine against a coronavirus infection, comprising the steps of:

a) providing a BAC which comprises:
an inducible bacterial ori sequence for amplification of said BAC to more than 10 copies per bacterial cell, and
a viral expression cassette comprising a cDNA of a chimeric virus comprising a polynucleotide as taught herein, and comprising cis-regulatory elements for transcription of said viral cDNA in mammalian cells and for processing of the transcribed RNA into infectious RNA virus,
b) transfecting mammalian cells with the BAC of step a) and passaging the infected cells,
c) validating replicated virus of the transfected cells of step b) for virulence and the capacity of generating antibodies and inducing protection against coronavirus infection,
d) cloning the virus validated in step c) into a vector, and formulating the vector into a vaccine formulation.
In particular embodiments, the vector is BAC, which comprises an inducible bacterial ori sequence for amplification of said BAC to more than 10 copies per bacterial cell.

### BRIEF DESCRIPTION OF DRAWINGS

FIG. 1. Vaccine design and antigenicity. (A) Schematic representation of YF17D-based SARS-CoV-2 vaccine candidates (YF-S). YF-S1/2 expresses the native cleavable post-fusion form of the S protein (S1/2), YF-S0 the non-cleavable pre-fusion conformation (S0), and YF-S1 the N-terminal (receptor binding domain) containing S1 subunit of the S protein. For molecular details in vaccine design see Methods section. (B) Representative pictures of plaque phenotypes from different YF-S vaccine constructs on BHK-21 cells in comparison to YF17D. (C) Confocal immunofluorescent images of BHK-21 cells three days post-infection with different YF-S vaccine constructs staining for SARS-CoV-2 Spike antigen and YF17D (white scalebar: 25 μm). (D) Immunoblot analysis of SARS-CoV-2 Spike (S1/2, S0 and S1) antigen and SARS Spike expression after transduction of BHK-21 cells with different YF-S vaccine candidates. Prior to analysis, cell lysates were treated with Peptide-N-glycosidase F (PNGase F) to remove their glycosylation or left untreated (black arrows—glycosylated forms of S; white arrows—de-glycosylated forms).

FIG. 2. Attenuation of YF-S vaccine candidates. (A) Survival curve of suckling Balb/c mice (up to 21 days) after intracranial (i.c.) inoculation with 100 plaque-forming-unit (PFU) of vaccine candidates YF-S1/2 (n=8), YF-S0 (n=8), YF-S1 (n=8) in comparison to sham (n=10) or YF17D (n=9). (B) Survival curve of AG129 mice (up to 21 days) after intraperitoneal (i.p.) inoculation with a dose range of YF-S0 (10², 10³ and 10⁴ PFU) in comparison to YF17D (1, 10 and 10² PFU black and grey). Statistical significance between groups was calculated by the Log-rank Mantel-Cox test (**** P<0.0001).

FIG. 3. Immunogenicity and protective efficacy of YF-S vaccine candidates in hamsters. (A) Schematic representation of vaccination and challenge schedule. Syrian hamsters were immunized twice i.p. at day 0 and 7 with 10³ PFU each of vaccine constructs YF-S1/2 (n=12), YF-S0 (n=12), YF-S1 (n=12), sham (white, n=12) or YF17D (grey, n=12) (two independent experiments). Subsequently, animals were intranasally inoculated with 2×10⁵ tissue culture infective dose (TCID₅₀) of SARS-CoV-2 and followed up for four days. (B-D) Humoral immune responses. Neutralizing antibodies (nAb) (B) and total binding IgG (bAb) (C) in hamsters vaccinated with different vaccine candidates (sera collected at day 21 post-vaccination in both experiments; minipools of sera of three animals each were analyzed for quantification of bAb; minipools of sera of three animals each were analyzed for quantification of bAb). (D) Seroconversion rates at indicated days post-vaccination with YF-S1/2 and YF-S0 (number of animals with detectable bAbs at each time point are referenced). (E, F) Protection from SARS-CoV-2 infection. Viral loads in lungs of hamsters four days after intranasal infection were quantified by RT-qPCR (E) and virus titration (F). Viral RNA levels were determined in the lungs, normalized against β-actin and fold-changes were calculated using the 2^{-ΔΔCq)} method compared to the median of sham-vaccinated hamsters. Infectious viral loads in the lungs are expressed as number of infectious virus particles per 100 mg of lung tissue. (G) Anamnestic response. Comparison of the levels of nAbs prior and four days after challenge. For a pairwise comparison of responses in individual animals see FIG. 11C and D. Dotted line indicating lower limit of quantification (LLOQ) or lower limit of detection (LLOD) as indicated. Data shown are medians±interquartile range (IQR). Statistical significance between groups was calculated by the non-parametric ANOVA, Kruskall-Wallis with uncorrected Dunn's test (B-F), or a non-parametric two-tailed Wilcoxon matched-pairs rank test (G) (ns=Non-Significant, P>0.05, *P<0.05, ** P<0.01, *** P<0.001, **** P<0.0001).

FIG. 4. Protection from lung disease in YF-S vaccinated hamsters. (A) Representative hematoxylin and eosin (H&E) images of the lungs of a diseased (sham-vaccinated and infected) and a YF-S0-vaccinated and challenged hamster. Peri-vascular edema (arrow B); peri-bronchial inflammation (arrows R); peri-vascular inflammation (arrow G); bronchopneumonia (circle), apoptotic body in bronchial wall (arrowhead R). (B) A spider-web plot showing histopathological score for signs of lung damage (peri-vascular edema, bronchopneumonia, peri-vascular inflammation, peri-bronchial inflammation, vasculitis, intra-alveolar hemorrhage and apoptotic bodies in bronchus walls) normalized to sham (grey). Black scalebar: 100 μm (C-D) Micro-CT-derived analysis of lung disease. Five transverse cross sections at different positions in the lung were selected for each animal and scored to quantify lung consolidations (C) or used to quantify the non-aerated lung volume (NALV) (D), as functional biomarker reflecting lung consolidation. (E) Heat-map showing differential expression of selected antiviral, pro-inflammatory and cytokine genes in lungs of sham- or YF-S-vaccinated hamsters after SARS-CoV-2 challenge four days p.i. (n=12 per treatment group) relative to non-treated non-infected controls (n=4) (scale represents fold-change over controls). RNA levels were determined by RT-qPCR on lung extracts, normalized for β-actin mRNA levels and fold-changes over the median of uninfected controls were calculated using the 2^{(ΔΔCq)} method. (F) Individual expression profiles of mRNA levels of interleukin-6 (IL-6), IP-10, interferon lambda (IFN-λ) and MX2, with data presented as median±IQR relative to the median of non-treated non-infected controls. For IFN-k, where all control animals had undetectable RNA levels, fold-changes were calculated over the lowest detectable value (LLOD—lower limit of detection; dotted line). Statistical significance between conditions was calculated by the non-parametric ANOVA, Kruskall-Wallis with uncorrected Dunn's test (ns=Not-Significant, P>0.05, * P<0.05, ** P<0.01, *** P<0.001).

FIG. 5. Humoral immune response elicited by YF-S vaccine candidates in mice. (A) Schematic presentation of immunization and challenge schedule. Ifnar^{-/-} mice were vaccinated twice i.p. with 400 PFU each at day 0 and 7 in five groups: constructs YF-S1/2 (n=11), YF-S0 (n=11), YF-S1 (n=13), sham (white, n=9) or YF17D (grey, n=9). (B, C) SARS-CoV-2 specific antibody levels. Titers of nAbs (B) and bAbs (C) at day 21 post-vaccination. minipools of sera of three animals each were analyzed for quantification of bAb). (D) Seroconversion rates. Rates at indicated days post-vaccination with YF-S1/2 and YF-S0 (number of animals with detectable bAbs at each time point are referenced). For quantification of bAbs, minipools of sera of two to three animals each were analyzed. Dotted lines indicate lower limit of quantification (LLOQ) or lower limit of detection (LLOD). (E) IgG for YF-S1/2 and YF-S0. Ratios of IgG2b or IgG2c over IgG1 (determined for minipools of two to three animals each) plotted and compared to a theoretical limit between Th1 and Th2 response (dotted line indicates IgG2b/c over IgG1 ratio of 1). Data shown are medians±IQR from three independent vaccination experiments (n>9 for each condition). Statistical significance between groups was calculated by a non-parametric ANOVA, Kruskall-Wallis with uncorrected Dunn's test (B-C) or parametric One-Sample T-test (D) (ns=Not-Significant, P>0.05, * P<0.05, ** P<0.01, *** P<0.001, **** P<0.0001).

FIG. 6. Cell-mediated immune (CMI) responses of YF-S vaccine candidates in mice. Spike-specific T-cell responses were analyzed by ELISpot and intracellular cytokine staining (ICS) of splenocytes isolated from ifnar^{-/-} mice 21 days post-prime (i.e., two weeks post-boost) immunization with YF-S1/2, YF-S0, YF-S1 in comparison to sham (white) or YF17D (grey). (A) Quantitative assessment of SARS-CoV-2 specific CMI response by ELISpot. Spot counts for IFN-γ-secreting cells per 10⁶ splenocytes after stimulation with SARS-CoV-2 Spike peptide pool. (B) Transcriptional profile induced by YF-S vaccination. mRNA expression levels of transcription factors (TBX21, GATA3, RAR-related orphan receptor C (RORC), forkhead box protein P3 (FOXP3)) determined by RT-qPCR analysis of Spike peptide-stimulated splenocytes (n=5-7 per condition). Data were normalized for glyceraldehyde 3-phosphate dehydrogenase (GAPDH) mRNA levels and fold-changes over median of uninfected controls were calculated using the 2^{(ΔΔCq)} method. (C-F) Percentage of IFN-γ (C) and tumor necrosis factor alpha (TNF-α) (D) expressing CD8, and IFN-γ expressing CD4 (E) and γ/δ (F) T-cells after stimulation with SARS-CoV-2 Spike peptide pool. All values normalized by subtracting spots/percentage of positive cells in corresponding unstimulated control samples. Data shown are medians±IQR. Statistical significance between groups was calculated by the non-parametric ANOVA, Kruskall-Wallis with uncorrected Dunn's test (ns=Not-Significant, P>0.05, * P<0.05, ** P<0.01, *** P<0.001). (G, H) Profiling of CD8 T-cells from YF-S1/2 and YF-S0 vaccinated mice by t-SNE analysis. t-distributed Stochastic Neighbor Embedding (t-SNE) analysis of spike-specific CD8 T-cells positive for at least one intracellular marker (IFN-γ, TNF-α, IL-4) from splenocytes of ifnar^{-/-} mice immunized with YF-S1/2 or YF-S0 (n=6 per group) after overnight stimulation with SARS-CoV-2 Spike peptide pool. Dots indicate IFN-γ expressing T-cells, TNF-α expressing T-cells, or IL-4 expressing CD8 T-cells. (H) Heatmap of IFN-γ expression density of spike-specific CD8 T-cells from YF-S1/2 and YF-S0 vaccinated mice. Scale bar represents IFN-γ expressing density (low expression to high expression) (see FIG. 15 for full analysis).

FIG. 7. Single shot vaccination in hamsters using the YF-S0 lead vaccine candidate. (A) Schematic presentation of experiment. Three groups of hamsters were vaccinated only once i.p. with sham (white; n=8) or YF-S0 at two different doses; 1×10³ PFU (low, circles; n=8) and 10⁴ PFU (high, triangles; n=8) of YF-S0 at 21 days prior to challenge. A fourth group was vaccinated with the high 10⁴ PFU dose of YF-S0 at 10 days prior to challenge (squares; n=8). (B-C) Humoral immune responses following single dose vaccination. Titers of nAb (B) and bAb (C) in sera collected from vaccinated hamsters immediately prior to challenge (minipools of sera of two to three animals analyzed for quantification of bAb). (D, E) Protection from SARS-CoV-2 infection. Protection from challenge with SARS-CoV-2 after vaccination with YF-S0 in comparison to sham vaccinated animals, as described for two-dose vaccination schedule (FIG. 3 and FIG. 12); log₁₀-fold change relative to sham vaccinated in viral RNA levels (D) and infectious virus loads (E) in the lung of vaccinated hamsters at day four p.i. as determined by RT-qPCR and virus titration, respectively. Dotted line indicating lower limit of quantification (LLOQ) or lower limit of detection (LLOD) as indicated. Data shown are medians±IQR. Statistical significance between groups was calculated by the non-parametric ANOVA, Kruskall-Wallis with uncorrected Dunn's test (ns=Non-Significant, P>0.05, * P<0.05, ** P<0.01, **** P<0.0001).

FIG. 8. Schematic representation of the YF17D-based vaccine candidates (YF-S). The SARS-CoV-2 Spike (S1/2, S0 or S1) antigen were inserted into the E/NS1 intergenic region as translational fusion within the YF17D polyprotein (dark grey) inserted in the ER (endoplasmic reticulum). To cope with topological constraints of the fold of both SARS-CoV-2 Spike antigens and the polyprotein of the YF17D vector, one extra transmembrane domain (derived from the West Nile virus E-protein; light grey) was added to the C-terminal cytoplasmic domain of the full-length S proteins (S1/2 and S0). Likewise, two transmembrane domains were fused to the ER-resident C-terminus of the S1 subunit in construct YF-S1. Scissors indicate proposed maturation cleavage sites, including the S1/2 furin-cleavage site deleted in YF-S0.

FIG. 9. Attenuation of YF-S vaccine candidates. (A) Weight evolution of suckling Balb/c mice (up to 21 days) after i.c. inoculation with 100 PFU of vaccine candidates (n=8) YF-S1/2 (3), YF-S0 (4), YF-S1 (5) in comparison to sham (n=10, grey, 1) or YF17D (n=9, black, 2). (B) Representative images of Balb/c mice at seven days after intracranial inoculation with sham, 10² PFU of either YF-S0 or YF17D. (C) Weight evolution of AG129 mice (up to 21 days) after intraperitoneal inoculation with a dose of 10², 10³ or 10⁴ PFU of YF-S0 (4; 5; 6), and 1, 10 or 10² PFU of YF17D (1; 2; 3; black and grey circles).

FIG. 10. Correlation of nAb titers as determined by plaque reduction neutralization test (PRNT) and by serum neutralization test (SNT). (A) Correlation analysis of nAb titers using SARS-CoV-2 (PRNT) and rVSV-AG-spike (SNT) for a panel of seven sera. SNT₅₀ and PRNT₅₀ values were plotted to determine the correlation between the neutralization assays with a Pearson regression coefficient of 0.77 (P=0.04). (B) NAbs in sera from four convalescent patients as determined by SNT. Data shown is median±IQR.

FIG. 11. Immunogenicity and protective efficacy in hamsters. (A) Virus RNA load in organs. Viral RNA in spleen, liver, kidney, heart and ileum of hamsters vaccinated with YF-S1/2, YF-S0 or sham, and challenged by infection with SARS-CoV-2. Viral RNA levels were determined by RT-qPCR, normalized against β-actin mRNA levels, and resulting fold-changes relative to the median of sham-vaccinated animals calculated using the 2^{(ΔΔCq)} method. (B-D) Anamnestic response. NAb titers (B) and bAbs titers (D) in hamsters immunized with YF-S1/2, YF-S0, YF-S1 in comparison to sham (white) or YF17D (yellow) four days after challenge with SARS-CoV-2. (C) Pair-wise comparison of nAb titers of sera collected at day 21 post-immunization (circles), and four days post-challenge (squares). For quantification of bAbs, minipools of sera of three animals each were analyzed. Statistical significance between groups was calculated by the non-parametric ANOVA, Kruskall-Wallis with uncorrected Dunn's test (A, B and D), or a non-parametric Wilcoxon matched-pairs rank test (C) (ns=Not-Significant,P>0.05, * P<0.05, ** P<0.01, *** P<0.001, **** P<0.0001).

FIG. 12. Immunogenicity and protective efficacy of vaccine candidate YF-S0 using a twice 5×10³ PFU dosing regimen. (A) Schematic representation of immunization and challenge schedule. Syrian hamsters were immunized twice i.p. at day 0 and 7 with 5×10³ PFU each of vaccine constructs YF-S0 (n=7), sham (white, n=3). At day 23 post-vaccination, animals were intranasally inoculated with 2×10⁵ TCID₅₀ of SARS-CoV-2 and followed up for four days. (B) Humoral immune responses. NAb titers 21 days post-vaccination. (C, D) Protection from SARS-CoV-2 infection. Viral loads in lungs of hamsters four days after intranasal infection were quantified by RT-qPCR (C) and virus titration (D) as in FIG. 3. Dotted line indicating lower limit of quantification (LLOQ) or lower limit of detection (LLOD) as indicated. Data shown are medians±IQR. Statistical significance between groups was calculated by the non-parametric two-tailed Mann-Whitney test (* P<0.05, ** P<0.01).

FIG. 13. Lung pathology by histology and micro-CT imaging. (A) Cumulative histopathology score for signs of lung damage (vasculitis, peri-bronchial inflammation, peri-vascular inflammation, bronchopneumonia, peri-vascular edema, apoptotic bodies in bronchus walls and intra-alveolar hemorrhage) in H&E stained lung sections (dotted line—maximum score in sham vaccinated group). (B) Representative micro-CT images of sham and YF-S0 vaccinated four days after SARS-CoV-2 infection. Arrows indicate examples of pulmonary infiltrates seen as consolidation of lung parenchyma (black and white).

FIG. 14. RNA expression levels after SARS-CoV-2 challenge. Individual expression profiles for 10 genes in lungs of vaccinated hamsters (n=12 per group) four days after SARS-CoV-2 infection (as in FIG. 4E) presented as log₁₀-fold change relative to uninfected controls (n=4). Levels of individual mRNAs were determined by RT-qPCR and normalized for β-actin mRNA. Changes are reported as values over the median of uninfected controls calculated using the 2^{(ΔΔCq)} method. Only for IFN-k, where all control animals had undetectable RNA levels, fold changes were calculated over the lowest detectable value. Data presented as median±IQR. LLOD— lower limit of detection (dotted line). Statistical significance compared to sham-vaccinated animals was calculated by a non-parametric ANOVA, Kruskall-Wallis with uncorrected Dunn's test (* P<0.05, ** P<0.01, *** P<0.001, **** P<0.0001).

FIG. 15. Profiling of CD8 and CD4 T-cells from YF-S1/2, YF-S0 and sham vaccinated mice by t-SNE analysis. Full representation of t-distributed Stochastic Neighbor Embedding (t-SNE) analysis of Spike-specific CD4 and CD8 T-cells positive for at least one intracellular marker (IFN-γ, TNF-α, IL-4, or IL17A) from splenocytes of YF-S1/2, YF-S0 and sham vaccinated ifnar^{-/-} mice (n=6 per group) after overnight stimulation with SARS-CoV-2 Spike peptide pool (IFN-γ expressing T-cells—TNF-α expressing T-cells—IL-4 expressing T-cells; yellow—IL17A expressing T-cells). t-SNE plots generated using FlowJo by first concatenating Spike-specific CD8 (upper panels) or CD4 T-cells (lower panels) from all animals.

FIG. 16. Sequential gating strategy for intracellular cytokine staining (ICS). First, live cells were selected by gating out Zombie Aqua (ZA) positive and low forward scatter (FSC) events. Then, doublets were eliminated in a FSC-H vs. FSC-A plot. T-cells (CD3 positive) were stratified into γδT-cells (γδTCR⁺), CD4 T-cells (γδTCR⁻/CD4⁺) and CD8 T-cells (γδTCR⁻/CD8⁺). Boundaries defining positive and negative populations for intracellular markers were set based on non-stimulated control samples.

FIG. 17. Humoral immune response elicited by YF in hamsters and mice. (A-B) Neutralizing antibodies (nAb) in hamsters (A) and ifnar^{-/-} mice (B) vaccinated with the different vaccine candidates (sera collected at day 21 post-vaccination in both experiments (two-dose vaccination schedule). (C) Quantitative assessment YF17D specific cell-mediated immune response by ELISpot. Spot counts for IFNγ-secreting cells per 10⁶ splenocytes after stimulation with a NS4B peptide. Dotted line indicating lower limit of quantification (LLOQ) as indicated. Data shown are medians±IQR. Statistical significance between groups was calculated by a non-parametric ANOVA, Kruskall-Wallis with uncorrected Dunn's test (ns=Not-Significant,P>0.05, * P<0.05, ** P<0.01, *** P<0.001).

FIG. 18. Lung pathology by histology. Cumulative histopathology score for signs of lung damage (vasculitis, peri-bronchial inflammation, peri-vascular inflammation, bronchopneumonia, peri-vascular edema, apoptotic bodies in bronchus walls) in H&E stained lung sections (dotted line—maximum score in sham-vaccinated group).

FIG. 19. Humoral and cellular immune response elicited by YF-S vaccine candidates in mice. (A) Schematic presentation of immunization and challenge schedule. Ifnar−/− mice were vaccinated once i.p. with 400 PFU YF-S0 (n=9), sham (white, n=6) or YF17D (grey, n=6). (B, C) SARS-CoV-2 specific antibody levels. Titers of nAbs (B) and bAbs (C) at day 21 post-vaccination; minipools of sera of two to three animals analyzed for quantification of bAb. (D) Quantitative assessment of SARS-CoV-2 specific CMI response by ELISpot. Spot counts for IFN-γ-secreting cells per 106 splenocytes after stimulation with SARS-CoV-2 Spike peptide pool. Data presented as median±IQR. Dotted line indicating lower limit of quantification (LLOQ) or lower limit of detection (LLOD). Statistical significance compared to sham-vaccinated animals was calculated by a one-way ANOVA, Kruskal-Wallis with uncorrected Dunn's test (* P<0.05, ** P<0.01).

FIG. 20. YF17D-specific humoral immune response elicited by YF-S in hamsters and mice. (A-B) Neutralizing antibodies (nAb) in hamsters (A) and ifnar−/− mice (B) vaccinated with the different vaccine candidates (sera collected at day 21 post-vaccination in both experiments (two-dose vaccination schedule)). (C) Quantitative assessment YF17D-specific cell-mediated immune response by ELISpot. Spot counts for IFNγ-secreting cells per 10⁶ splenocytes after stimulation with a YF17D NS4B peptide mixture. Dotted line indicating lower limit of quantification (LLOQ) as indicated. Data shown are medians±IQR. Statistical significance between groups was calculated by a one-way ANOVA, Kruskal-Wallis with uncorrected Dunn's test (ns=Not-Significant, P>0.05, * P<0.05, ** P<0.01, *** P<0.001).

FIG. 21. Longevity of the humoral immune response following single vaccination in hamster. A) Neutralizing antibody (nAbs) titers. B) Binding antibody titers (bAbs).

FIG. 22. Schematic overviews of constructs 1-7.

FIG. 23. Immunogenicity and protective efficacy in cynomolgus macaques. Twelve cynomolgus macaques (M. fascicularis) were immunized twice (at day 0 and day 7) subcutaneously with 10⁵ PFU of YF-S0 (n=6) or matched placebo (n=6). On day 21 after vaccination, all macaques were challenged with 1.5×10⁴ TCID50 SARS-CoV-2. a, NAbs on indicated days after first vaccination. Data are median±IQR. b, Virus RNA loads in throat swabs at indicated time points, quantified by RT-qPCR. Different symbols (squares, triangle and diamond) indicate values for individual macaques followed over time with virus RNA loads above the lower limit of quantification. Histological examination of the lungs (day 21 after challenge) revealed no evidence of any SARS-CoV-2-induced pathology in macaques vaccinated with either YF-S0 or placebo. Two-tailed uncorrected Kruskal-Wallis test was applied.

FIG. 24. Genetic stability of YF-S0 during passaging in BHK-21 cells. a, Schematic of YF-S0 passaging in BHK-21 cells. YF-S0 vaccine virus recovered from transfected BHK-21 cells (P0) was plaque-purified once (P1) (n=5 plaque isolates), amplified (P2) and serially passaged on BHK-21 cells (P3-P6). In parallel, each amplified plaque isolate (P2) (n=5) from the first plaque purification was subjected to a second round of plaque purification (P3*) (n=25 plaque isolates) and amplification (P4*). b, Schematic of tiled RT-PCR amplicons from three different primer pairs used for detection of the inserted SARS-CoV-2 S viral RNA sequence present in supernatants of different passages. All data are from a single representative experiment. c, RT-PCR fingerprinting performed on the virus supernatant collected from serial passage 3 (P3) and 6 (P6) of plaque-purified YF-S0. d, Immunoblot analysis of S expression by P3 and P6 of YF-S0. e, RT-PCR fingerprinting on amplified plaque isolates from the second round of plaque purification (P4*), 20 individual amplified plaque isolates are shown here (1-20). c, e, Control, YF-S0 cDNA (0.5 ng); ladder, 1-kb DNA ladder. Direct Sanger sequencing confirmed maintenance of full-length S inserts for 25 out of 25 plaques (100%). After two rounds of plaque purification and amplification, only in three isolates a single point mutation was found (two silent mutations and one missense mutation resulting in a S47P amino acid change in the N terminus of S1); at a low <10⁻⁴ mutation frequency (that is, 3 nt changes observed in a total of 25×4,196 nt=104,900 nt sequenced; of which 25×3,780 nt=94,500 nt were of S transgene sequence). This mutation rate is similar to that of parental YF17D under current vaccine manufacturing conditions 14,78.

FIG. 25. Attenuation of YF-S vaccine candidates. a, Survival curve of wild-type (WT) and STAT2-knockout (STAT2^{-/-}) hamsters inoculated intraperitoneally with 10⁴ PFU of YF17D or YF-S0. Wild-type hamsters inoculated with YF17D (n=6) and YF-S0 (n=6); STAT2^{-/-} hamsters inoculated with YF17D (n=14) and YF-S0 (n=13). The number of surviving hamsters at study end point is indicated. b, c, Vaccine virus RNA (viraemia) in the serum (b) and weight evolution (c) of wild-type hamsters after intraperitoneal inoculation with 10⁴ PFU YF17D (n=6) or YF-S0 (n=6). The number of hamsters that showed viraemia on each day after inoculation is indicated below (b). d, Weight evolution of Ifnar^{-/-} mice after intraperitoneal inoculation with 400 PFU each at day 0 and 7 of YF-S0, YF17D and sham. Mice were inoculated with YF17D (n=5), YF-S0 (n=5) or sham (n=5). Data in a are from two independent experiments, data in other panels are from a single experiment.

FIG. 26. Immunogenicity and protective efficacy in hamsters after single dose vaccination a, b, Hamsters (n=6 per group from a single experiment) were vaccinated with a single dose of YF-S0 (10⁴ PFU intraperitoneally) and sera were collected at 3, 10 and 12 weeks after vaccination. NAbs (a) and binding antibodies (b) at the indicated weeks post vaccination. Data are median±IQR. Two-tailed uncorrected Kruskal-Wallis test was applied.

FIG. 27. YF17D specific immune responses I macaques a, b, NAb titres after vaccination in macaques with YF-S0 (a) or placebo (b) (6 macaques per group from a single experiment); sera collected at indicated times after vaccination (two-dose vaccination schedule; FIG. 7). c, Ifnar^{-/-} mice vaccinated according to a single-dose vaccination schedule (YF-S0 (n=8), sham (n=5) and YF17D (n=5) from 2 independent experiments). Spot counts for IFNγ-secreting cells per 10⁶ splenocytes after stimulation with a YF17D NS4B peptide mixture. Data are median±IQR. Two-tailed uncorrected Kruskal-Wallis test was applied.

FIG. 28. Protection from lethal YF17D. a, Ifnar^{-/-} mice were vaccinated with either a single 400 PFU intraperitoneal (i.p.) dose of YF17D (black) (n=7) or YF-S0 (n=10), or sham (grey, n=9). After 21 days, mice were challenged by intracranial (i.c.) inoculation with a uniformly lethal dose of 3×10³ PFU of YF17D and monitored for weight evolution (b) and survival (c). The number of surviving mice at study end point (day 15) is indicated. Data are from two independent experiments.

FIG. 29 Sequences of constructs of Example 2.

### DETAILED DESCRIPTION OF THE INVENTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation is meant to apply throughout this specification, i.e., also in the context of other aspects or embodiments of the invention, unless otherwise defined.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

Present inventors have found that a polynucleotide comprising a nucleotide sequence of a live, infectious, attenuated Flavivirus, wherein a nucleotide sequence encoding at least a part of a coronavirus Spike protein, preferably encoding the S1 and S2 unit (such as in their native cleavable version or in a non-cleavable version), is inserted, so as to allow expression of a chimeric virus from said polynucleotide, can be used in the preparation of a vaccine against a coronavirus, such as the SARS-CoV2 virus. A surprisingly high safety profile, immunogenicity and efficacy could be obtained in vivo for such vaccines encoding both the S1 and S2 unit.

Furthermore, present inventors found that mutating the S1/2 cleavage site to prevent proteolytic processing of the S protein in the S1 and S2 subunits, allows to keep the spike protein in a stabilized non-cleavable form and that this contributes to the induction of a robust immune response in vivo and the protection against stringent coronavirus challenge, such as a SARS-CoV-2 challenge. For example, present inventors have used live-attenuated yellow fever 17D (YF17D) vaccine as a vector to express the non-cleavable prefusion form of the SARS-CoV-2 spike antigen (comprising both the S1 and S2 subunits). Such vaccine has an excellent safety profile. This ensures that the vaccine is also suitable for those persons who are most vulnerable to COVID-19, such as all people aged nine months or older who live in areas at risk, including elderly individuals and persons with underlying medical conditions). The vaccine also has a superior immunogenicity, and a superior efficacy, for example compared to a vaccine comprising the cleavable form of the SARS-CoV-2 spike antigen. Moreover, such vaccine efficiently prevents systemic viral dissemination, prevents increase in cytokines linked to disease exacerbation in COVID-19, and/or offers a considerable longevity of immunity induced by a single-dose vaccination. In addition, such vaccine has a markedly reduced neurovirulence, such as when compared to a vaccine comprising the cleavable form of the SARS-CoV-2 spike ntigen. Therefore, such vaccine might be ideally suited for population-wide immunization programs.

More particularly, present inventors have shown that such vaccine expressing the non-cleavable prefusion form of the SARS-CoV-2 spike antigen induces high levels of ARS-CoV-2 neutralizing antibodies in vivo, as was shown in hamsters (Mesocricetus auratus), mice (Mus musculus) and cynomolgus macaques (Macaca fascicularis), and—concomitantly—protective immunity against yellow fever virus. Moreover, using such vaccine, humoral immunity is complemented by a cellular immune response with favourable T helper 1 polarization, as profiled by present inventors in mice. In a hamster model and in macaques, such vaccine has been shown to prevent infection with SARS-CoV-2. Moreover, a single dose conferred protection from lung disease in most of the vaccinated hamsters within as little as 10 days.

A first aspect provides a polynucleotide comprising a sequence of (i.e. a nucleotide sequence encoding) a live, infectious, attenuated Flavivirus wherein a nucleotide sequence encoding at least a part of a coronavirus Spike protein is inserted (i.e. is located), so as to allow expression of a chimeric virus from said polynucleotide. Accordingly, the polynucleotide as taught herein therefore encodes a chimeric virus and comprises a sequence of a live, infectious, attenuated Flavivirus and a nucleotide encoding at least a part of a coronavirus Spike protein.

A further aspect provides a polynucleotide comprising a sequence of (i.e. a nucleotide sequence encoding) a live, infectious, attenuated Flavivirus wherein a nucleotide sequence encoding an antigen of at least 1000 amino acids, at least 1100 amino acids, at least 1200 amino acids, or at least 1250 amino acids, is inserted (i.e. is located), so as to allow expression of a chimeric virus from said polynucleotide.

The term “inserted” or “insertion” or “inserting” (i.e. located) as used herein refers to the inclusion (location) of the nucleotide sequence encoding at least a part of a coronavirus Spike protein within a nucleotide sequence encoding a component of the Flavivirus, in between two nucleotide sequences each encoding different components of the flavivirus or prior to (upstream) of the sequence encoding the flavivirus. The term “inserted in between” (i.e. located in between) as used herein refers to the inclusion (location) of the nucleotide sequence encoding at least part of a coronavirus Spike protein in between two other encoding nucleotide sequences, such as sequences encoding different components of the flavivirus (e.g. C, prM, E, NS1, NS, NS2A, NS2B, NS3, NS4A, NS4B, or NS5), preferably so that the nucleotide sequence encoding at least part of a coronavirus Spike protein comprises 5′ and 3′ a nucleotide sequence encoding a component of the flavivirus. For example, the term “inserted in between” (i.e. located in between) may be used to refer to the insertion (location) of the nucleotide encoding at least a part of a coronavirus Spike protein in between the E protein and the NS1 protein of the flavivirus (i.e. in the E/NS1 boundary of the flavivirus). In particular embodiments, the term “inserted” does not encompass a substitution of one or more nucleotide sequences by other nucleotide sequence(s).

The term “nucleic acid” or “polynucleotide” as used throughout this specification typically refers to a polymer (preferably a linear polymer) of any length composed essentially of nucleoside units. A nucleoside unit commonly includes a heterocyclic base and a sugar group. Heterocyclic bases may include inter alia purine and pyrimidine bases such as adenine (A), guanine (G), cytosine (C), thymine (T) and uracil (U) which are widespread in naturally-occurring nucleic acids, other naturally-occurring bases (e.g., xanthine, inosine, hypoxanthine) as well as chemically or biochemically modified (e.g., methylated), non-natural or derivatised bases. Nucleic acid molecules comprising at least one ribonucleoside unit may be typically referred to as ribonucleic acids or RNA. Such ribonucleoside unit(s) comprise a 2′-OH moiety, wherein —H may be substituted as known in the art for ribonucleosides (e.g., by a methyl, ethyl, alkyl, or alkyloxyalkyl). Preferably, ribonucleic acids or RNA may be composed primarily of ribonucleoside units, for example, >80%, >85%, >90%, >95%, >96%, >97%, >98%, >99% or even 100% (by number) of nucleoside units constituting the nucleic acid molecule may be ribonucleoside units. Nucleic acid molecules comprising at least one deoxyribonucleoside unit may be typically referred to as deoxyribonucleic acids or DNA. Such deoxyribonucleoside unit(s) comprise 2′-H. Preferably, deoxyribonucleic acids or DNA may be composed primarily of deoxyribonucleoside units, for example, ≥80%, ≥85%, ≥90%, ≥95%, ≥96%, ≥97%, ≥98%, ≥99% or even 100% (by number) of nucleoside units constituting the nucleic acid molecule may be deoxyribonucleoside units. Nucleoside units may be linked to one another by any one of numerous known inter-nucleoside linkages, including inter alia phosphodiester linkages common in naturally-occurring nucleic acids, and further modified phosphate- or phosphonate-based linkages.

The term “nucleic acid” further preferably encompasses DNA, RNA and DNA/RNA hybrid molecules, specifically including hnRNA, pre-mRNA, mRNA, cDNA, genomic DNA, amplification products, oligonucleotides, and synthetic (e.g., chemically synthesised) DNA, RNA or DNA/RNA hybrids. RNA is inclusive of RNAi (inhibitory RNA), dsRNA (double stranded RNA), siRNA (small interfering RNA), mRNA (messenger RNA), miRNA (micro-RNA), tRNA (transfer RNA, whether charged or discharged with a corresponding acylated amino acid), and cRNA (complementary RNA). A nucleic acid can be naturally occurring, e.g., present in or isolated from nature, e.g., produced natively or endogenously by a cell or a tissue and optionally isolated therefrom. A nucleic acid can be recombinant, i.e., produced by recombinant DNA technology, and/or can be, partly or entirely, chemically or biochemically synthesised. Without limitation, a nucleic acid can be produced recombinantly by a suitable host or host cell expression system and optionally isolated therefrom (e.g., a suitable bacterial, yeast, fungal, plant or animal host or host cell expression system), or produced recombinantly by cell-free transcription, or non-biological nucleic acid synthesis. A nucleic acid can be double-stranded, partly double stranded, or single-stranded. Where single-stranded, the nucleic acid can be the sense strand or the antisense strand. In addition, nucleic acid can be circular or linear.

Flaviviruses have a positive single-strand RNA genome of approximately 11,000 nucleotides in length. The genome contains a 5′ untranslated region (UTR), a long open-reading frame (ORF), and a 3′ UTR. The ORF encodes three structural (capsid [C] (or core), precursor membrane [prM], and envelope [E]) and seven nonstructural (NS1, NS2A, NS2B, NS3, NS4A, NS4B, and NS5) proteins. Along with genomic RNA, the structural proteins form viral particles. The nonstructural proteins participate in viral polyprotein processing, replication, virion assembly, and evasion of host immune response. The signal peptide at the C terminus of the C protein (C-signal peptide; also called C-anchor domain) regulates Flavivirus packaging through coordination of sequential cleavages at the N terminus (by viral NS2B/NS3 protease in the cytoplasm) and C terminus (by host signalase in the endoplasmic reticulum [ER] lumen) of the signal peptide sequence.

The positive-sense single-stranded genome is translated into a single polyprotein that is co- and post translationally cleaved by viral and host proteins into three structural [Capsid (C), premembrane (prM), envelope (E)], and seven non-structural (NS1, NS2A, NS2B, NS3, NS4A, NS4B, NS5) proteins. The structural proteins are responsible for forming the (spherical) structure of the virion, initiating virion adhesion, internalization and viral RNA release into cells, thereby initiating the virus life cycle. The non-structural proteins on the other hand are responsible for viral replication, modulation and evasion of immune responses in infected cells, and the transmission of viruses to mosquitoes. The intra- and inter-molecular interactions between the structural and non-structural proteins play key roles in the virus infection and pathogenesis.

The E protein comprises at its C terminal end two transmembrane sequences, indicated as TM1 and TM2.

NS1 is translocated into the lumen of the ER via a signal sequence corresponding to the final 24 amino acids of E and is released from E at its amino terminus via cleavage by the ER resident host signal peptidase (Nowak et al. (1989) Virology 169, 365-376). The NS1 comprises at its C terminal a 8-9 amino acids signal sequence which contains a recognition site for a protease (Muller & Young (2013) Antiviral Res. 98, 192-208).

A sequence of a live, infectious, attenuated Flavivirus may refer to a nucleotide sequence encoding all components of a Flavivirus required for the formation of a live, infectious, attenuated Flavivirus, such as a live, infectious, attenuated YF17D virus. The full length YF17D sequence is for example as annotated under NCBI Genbank accession number X03700.1.

Infectious viruses are typically capable of infecting a host cell. “Attenuation” in the context of the present invention relates to the change in the virulence of a pathogen by which the harmful nature of disease-causing organisms is weakened (or attenuated); attenuated pathogens can be used as life vaccines. Attenuated vaccines can be derived in several ways from living organisms that have been weakened, such as from cultivation under sub-optimal conditions (also called attenuation), or from genetic modification, which has the effect of reducing their ability to cause disease.

In particular embodiments, the sequence of a live, infectious, attenuated Flavivirus comprises a sequence encoding a capsid (C) protein or a part thereof, a premembrane (prM) protein, an envelope (E), a NS1 non-structural protein, a NS2A non-structural protein, a NS2B non-structural protein, a NS3 non-structural protein, an NS4A non-structural protein, a NS4B non-structural protein, a NS5 non-structural protein of a Flavivirus. The present invention is exemplified with chimeric constructs of a YFV 17D backbone, S antigen of Covid-19 and TM domains of West Nile virus.

The similarity in sequences inbetween flavivirus and inbetween S antigens of coronaviruses allow, allow the construction of chimeric construct with backbones other than YFV, TM domains other than West Nile Virus, and S antigens other that Covid-19. The present invention allow the generation of DNA vaccines against coronaviruses such as severe acute respiratory syndrome coronavirus (SARS-CoV) (e.g. SARS-CoV2), HCoV NL63, HKU1 and MERS-CoV.

In particular embodiments, the coronavirus is COVID-19 (or SARS-CoV2).

The person skilled in the art that the Spike protein may be the Spike protein of any variant of the SARS-CoV2 virus.

In particular embodiments, the Spike protein is the Spike protein from the SARS-CoV-2 strain BetaCov/Belgium/GHB-03021/2020 sequence which is available from GISAID (EPI ISL 407976|2020-02-03) (https://www.gisaid.org), the Spike protein from the SARS-CoV-2 isolate Wuhan-Hu-1 as as annotated under NCBI Genbank accession number MN908947.3, the Spike protein from the UK variant of the SARS-CoV2 virus (e.g. VOC 202012/01, B.1.1.7), the Spike protein from the Brazilian-Japanese variant of the SARS-CoV2 virus (e.g. B.1.1.248), the Spike protein of the South African variant of the SARS-CoV2 virus (e.g. VOC 501Y.V2, B. 1.351), the Spike protein of the Californian variant of the SARS-CoV2 virus or the Spike protein of the New York variant of the SARS-CoV2 variant.

An exemplary annotated nucleotide sequence and amino acid sequence of COVID-19 (or SARS-CoV-2) Spike (S) is depicted below.

Nucleotide sequence:

### Signal Peptide (15 Aa), SUBUNIT-1, Cleavage S1/S2, Subunit-2/S2′ (KR)/Fusion Peptide

As described elsewhere in the present specification, in the vaccine constructs of the present invention the first 13aa (39 nucleotides in lowercase) of the SP may be deleted, preferably in the vaccine constructs of the present invention only the first 13aa (39 nucleotides in lowercase) of the SP are deleted

SEQ ID NO: 1
atgtttgttttcttggtcctccttccactggtatcctcaCAATGTGTGAA
TCTTACCACCCGAACCCAGCTTCCTCCCGCCTACACGAATTCATTCACGC
GGGGTGTTTATTACCCGGATAAAGTTTTCCGGTCCAGTGTCCTGCATTCA
ACCCAAGACCTCTTTCTGCCATTTTTTTCTAACGTGACGTGGTTCCATGC
TATCCATGTAAGCGGAACCAACGGAACCAAACGGTTCGATAATCCGGTTC
TCCCATTCAACGATGGGGTTTACTTCGCATCTACAGAAAAATCTAACATA
ATTAGAGGATGGATTTTTGGGACTACGCTTGACAGTAAGACCCAATCACT
CTTGATCGTGAACAATGCAACCAATGTAGTAATTAAGGTTTGCGAGTTTC
AATTTTGTAATGATCCATTTTTGGGGGTTTATTACCACAAAAACAATAAA
TCCTGGATGGAATCCGAATTCAGAGTGTATAGCAGCGCTAACAATTGCAC
ATTTGAGTACGTGTCACAACCTTTTCTTATGGATCTTGAGGGCAAGCAAG
GGAACTTCAAAAATTTGAGGGAGTTCGTTTTCAAGAACATCGACGGATAC
TTTAAGATCTATTCTAAACACACCCCCATTAACTTGGTGCGAGATTTGCC
TCAAGGCTTCTCTGCACTTGAACCGTTGGTGGATCTTCCCATTGGCATTA
ATATTACTCGGTTCCAGACTTTGTTGGCACTGCATCGCTCCTATCTCACG
CCCGGAGACAGTTCATCTGGATGGACTGCGGGGGCTGCCGCGTATTACGT
GGGATACCTGCAGCCGCGCACATTTCTTCTTAAATACAACGAGAACGGGA
CCATCACAGATGCAGTGGATTGCGCTCTTGACCCCCTCTCCGAAACAAAA
TGTACGCTCAAGTCTTTCACTGTAGAGAAAGGGATTTATCAGACATCCAA
TTTCCGAGTCCAGCCAACAGAGAGTATAGTGCGGTTCCCTAACATCACAA
ATCTTTGTCCGTTCGGGGAAGTTTTCAACGCTACACGCTTCGCAAGTGTA
TACGCTTGGAATAGAAAGAGGATCTCTAATTGTGTGGCAGATTACTCTGT
GCTCTACAATTCCGCATCTTTCTCAACCTTCAAGTGTTACGGAGTTTCAC
CTACGAAGCTGAACGACCTTTGCTTTACTAATGTATATGCAGATAGTTTT
GTCATCAGGGGCGATGAAGTTCGACAGATAGCGCCCGGCCAGACAGGAAA
GATCGCGGACTACAATTATAAACTCCCTGATGATTTCACCGGGTGCGTGA
TCGCGTGGAACTCTAATAACCTCGACTCCAAAGTAGGCGGTAACTACAAT
TACTTGTATCGATTGTTTAGAAAATCAAACCTTAAACCGTTCGAGCGGGA
TATCTCTACCGAGATATACCAAGCAGGCTCTACACCGTGCAATGGAGTCG
AAGGTTTTAACTGCTACTTCCCCTTGCAATCATACGGGTTTCAGCCTACC
AACGGTGTAGGATACCAGCCTTACAGGGTTGTGGTACTTTCATTTGAGCT
CCTGCACGCTCCCGCAACTGTCTGTGGGCCCAAAAAGAGCACTAACCTTG
TTAAAAATAAATGCGTCAACTTTAACTTCAATGGCCTCACTGGCACCGGC
GTGCTCACTGAAAGCAATAAGAAATTCCTTCCTTTTCAGCAGTTTGGGCG
AGACATAGCGGATACCACGGACGCAGTACGGGATCCTCAAACCCTTGAAA
TCCTTGACATAACGCCTTGCTCTTTTGGGGGAGTAAGCGTAATCACGCCT
GGAACCAACACCTCCAATCAGGTTGCTGTGCTGTACCAGGATGTAAACTG
CACCGAGGTACCGGTAGCCATTCACGCGGATCAGCTGACTCCCACATGGC
GAGTGTATTCTACAGGTAGTAATGTGTTTCAGACCCGAGCAGGGTGTTTG
ATAGGGGCGGAGCACGTCAACAACTCATACGAGTGCGATATACCCATTGG
GGCTGGTATATGTGCATCCTACCAGACGCAGACGAACTCTCCT [symbol]
tctgttgcatctcaatcaattattgcatacactatgt
cactgggggctgagaattcagtagcctactctaacaacagcatcgcgatt
cccactaacttcacaattagtgtgactaccgagatcctgccagtatccat
gactaaaactagcgtagattgtactatgtacatctgcggcgattcaactg
agtgttcaaacctcctcttgcaatacgggtcattttgtacccaattgaat
cgagctctgaccggcatcgcggtcgaacaggacaaaaatactcaagaggt
atttgcccaggtgaagcagatttacaaaacaccccctatcaaggatttcg
ggggcttcaacttcagccagatactgccagacccctcaaaaccgagcAAG
CGCtccttcatagaagatcttcttttcaacaaagttaccctcgcggatgc
tggtttcattaaacagtatggggactgtctcggcgacattgctgctagag
acctcatctgcgcgcaaaagttcaatggacttacggtcctgccccctctc
ctcactgatgaaatgattgctcaatatacgtccgcgttgttggcgggaac
tataaccagtgggtggacgttcggcgctggcgccgcgcttcaaatcccat
ttgcgatgcaaatggcgtatcgcttcaacggcatcggagtaactcaaaac
gttctgtacgaaaatcaaaaactcattgcgaaccagtttaattcagcgat
cggtaaaatccaggacagcctgagctccacagcgagtgcactcgggaagc
tccaggatgtggtaaatcagaacgctcaagcgttgaacacactcgtcaag
cagctgtcaagtaactttggcgcgatttcatctgtattgaatgacattct
ctctcgccttgataaggtggaagccgaagtccagattgatcgcctgatta
ctgggcggcttcagtccctccaaacatacgtcactcagcaacttattaga
gccgccgaaattagggcaagtgcgaatctggccgcgacaaaaatgtctga
atgtgtgctggggcagagcaagagagtcgatttttgcggtaaggggtatc
accttatgtcttttcctcagtctgcccctcacggagtagtgtttctccac
gttacgtatgtcccagcccaagagaaaaactttaccactgcgccggctat
ttgtcatgacggtaaagcacactttccacgcgaaggtgtgttcgtctcca
acggcacccactggtttgtaacgcagaggaacttctacgagcctcagata
attaccacggacaacacgttcgtctcaggtaactgcgacgtcgtaattgg
tattgtaaataacaccgtgtacgacccgctccagccggagctggactcct
tcaaagaggagcttgacaagtattttaagaatcacacttcaccggatgta
gacctgggggatatttccggcataaacgcttccgtggttaacatacagaa
agagatagatcgactgaacgaggtagcgaaaaacttgaatgagtctttga
tagacctgcaagaattgggaaaatatgaacaatatattaagtggccctgg
tatatttggcttggtttcatagccggtttgattgccatcgtcatggtaac
tataatgctttgttgcatgacaagttgctgctcttgcctcaaagggtgct
gctcctgtggaagttgttgcaagttcgatgaggatgattctgagccagtg
cttaagggtgtcaaactgcattatacg
Amino acid sequence
SEQ ID NO: 2
mfvflvllplvssQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFEMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSP [symbol] svasqsiiavtmslg
aensvaysnnsiaiptnftisvtteilpvsmtktsvdctmyicgdstecs
nlllqygsfctqlnraltgiaveqdkntqevfaqvkqiyktppikdfggf
nfsqilpdpskpsKRsfiedllfnkvtladagfikqygdclgdiaardli
caqkfngltvlpplltdemiaqytsallagtitsgwtfgagaalqipfam
qmayrfngigvtqnvlyenqklianqfnsaigkiqdslsstasalgklqd
vvnqnaqalntlvkqlssnfgaissvlndilsrldkveaevqidrlitgr
lqslqtyvtqqliraaeirasanlaatkmsecvlgqskrvdfcgkgyhlm
sjpqsaphgvvflhvtyvpaqeknfttapaichdgkahjpregvjvsngt
hwjvtqrnjyepqiittdntjvsgncdvvigivnntvydplqpeldsfke
eldkyfknhtspdvdlgdisginasvvniqkeidrlnevaknlneslidl
qelgkyeqyikwpwyiwlgftagliaivmvtimlccmtsccsclkgccsc
gscckfdeddsepvlkgvklhyt

For example, the mutations (amino acid) in the SARS-CoV2 variants United Kingdom (VOC 202012/01, B.1.1.7), South-Africa (VOC 501Y.V2, B. 1.351) and Brazilian-Japanese (B.1.1.248) in comparison with the Spike sequence as defined by SEQ ID NO: 2 are typically the following:
UK variant compared to SEQ ID NO:2: deletion of amino acids 69-70 and 144, and amino acid substitutions: N501Y, A570D, D614G, P681H, T716I, S982A, and D1118H;
South Africa (SA) variant compared to SEQ ID NO:2: deletion of amino acids 242-244, and amino acid substitutions: L18F, D80A, D215G, R246I, K417N, E484K, N501Y, D614G, and A701V; or
Brazilian-Japanese (BR) variant compared to SEQ ID NO:2: amino acid substitutions: L18F, T20N, P26S, D138Y, R190S, K417T, E484K, N501Y, D614G, H655Y, T1027I, V1176F; wherein the number indicates the respective amino acid of SEQ ID NO: 2 (i.e. including the signal peptide).

In particular embodiments, the at least part of the coronavirus Spike protein is at least the S2 subunit of a coronavirus Spike protein. In more particular embodiments, the at least part of the coronavirus Spike protein is at least the S2 subunit of the COVID-19 (or SARS-CoV-2) Spike protein, preferably at least the S2 subunit of the COVID-19 (or SARS-CoV-2) Spike protein comprising, consisting essentially of, or consisting of SEQ ID NO: 17, or the corresponding part in SEQ ID NO: 98, SEQ ID NO: 100 or SEQ ID NO: 102.

In other words, in particular embodiments, the polynucleotide as taught herein comprises a nucleotide sequence encoding at least the S2 subunit of a coronavirus Spike protein. In more particular embodiments, the polynucleotide as taught herein comprises a nucleotide sequence encoding at least the S2 subunit of the COVID-19 (or SARS-CoV-2) Spike protein. In even more particular embodiments, the polynucleotide as taught herein comprises a nucleotide sequence as defined by SEQ ID NO: 17, or the corresponding part in SEQ ID NO: 98, SEQ ID NO: 100 or SEQ ID NO: 102.

The at least part of the coronavirus Spike protein is preferably capable of forming a protein trimer. Furthermore, present inventors demonstrated that the presence of both the S1 and S2 unit is preferred to elicit an adequate humoral immune response.

Accordingly, in particular embodiments, the at least part of the coronavirus Spike protein comprises, consists essentially of or consists of the S1 and the S2 subunit of a coronavirus Spike protein. In more particular embodiments, the at least part of the coronavirus Spike protein comprises, consists essentially of or consists of the S1 and the S2 subunit of the COVID-19 (or SARS-CoV-2) Spike protein.

In other words, in particular embodiments, the polynucleotide as taught herein comprises a nucleotide sequence encoding the S1 and the S2 subunit of a coronavirus Spike protein. In more particular embodiments, the polynucleotide as taught herein comprises a nucleotide sequence encoding the S1 and the S2 subunit of the COVID-19 (or SARS-CoV-2) Spike protein. In even more particular embodiments, the polynucleotide as taught herein comprises a nucleotide sequence as defined by SEQ ID NO: 17 and a nucleotide as defined by SEQ ID NO: 18, or the corresponding parts in SEQ ID NO: 98, SEQ ID NO: 100 or SEQ ID NO: 102.

In particular embodiments, the nucleotide sequence consecutively encodes the S1 and S2 subunit of the coronavirus Spike protein. The skilled person will understand that this means that the sequence encoding the S1 subunit is located 5′ of the sequence encoding the S2 subunit. The nucleotide sequence consecutively encoding the S1 and S2 subunit will typically comprise a S1/S2 cleavage site formed by the 3′ end of the S1 subunit and the 5′ end of the S2 subunit of the coronavirus Spike protein. Accordingly, in particular embodiments, the polynucleotide as taught herein comprises a nucleotide sequence as defined by SEQ ID NO: 19. As described elsewhere in the present specification, this S1/S2 cleavage site may be mutated to prevent proteolytic processing of the S protein in the S1 and S2 subunits. Accordingly, in particular embodiments, the polynucleotide as taught herein comprises a nucleotide sequence as defined by SEQ ID NO: 97. In particular embodiments, the nucleotide sequence encoding at least part of the coronavirus Spike protein comprises the full length sequence of the precursor form (i.e. including the full length signal peptide or a part thereof) of the coronavirus spike protein.

In particular embodiments, the nucleotide sequence encoding at least part of the coronavirus Spike protein does not comprise the nucleotide sequence encoding the signal peptide or part of the signal peptide of the coronavirus Spike protein. The signal peptide of a coronavirus Spike protein typically comprises, consists essentially of or consists of 45 nucleotides (encoding 15 amino acids). Accordingly, the nucleotide sequence encoding the signal peptide or part of the signal peptide of a coronavirus Spike protein may comprise from 1 to 45 nucleotides, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides, preferably 6 nucleotides.

In particular embodiments, the nucleotide sequence encoding at least part of a coronavirus Spike protein comprises the last (or most 3′) six nucleotides of the nucleotide sequence encoding the signal peptide of the Spike protein, such as comprising the sequence 5′-CAATGT-3′.

In particular embodiments, the nucleotide sequence encoding the at least part of a coronavirus Spike protein does not comprise the first 39 nucleotides of the nucleotide sequence encoding the signal peptide of the Spike protein.

In particular embodiments, the nucleotide sequence encoding at least part of a coronavirus Spike protein does not comprise a sequence as defined by SEQ ID NO: 20 5′ (upstream) of the nucleotide sequence encoding the at least part of the coronavirus Spike protein.

A coronavirus infects a target cell by either cytoplasmic or endosomal membrane fusion. The final step of viral entry into the host cell involves the release of RNA into the cytoplasm for replication. Therefore, the fusion capacity of the coronavirus Spike protein is an important indicator of infectivity of the corresponding virus. The S1 and S2 subunit of the coronavirus Spike protein are typically separated by a S1/S2 cleavage site. The coronavirus Spike protein needs to be primed through cleavage at S1/S2 site and S2′ site in order to mediate the membrane fusion. For example, in the SARS-CoV-2 Spike protein, the S1 and S2 subunit are separated by a cleavage site comprising, consisting essentially of or consisting of the nucleotide sequence CGCCGCGCTCGG (SEQ ID NO: 21), which is a unique furin-like cleavage site (FCS).

Present inventors found that the non-cleavable form of the Spike protein is advantageous for the preparation of a vaccine with an excellent safety profile, immunogenicity and efficacy.

In particular embodiments, such as wherein the polynucleotide as taught herein comprises a nucleotide sequence encoding the S1 and S2 subunit of the coronavirus Spike protein, the nucleotide sequence encoding the S1/S2 cleavage site is mutated, thereby preventing proteolytic processing of S protein in the S1 and S2 subunits.

In particular embodiments, such as wherein the polynucleotide as taught herein comprises a nucleotide sequence encoding the S1 and S2 subunit of the coronavirus Spike protein, the nucleotide sequence encoding the S1/2 cleavage site is mutated from the nucleotide sequence CGCCGCGCTCGG (SEQ ID NO: 21) to the nucleotide sequence GCCGCCGCTGCG (SEQ ID NO: 22).

In particular embodiments, such as wherein the polynucleotide as taught herein comprises a nucleotide sequence encoding the S1 and S2 subunit of the coronavirus Spike protein, the S1/2 cleavage site is mutated from the amino acid sequence RRAR (SEQ ID NO: 23) to the amino acid sequence AAAA (SEQ ID NO: 24). The S1/S2 cleavage site may also be mutated to SGAG (SEQ ID NO: 91), such as described in McCallum et al., Structure-guided covalent stabilization of coronavirus spike glycoprotein trimers in the closed formation, Nature structural and molecular biology, 2020, or to GSAS (SEQ ID NO: 92) or to a single R, such as described in Xiong et al., A thermostable, closed SARS-CoV-2 spike protein trimer, Natural Structural & Molecular Biology, 2020.

In particular embodiments, such as wherein the polynucleotide as taught herein comprises a nucleotide sequence encoding at least the S2 subunit of the coronavirus Spike protein, the nucleotide sequence encoding the S2′ cleavage site in the S2 subunit of the coronavirus Spike protein is mutated, thereby preventing proteolytic processing of the S2 unit.

In particular embodiments, such as wherein the polynucleotide as taught herein comprises a nucleotide sequence encoding at least the S2 subunit of the coronavirus Spike protein, the nucleotide sequence encoding the S2′ cleavage site in the S2 subunit of the coronavirus Spike protein is mutated from 5′-AAGCGC-3′ to 5′-GCGAAC-3′.

In particular embodiments, such as wherein the polynucleotide as taught herein comprises a nucleotide sequence encoding at least the S2 subunit of the coronavirus Spike protein, the nucleotide sequence encoding the S2′ cleavage site in the S2 subunit of the coronavirus Spike protein is not mutated. Accordingly, in particular embodiments, the nucleotide sequence encoding the S2′ cleavage site in the S2 subunit of the coronavirus Spike protein comprises a sequence 5′-AAGCGC-3′.

In particular embodiments, the nucleotide sequence encoding at least part of a coronavirus Spike protein encodes the spike protein S2 subunit of the coronavirus Spike protein and does not encode the spike protein S1 subunit of the coronavirus Spike protein. In more particular embodiments, the nucleotide sequence encoding at least part of a coronavirus Spike protein encodes the spike protein S2 subunit of the SARS-CoV2 virus and does not encode the spike protein S1 subunit of the SARS-CoV2 virus. In particular embodiments, the polynucleotide sequence as taught herein does not comprise a nucleotide sequence as defined by SEQ ID NO: 18, or the corresponding part in SEQ ID NO: 98, SEQ ID NO: 100 or SEQ ID NO: 102.

In particular embodiments, the nucleotide sequence encoding at least part of a coronavirus Spike protein encodes the spike protein S1 subunit of the coronavirus Spike protein and does not encode the spike protein S2 subunit of the coronavirus Spike protein. In more particular embodiments, the nucleotide sequence encoding at least part of a coronavirus Spike protein encodes the spike protein S1 subunit of the SARS-CoV2 virus and does not encode the spike protein S2 subunit of the SARS-CoV2 virus. In particular embodiments, the polynucleotide sequence as taught herein does not comprise a nucleotide sequence as defined by SEQ ID NO: 17, or the corresponding part in SEQ ID NO: 98, SEQ ID NO: 100 or SEQ ID NO: 102.

The present invention is illustrated with a yellow fever virus, more particularly the yellow fever 17 D (YF17D) virus, but can be equally performed using other flavivirus based constructs such as but not limited to, Japanese Encephalitis, Dengue, Murray Valley Encephalitis (MVE), St. Louis Encephalitis (SLE), West Nile (WN), Tick-borne Encephalitis (TBE), Russian Spring-Summer Encephalitis (RSSE), Kunjin virus, Powassan virus, Kyasanur Forest Disease virus, Zika virus, Usutu virus, Wesselsbron and Omsk Hemorrhagic Fever virus. The sequence of the live, infectious, attenuated Flavivirus may be preceded by a sequence encoding a part of a flavivirus capsid protein comprising, consisting essentially of or consisting of the N-terminal part of the flavivirus Capsid protein, as described in International patent application WO2014174078, which is incorporated herein by reference.

In particular embodiments, the polynucleotide sequence encoding the chimeric virus comprises at the 5′ end consecutively, the 5′ end of the sequence encoding the core protein, the sequence encoding the Spike protein or part thereof, and the sequence encoding the core protein of the flavivirus.

In particular embodiments, the sequence of the live, infectious, attenuated Flavivirus is preceded by a sequence encoding a part of a flavivirus capsid protein comprising, consisting essentially of or consisting of the N-terminal part of the flavivirus Capsid protein, the nucleotide sequence encoding at least part of the Spike protein and a nucleotide encoding a 2A cleaving peptide. The person skilled in the art will understand that in such embodiment, the start codon (i.e. the first three nucleotides) of the sequence of the live, infectious, attenuated Flavivirus is deleted.

In particular embodiments, the polynucleotide sequence as taught herein comprises consecutively a nucleotide sequence encoding the N-terminal part of the capsid protein of the flavivirus, the nucleotide sequence encoding the at least part of the coronavirus Spike protein, a nucleotide encoding a 2A cleaving peptide and the nucleotide sequence of the live, infectious, attenuated Flavivirus.

In particular embodiments, the N-terminal part of the capsid protein of the flavivirus comprises the first 21 N-terminal amino acids of the capsid protein of the flavivirus. For example, the N-terminal part of the capsid protein of the flavivirus comprises, consists essentially of, or consist of the amino acid sequence MSGRKAQGKTLGVNMVRRGVR (SEQ ID NO: 25).

In particular embodiments, the N-terminal part of the capsid protein of the flavivirus is encoded by a nucleotide sequence 5′-ATGTCTGGTCGTAAAGCTCAGGGAAAAACCCTGGGCGTCAATATGGTACGACGAG GAGTTCGC-3′ (SEQ ID NO: 26). As described above, in embodiments wherein the polynucleotide encoding the Coronavirus Spike antigen is inserted prior to the C core of the flavirus, a sequence encoding a cleavage protein can be inserted 3′ of the sequence encoding the Spike protein. An efficient cleaving peptide is the Thosea asigna virus 2A peptide (T2A) [Donnelly et al. (2001) J Gen Virol 82, 1027-1041], the use of this peptide also overcomes the need to include a further ubiquitin cleavage sequence. The T2A peptide may have an amino acid sequence EGRGSLL TCGDVEENPGP (SEQ ID NO: 27).

Apart from Thosea asigna, other viral 2A peptides can be used in the compounds and methods of the present invention. Examples hereof are described in e.g. Chng et al. (2015) MAbs 7, 403-412, namely APVKQTLNFDLLKLAGDVESNPGP (SEQ ID NO: 28) of foot-and mouth disease virus, ATNFSLLKQAGDVEENPGP (SEQ ID NO: 29) of porcine teschovirus-1, and QCTNYALLKLAGDVESNPGP (SEQ ID NO: 30) from equine rhinitis A virus. These peptides have a conserved LxxxGDVExNPGP motif (SEQ ID NO: 31), wherein X can be any amino acid. Peptides with this consensus sequence can be used in the compounds of the present invention. Other suitable examples of viral 2A cleavage peptides represented by the consensus sequence DXEXNPGP (SEQ ID NO: 32) are disclosed in Souza-Moreira et al. (2018) FEMS Yeast Res. August 1, wherein X can be any amino acid. Further suitable examples of 2A cleavage peptides from as well picornaviruses as from insect viruses, type C rotaviruses, trypanosome and bacteria (T. maritima) are disclosed in Donnelly (2001) J Gen Virol. 82, 1027-1041.

As described above, the viral fusion constructs may further contain a repeat of the N-terminal part of the Capsid protein. The repeat of the N-terminal part of the Capsid protein may be present prior to the at least part of the Spike protein. In the present invention the repeat may have the same amino acid sequence but the DNA sequence may have been modified to include synonymous codons, resulting in a maximally −75% nucleotide sequence identity over the 21 codons used [herein codon 1 is the start ATG]. As demonstrated by Samsa et al. (2012) J. Virol. 2012 86, 1046-1058 the Capsid N-terminal part may be not limited to the 21 AA Capsid N terminal part, and may comprise for example an additional 5, 10, 15, 20 or 25 amino acids. Prior art only mutated cis-acting RNA structural elements from the repeat [Stoyanov (2010) Vaccine 28, 4644-4652]. Such approach thus also abolishes any possibility for homologous recombination, which leads to an extraordinary stable viral fusion construct.

In typical embodiment, the nucleotide sequence encoding the N-terminal part of the capsid protein, which is located 5′ of the sequence encoding the epitope or antigen (e.g. the at least part of the Spike protein of the coronavirus) is identical to the sequence of the virus used for the generation of the construct. The mutations which are typically introduced to avoid recombination are in such embodiment introduced in the nucleotide sequence encoding the N-terminal part of the capsid protein, which is located 3′ of the sequence encoding the epitope or antigen (e.g. the at least part of the Spike protein of the coronavirus).

Furthermore in the repeat of the C gene encoding the Capsid, the sequence only starts from the second codon, which likely affects cleavage from T2A; T2A cleavage is favored in the constructs of the present invention because the amino acid (aa) C-terminally of the T2A ‘cleavage’ site (NPG/P) [SEQ ID NO: 33] is a small amino acid, namely serine (NPG/PS) [SEQ ID NO: 34] or alternatively Gly, Ala, or Thr instead of the start methionine in the original Capsid protein.

Further also codon-optimized cDNAs may be used for the antigens that are cloned flavivirus constructs. Accordingly, in particular embodiments, the nucleotide sequence of the live, infectious, attenuated Flavivirus and/or the sequence encoding the at least part of the Spike protein of the coronavirus may be codon-optimized for expression in a host cell.

Overall, one or more of the above modifications minimize the replicative burden of inserting extra ‘cargo’ in the vector that would otherwise unavoidably pose on a fitness cost on YFV replication.

In particular embodiments, the sequence encoding at least part of the coronavirus Spike protein is inserted in the E/NS1 boundary of the flavivirus. In other words, in particular embodiments, the sequence encoding at least part of the Spike protein is inserted in between or located in between the nucleotide sequence encoding the envelope protein of the flavivirus and the sequence encoding the NS1 protein of the flavivirus. In other words, in particular embodiments, in the polynucleotide as taught herein, the nucleotide sequence encoding the S1 and S2 subunit of the coronavirus Spike protein is located 3′ of the nucleotide sequences encoding the envelope protein of the flavivirus and 5′ of the nucleotide sequences encoding the NS1 protein of the flavivirus.

In particular embodiments, in the polynucleotide as taught herein, the sequence encoding at least part of the Spike protein is located 3′ (downstream) of the nucleotide sequences encoding the capsid protein, the precursor membrane protein and the envelope protein of the flavivirus and 5′ (upstream) of the nucleotide sequences encoding the NS1, NS2A, NS2B, NS3, NS4A, NS4B, and NS5 proteins of the flavivirus.

In embodiments wherein the Spike protein or subunits thereof are inserted in the E/NS1 boundary, the constructs of the present invention allow a proper presentation of the encoded insert into the ER lumen and proteolytic processing. For this purpose the sequence encoding the signal peptide of the antigen (e.g. the sequence encoding the at least part of the Spike protein of the coronavirus) may be, and preferably is, partially or entirely removed and replaced by a sequence encoding the 9 amino acids of the NS1 protein of the flavivirus protein. For example, the 9 amino acids of the NS1 protein of the flavivirus may be DQGCAINFG (SEQ ID NO: 35) and may be encoded by a nucleotide sequence GACCAGGGCTGCGCGATAAATTTCGGT (SEQ ID NO: 36). Depending on the presence or absence of a transmembrane sequence in the antigen, the TM sequence of the antigen can be deleted and replaced by a flaviviral TM sequence, or one or more an additional TM membrane encoding sequences are inserted (or located) 3′ of the sequence encoding the antigen.

In particular embodiments, a sequence encoding a transmembrane (TM) domain of a further flavivirus is located 3′ (downstream) of the sequence encoding at least part of the Spike protein, and 5′ (upstream) of the NS1 region of the NS1-NS5 region of the flavivirus. Or in other words, in particular embodiments, a sequence encoding a transmembrane (TM) domain of a further flavivirus is located 3′ (downstream) of the sequence encoding at least part of the Spike protein, and 5′ (upstream) of the sequence encoding the NS1 protein.

In particular embodiments, the TM domain of a further flavivirus is a West Nile virus transmembrane domain 2 (WNV-TM2).

In particular embodiments, the WNV-TM2 comprises a nucleic acid sequence AGGTCAATTGCTATGACGTTTCTTGCGGTTGGAGGAGTTTTGCTCTTCCTTTCGGTC AACGTCCATGCT (SEQ ID NO: 37).

In particular embodiments, two TM domains of a further flavivirus are located 3′ of the sequence encoding the Spike protein S1 subunit, and 5′ of the NS-NS5 region. In other words, in particular embodiments, two sequences encoding a TM domain of a further flavivirus is located 3′ (downstream) of the sequence encoding at least the part of the coronavirus Spike protein, and 5′ (upstream) of the sequence encoding the NS1 protein.

In particular embodiments, the polynucleotide as taught herein comprises 5′ (upstream), and preferably immediately 5′ (upstream), to the sequence encoding the Spike protein or part thereof, a sequence encoding an NS1 signal peptide.

In particular embodiments, said NS1 signal peptide comprises a nucleic acid sequence GACCAGGGCTGCGCGATAAATTTCGGT (SEQ ID NO: 38).

Accordingly, in particular embodiments, if the first 39 nucleotides of the signal peptide of the Spike protein of the coronavirus are deleted, the polynucleotide as taught herein may comprise 5′ (upstream), and preferably immediately 5, to the sequence encoding the Spike protein or part thereof, a nucleotide sequence GACCAGGGCTGCGCGATAAATTTCGGTCAATGT (SEQ ID NO: 39), wherein the NS1 signal peptide of the NS1 signal peptide is indicated in bold and a 2 amino acid signal sequence is underlined.

Present inventors further have found that it is particular advantageous that:
the nucleotide sequence encoding at least part of the coronavirus Spike protein is inserted (is located) in the E/NS1 boundary of the flavivirus;
the nucleotide sequence encoding at least part of the coronavirus Spike protein does not comprise the nucleotide sequence encoding the signal peptide or part of the signal peptide of the coronavirus Spike protein, preferably wherein the nucleotide sequence encoding at least part of a coronavirus Spike protein does not comprise the first 39 nucleotides of the nucleotide sequence encoding the signal peptide of the coronavirus Spike protein;
a nucleotide sequence encoding a transmembrane (TM) domain of a further flavivirus is located 3′ of the nucleotide sequence encoding at least part of the coronavirus Spike protein, and 5′ of the NS1 region of the NS1-NS5 region, preferably wherein the TM domain of a further flavivirus is a West Nile virus transmembrane domain 2 (WNV-TM2); and/or
the polynucleotide comprises 5′ to the nucleotide sequence encoding at least part of the coronavirus Spike protein, a sequence encoding an NS1 signal peptide.

All of these particular advantageous features are present in the “YF-S0” vaccine as described elsewhere in the present specification.

Accordingly, in particular embodiments,
the nucleotide encoding at least part of the coronavirus Spike protein encodes the S1 and the S2 subunit of the coronavirus Spike protein; preferably the nucleotide sequence encoding the S1/S2 cleavage site is mutated, thereby preventing proteolytic processing of S protein in the S1 and S2 subunits;
the nucleotide sequence encoding at least part of the coronavirus Spike protein is inserted (is located) in the E/NS1 boundary of the flavivirus;
the nucleotide sequence encoding at least part of the coronavirus Spike protein does not comprise the first 39 nucleotides of the nucleotide sequence encoding the signal peptide of the coronavirus Spike protein;
a nucleotide sequence encoding a transmembrane (TM) domain of a further flavivirus is located 3′ of the nucleotide sequence encoding at least part of the coronavirus Spike protein, and 5′ of the NS1 region of the NS1-NS5 region, preferably wherein the TM domain of a further flavivirus is a West Nile virus transmembrane domain 2 (WNV-TM2); and
the polynucleotide comprises 5′ to the nucleotide sequence encoding at least part of the coronavirus Spike protein, a sequence encoding an NS1 signal peptide, preferably an NS1 signal peptide as defined by SEQ ID NO: 38.

In particular embodiments, the polynucleotide as taught herein comprises the sequence as defined by SEQ ID NO: 93 or 94, preferably SEQ ID NO: 94, or comprising a sequence encoding an amino acid sequence as defined by SEQ ID NO: 95 or 96, preferably SEQ ID NO: 95.

In particular embodiments, the polynucleotide as taught herein (i.e. the polynucleotide encoding the chimeric virus), comprises, consists essentially of, or consists of a sequence as defined by SEQ ID NO: 3, SEQ ID NO: 5, SEQ ID NO: 7, SEQ ID NO: 9, SEQ ID NO: 11 or SEQ ID NO: 13. In preferred embodiments, the polynucleotide as taught herein (i.e. the polynucleotide encoding the chimeric virus), comprises, consists essentially of, or consists of a sequence as defined by SEQ ID NO: 3, SEQ ID NO: 5 or SEQ ID NO: 7, preferably by SEQ ID NO: 5.

A further aspect provides an expression cassette, such as a viral expression cassette, comprising the polynucleotide sequence as taught herein.

A further aspect provides a vector comprising the expression cassette or the polynucleotide sequence as taught herein.

The propagation of the chimeric constructs prior to attenuation, as well as the cDNA of a construct after attenuation requires an error proof replication of the construct. The use of Bacterial Artificial Chromosomes, and especially the use of inducible BACS as disclosed by the present inventors in International patent application WO2014174078, and which is incorporated herein by reference, is particularly suitable for high yield, high quality amplification of cDNA of RNA viruses such as chimeric constructs of the present invention.

Accordingly, in particular embodiments, the vector comprising the expression cassette or the polynucleotide sequence as taught herein may be a BAC.

A BAC as described in this publication may comprise:
an inducible bacterial ori sequence for amplification of said BAC to more than 10 copies per bacterial cell, and
a viral expression cassette comprising a cDNA of the RNA virus genome and comprising cis-regulatory elements for transcription of said viral cDNA in mammalian cells and for processing of the transcribed RNA into infectious RNA virus.

As is the case in the present invention the RNA virus genome is a chimeric viral cDNA construct of two virus genomes.

In these BACS, the viral expression cassette comprises a cDNA of a positive-strand RNA virus genome, an typically
a RNA polymerase driven promoter preceding the 5′ end of said cDNA for initiating the transcription of said cDNA, and
an element for RNA self-cleaving following the 3′ end of said cDNA for cleaving the RNA transcript of said viral cDNA at a set position.

The BAC may further comprise a yeast autonomously replicating sequence for shuttling to and maintaining said bacterial artificial chromosome in yeast. An example of a yeast ori sequence is the 2μ plasmid origin or the ARS1 (autonomously replicating sequence 1) or functionally homologous derivatives thereof.

The RNA polymerase driven promoter of this aspect of the invention can be an RNA polymerase II promoter, such as Cytomegalovirus Immediate Early (CMV-IE) promoter, or the Simian virus 40 promoter or functionally homologous derivatives thereof.

The RNA polymerase driven promoter can equally be an RNA polymerase I or III promoter.

The BAC may also comprise an element for RNA self-cleaving such as the cDNA of the genomic ribozyme of hepatitis delta virus or functionally homologous RNA elements.

A further aspect provides a chimeric live, infectious, attenuated Flavivirus encoded by the polynucleotide sequence as taught herein.

In particular embodiments, the chimeric live, infectious, attenuated Flavivirus comprises, consists essentially of, or consists of an amino acid sequence as defined by SEQ ID NO: 4, SEQ ID NO: 6, SEQ ID NO: 8, SEQ ID NO: 10, SEQ ID NO: 12 or SEQ ID NO: 14, preferably SEQ ID NO: 4, SEQ ID NO: 6 or SEQ ID NO: 8, more preferably SEQ ID NO: 4.

A further aspect provides a pharmaceutical composition comprising the polynucleotide as taught herein or the chimeric virus as taught herein, and a pharmaceutically acceptable carrier.

the expression vector as taught herein, and a pharmaceutically acceptable carrier.

The term “pharmaceutically acceptable” as used herein is consistent with the art and means compatible with the other ingredients of a pharmaceutical composition and not deleterious to the recipient thereof.

In particular embodiments, the pharmaceutical composition is a vaccine.

The formulation of DNA into a vaccine preparation is known in the art and is described in detail in for example chapter 6 to 10 of “DNA Vaccines” Methods in Molecular Medicine Vol 127, (2006) Springer Saltzman, Shen and Brandsma (Eds.) Humana Press. Totoma, N.J. and in chapter 61 Alternative vaccine delivery methods, Pages 1200-1231, of Vaccines (6th Edition) (2013) (Plotkin et al. Eds.). Details on acceptable carrier, diluents, excipient and adjuvant suitable in the preparation of DNA vaccines can also be found in WO2005042014, as indicated below.

“Acceptable carrier, diluent or excipient” refers to an additional substance that is acceptable for use in human and/or veterinary medicine, with particular regard to immunotherapy.

By way of example, an acceptable carrier, diluent or excipient may be a solid or liquid filler, diluent or encapsulating substance that may be safely used in systemic or topic administration. Depending upon the particular route of administration, a variety of carriers, well known in the art may be used. These carriers may be selected from a group including sugars, starches, cellulose and its derivatives, malt, gelatine, talc, calcium sulphate and carbonates, vegetable oils, synthetic oils, polyols, alginic acid, phosphate buffered solutions, emulsifiers, isotonic saline and salts such as mineral acid salts including hydrochlorides, bromides and sulphates, organic acids such as acetates, propionates and malonates and pyrogen-free water.

A useful reference describing pharmaceutically acceptable carriers, diluents and excipients is Remington's Pharmaceutical Sciences (Mack Publishing Co. N. J. USA, (1991)) which is incorporated herein by reference.

Any safe route of administration may be employed for providing a patient with the DNA vaccine. For example, oral, rectal, parenteral, sublingual, buccal, intravenous, intra-articular, intra-muscular, intra-dermal, subcutaneous, inhalational, intraocular, intraperitoneal, intracerebroventricular, transdermal and the like may be employed. Intra-muscular and subcutaneous injection may be appropriate, for example, for administration of immunotherapeutic compositions, proteinaceous vaccines and nucleic acid vaccines. It is also contemplated that microparticle bombardment or electroporation may be particularly useful for delivery of nucleic acid vaccines.

Dosage forms include tablets, dispersions, suspensions, injections, solutions, syrups, troches, capsules, suppositories, aerosols, transdermal patches and the like. These dosage forms may also include injecting or implanting controlled releasing devices designed specifically for this purpose or other forms of implants modified to act additionally in this fashion. Controlled release of the therapeutic agent may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, the controlled release may be effected by using other polymer matrices, liposomes and/or microspheres.

DNA vaccines suitable for oral or parenteral administration may be presented as discrete units such as capsules, sachets or tablets each containing a pre-determined amount of plasmid DNA, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association one or more agents as described above with the carrier which constitutes one or more necessary ingredients. In general, the compositions are prepared by uniformly and intimately admixing the DNA plasmids with liquid carriers or finely divided solid carriers or both, and then, if necessary, shaping the product into the desired presentation.

The above compositions may be administered in a manner compatible with the dosage formulation, and in such amount as is effective. The dose administered to a patient, should be sufficient to effect a beneficial response in a patient over an appropriate period of time. The quantity of agent (s) to be administered may depend on the subject to be treated inclusive of the age, sex, weight and general health condition thereof, factors that will depend on the judgement of the practitioner.

Furthermore DNA vaccine may be delivered by bacterial transduction as using live-attenuated strain of Salmonella transformed with said DNA plasmids as exemplified by Darji et al. (2000) FEMS Immunol. Med. Microbiol. 27, 341-349 and Cicin-Sain et al. (2003) J. Virol. 77, 8249-8255 given as reference.

Typically the DNA vaccines are used for prophylactic or therapeutic immunisation of humans, but can for certain viruses also be applied on vertebrate animals (typically mammals, birds and fish) including domestic animals such as livestock and companion animals. The vaccination is envisaged of animals which are a live reservoir of viruses (zoonosis) such as monkeys, mice, rats, birds and bats.

In certain embodiments vaccines may include an adjuvant, i.e. one or more substances that enhances the immunogenicity and/or efficacy of a vaccine composition However, life vaccines may eventually be harmed by adjuvants that may stimulate innate immune response independent of viral replication. Non-limiting examples of suitable adjuvants include squalane and squalene (or other oils of animal origin); block copolymers; detergents such as Tween-80; Quill A, mineral oils such as Drakeol or Marcol, vegetable oils such as peanut oil; Corynebacterium-derived adjuvants such as Corynebacterium parvum; Propionibacterium-derived adjuvants such as Propionibacterium acne; Mycobacterium bovis (Bacille Calmette and Guerin or BCG); interleukins such as interleukin 2 and interleukin 12; monokines such as interleukin 1; tumour necrosis factor; interferons such as gamma interferon; combinations such as saponin-aluminium hydroxide or Quil-A aluminium hydroxide; liposomes; ISCOMt) and ISCOMATRIX (B) adjuvant; mycobacterial cell wall extract; synthetic glycopeptides such as muramyl dipeptides or other derivatives; Avridine; Lipid A derivatives; dextran sulfate; DEAE-Dextran or with aluminium phosphate; carboxypolymethylene such as Carbopol'EMA; acrylic copolymer emulsions such as Neocryl A640; vaccinia or animal poxvirus proteins; sub-viral particle adjuvants such as cholera toxin, or mixtures thereof.

A further aspect provides an in vitro method of preparing a chimeric virus as taught herein.

A further aspect provides an in vitro method of preparing a vaccine against a coronavirus infection comprising a chimeric virus or a polynucleotide as taught herein.

A further aspect provides an in vitro method of preparing a vaccine against a coronavirus infection, comprising the steps of:

a) providing a BAC which comprises:
an inducible bacterial ori sequence for amplification of said BAC to more than 10 copies per bacterial cell, and
a viral expression cassette comprising a cDNA of a chimeric virus comprising a polynucleotide as taught herein, and comprising cis-regulatory elements for transcription of said viral cDNA in mammalian cells and for processing of the transcribed RNA into infectious RNA virus,
b) transfecting mammalian cells with the BAC of step a) and passaging the infected cells,
c) validating replicated virus of the transfected cells of step b) for virulence and the capacity of generating antibodies and inducing protection against coronavirus infection,
d) cloning the virus validated in step c) into a vector, and formulating the vector into a vaccine formulation.

In particular embodiments, the vector is BAC, which comprises an inducible bacterial ori sequence for amplification of said BAC to more than 10 copies per bacterial cell.

A further aspect provides the polynucleotide as taught herein, the chimeric virus as taught herein, or the pharmaceutical composition as taught herein for use as a medicament, preferably wherein the medicament is a vaccine.

A further aspect provides the polynucleotide as taught herein, the chimeric virus as taught herein, or the pharmaceutical composition as taught herein for use in preventing a coronavirus infection, preferably a SARS-CoV-2 infection. In other words, provided herein is a method for preventing a coronavirus infection (e.g. a method of vaccinating against a coronavirus), preferably a SARS-CoV2 infection, in a subject comprising administering a prophylactically effective amount of the polynucleotide as taught herein, the chimeric virus as taught herein, or the pharmaceutical composition as taught herein. Except when noted, the terms “subject” or “patient” can be used interchangeably and refer to animals, preferably warm-blooded animals, more preferably vertebrates, even more preferably mammals, still more preferably primates, and specifically includes human patients and non-human mammals and primates. Preferred subjects are human subjects.

Present inventors have shown that a single dose of the polynucleotide sequence as taught herein is sufficient

In particular embodiments, a single dose of the the polynucleotide as taught herein, the chimeric virus as taught herein, or the pharmaceutical composition as taught herein is administered to the subject.

In particular embodiments, the single dose comprises, consists essentially of or consists of from between 10⁴ to 10⁶ PFU, such as about 10⁵, PFU of the chimeric virus as taught herein.

The present application also provides aspects and embodiments as set forth in the following Statements:

Statement 1. A polynucleotide comprising a sequence of a live, infectious, attenuated Flavivirus wherein a nucleotide sequence encoding at least a part of a coronavirus Spike protein is inserted, such that a chimeric virus is expressed.
Statement 2. The polynucleotide according to statement 1, wherein the flavivirus is yellow fever virus.
Statement 3. The polynucleotide according to statement 1 or 2, wherein the flavivirus is YF17D.
Statement 4. The polynucleotide according to any one of statements 1 to 3, wherein the coronavirus is Covid 19.
Statement 5. The polynucleotide according to any one of statements 1 to 4, wherein the sequence encoding the signal peptide or part of the signal peptide of the Spike protein (between 1 and 42 nucleotides) is deleted.
Statement 6. The polynucleotide according to any one of statements 1 to 5, encoding the S1 and S2 subunit of spike protein.
Statement 7. The polynucleotide according to any one of statements 1 to 8, wherein the nucleotide sequence encoding the S1/S2 cleavage site mutated, thereby preventing proteolytic processing of S protein in S1 and S2 subunits.
Statement 8. The polynucleotide according to any one of statements 1 to 8, wherein the nucleotide sequence encoding the S2′ cleavage site is mutated, thereby preventing proteolytic processing.
Statement 9. The polynucleotide according to any one of statements 1 to 8, wherein the nucleotide sequence encodes the spike protein S2 subunit (i.e. the sequence encoding the S1 subunit is deleted).
Statement 10. The polynucleotide according to any one of statements 1 to 8, wherein the nucleotide sequence encodes the spike S1 subunit (i.e. the sequence encoding the S2 subunit is deleted).
Statement 11. The polynucleotide according to any one of statements 1 to 10, wherein the sequence encoding the Spike protein or apart thereof is inserted in the E/NS1 boundary of the flavivirus.
Statement 12. The polynucleotide according to statement 11, wherein a sequence encoding a transmembrane (TM) domain of a further flavivirus is located 3′ of the sequence encoding the Spike protein or part thereof, and 5′ of the NS1 region of the NS1-NS5 region.
Statement 13. The polynucleotide according to statement 11 or 12, comprising 5′ to the sequence encoding the Spike protein or part thereof, a sequence encoding an NS1 signal peptide
Statement 14. The polynucleotide according to any one of statements 11 to 13, wherein two TM domains of a further flavivirus are located 3′ of the sequence encoding the Spike protein S1 subunit, and 5′ of the NS1-NS5 region.
Statement 15. The polynucleotide according to any one of statements 1 to 10, wherein the nucleotide sequence encoding the chimeric virus comprises at the 5′ end consecutively, the 5′ end of the sequence encoding the core protein, the sequence encoding the Spike protein or part thereof, and the core protein of the flavivirus.
Statement 16. The polynucleotide according to statement 15, wherein the sequence encoding part of the spike protein is the S1 domain (ie the S2 domain is deleted).
Statement 17. The polynucleotide according to any one of statements 1 to 8, comprising a sequence selected from the group consisting of SEQ ID NO: 3, SEQ ID NO: 5, SEQ ID NO: 7, SEQ ID NO: 9, SEQ ID NO: 11 and SEQ ID NO: 13. If cloned in another backbone than YFV, the 5′ and 3′ of the above cited SEQ ID are modified into the sequence of the backbone.
Statement 18. The polynucleotide according to any one of the statements 1 to 17, which is a bacterial artificial chromosome.
Statement 19. A polynucleotide in accordance to any one of statement 1 to 18, for use as a medicament.
Statement 20. The polynucleotide for use as a medicament in accordance with statement 19, wherein the medicament is a vaccine.
Statement 21. A polynucleotide sequence in accordance to any one of statement 1 to 18, for use in the vaccination against a coronavirus.
Statement 22. A chimeric live, infectious, attenuated Flavivirus encoded by a nucleotide sequence according to any one of statement 1 to 18.
Statement 23. A chimeric virus in accordance to statement 22, for use as a medicament.
Statement 24. A chimeric virus in accordance to statement 22, for use in the prevention of a coronavirus infection.
Statement 25. A method of preparing a vaccine against a coronavirus infection, comprising the steps of:
a) providing a BAC which comprises:
an inducible bacterial ori sequence for amplification of said BAC to more than 10 copies per bacterial cell, and
a viral expression cassette comprising a cDNA of a chimeric virus according to any one of statements 1 to 17, and comprising cis-regulatory elements for transcription of said viral cDNA in mammalian cells and for processing of the transcribed RNA into infectious RNA virus,
b) transfecting mammalian cells with the BAC of step a) and passaging the infected cells,
c) validating replicated virus of the transfected cells of step b) for virulence and the capacity of generating antibodies and inducing protection against coronavirus infection,
d) cloning the virus validated in step c into a vector, and formulating the vector into a vaccine formulation.
Statement 26. The method according to statement 25, wherein the vector is BAC, which comprises an inducible bacterial ori sequence for amplification of said BAC to more than 10 copies per bacterial cell.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims.

The herein disclosed aspects and embodiments of the invention are further supported by the following non-limiting examples.

### EXAMPLES

### Example 1: Spike Gene Sequence Inserted Between YF-E/NS1

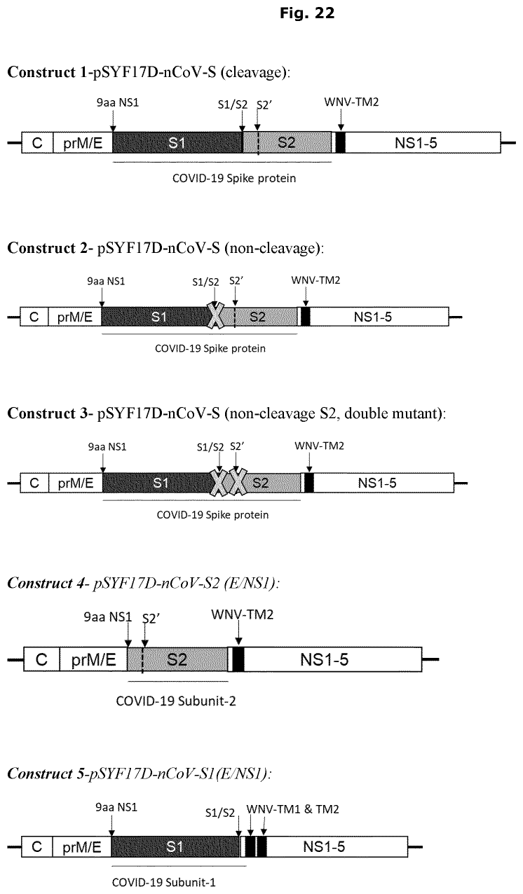

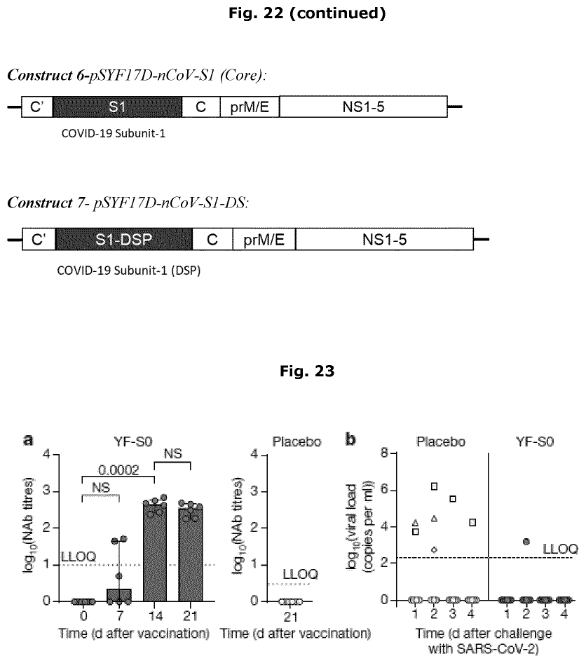

Construct 1—pSYF17D-nCoV-S(cleavage): (the COVID-19 spike with the first 13 aa from the signal peptide [SP] deleted and C-terminus fused to West Nile virus transmembrane domain 2 (WNV-TM2)). (FIG. 22) Construct 1 corresponds to “YF-S1/S2” as referred to in examples 8 and 9.

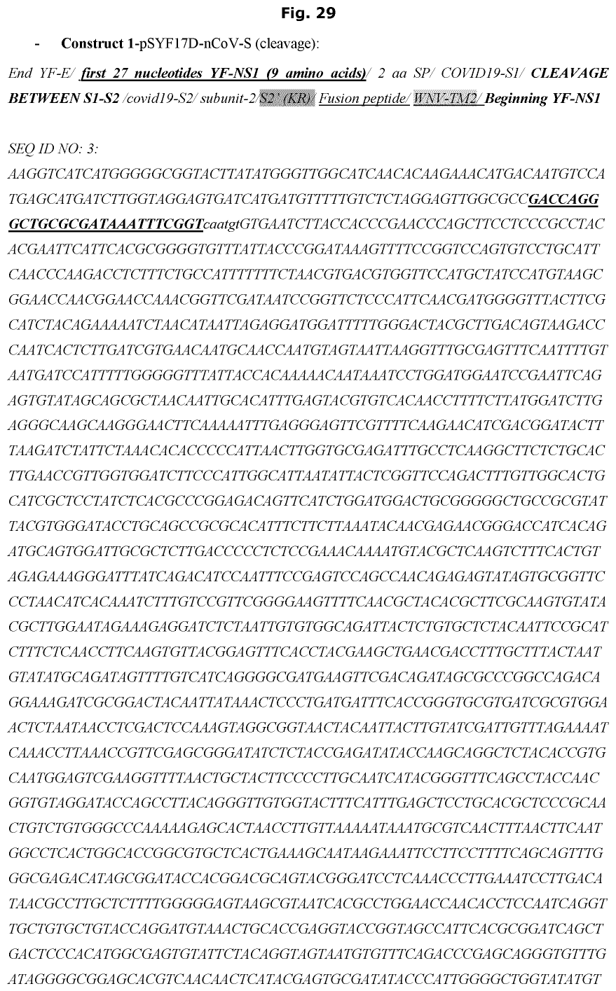

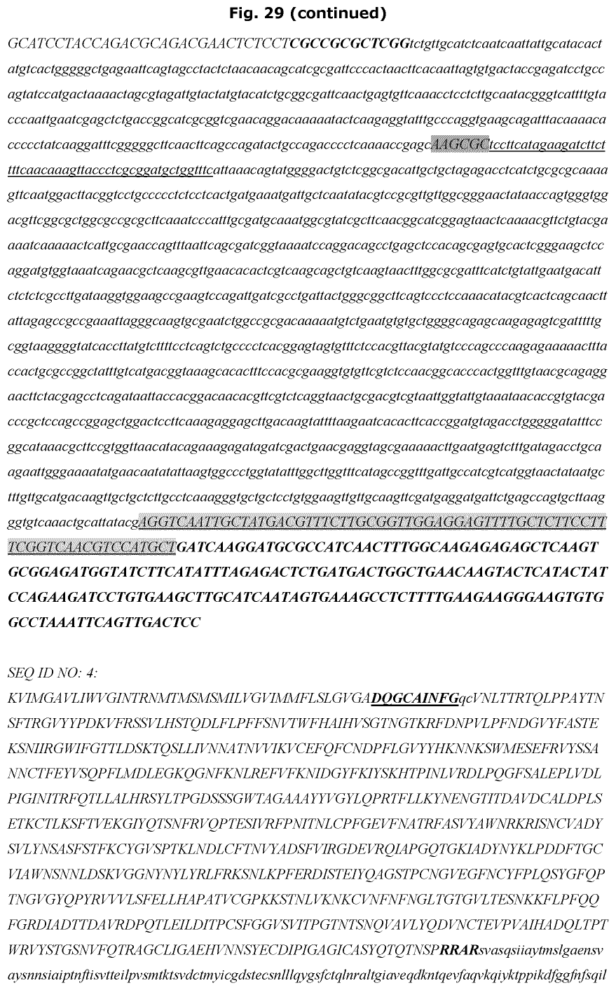

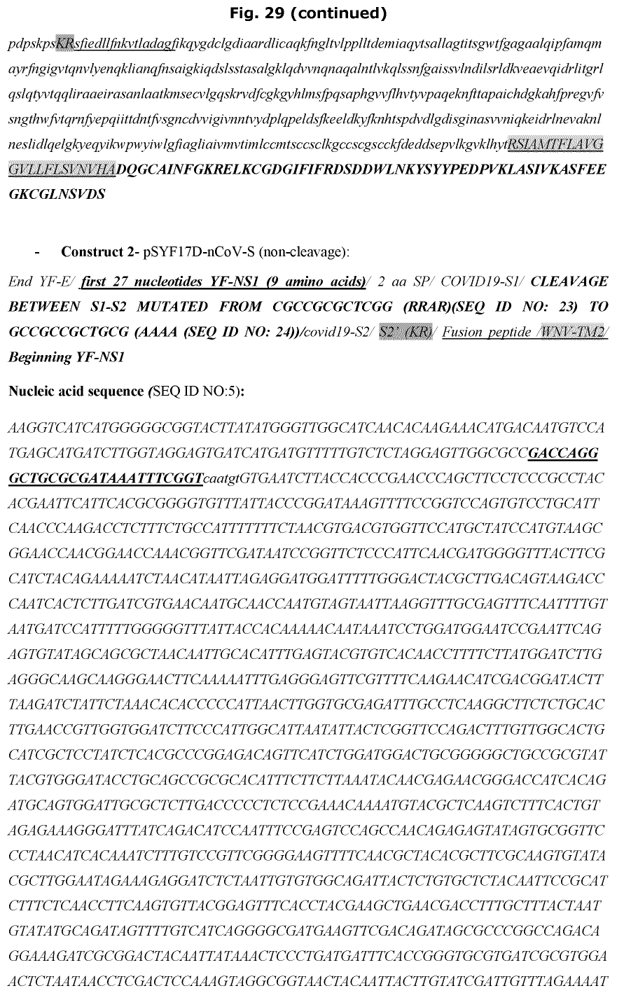

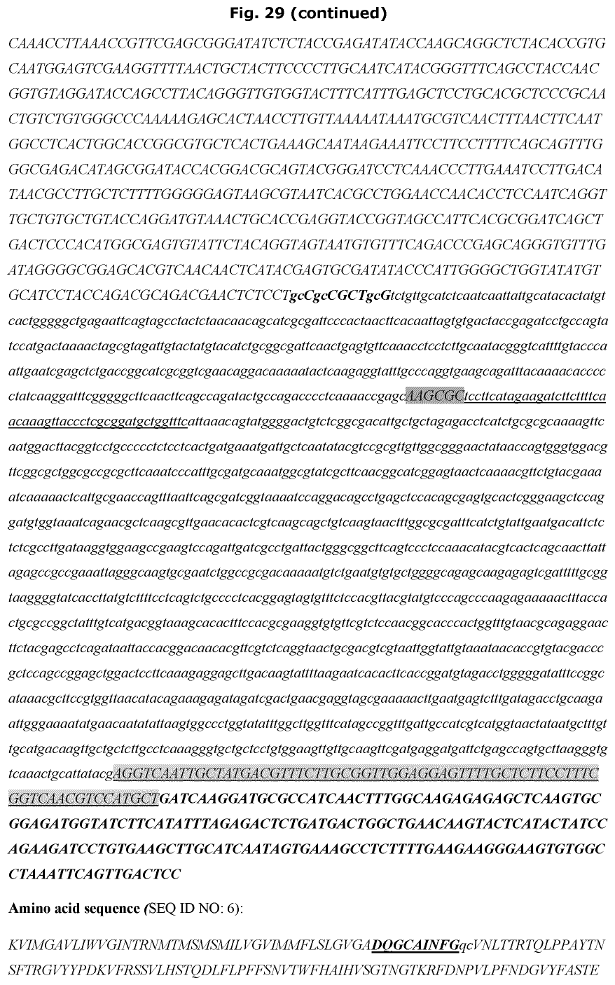

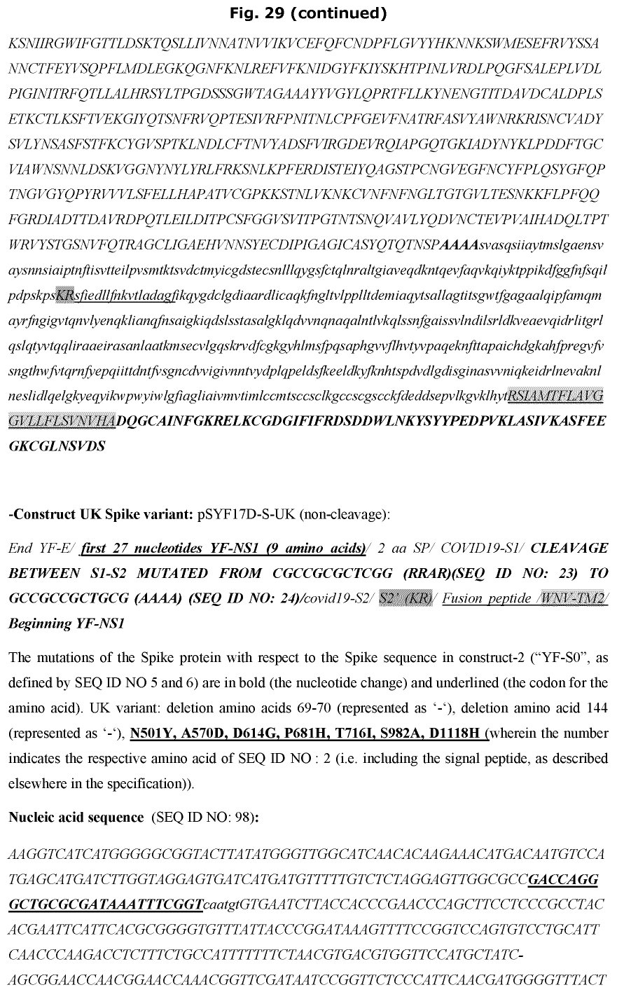

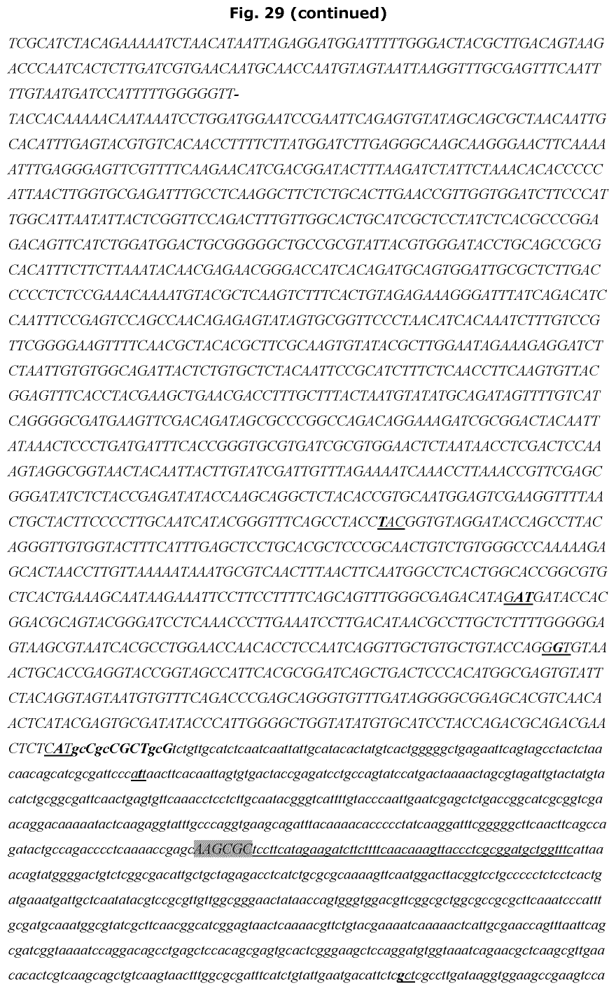

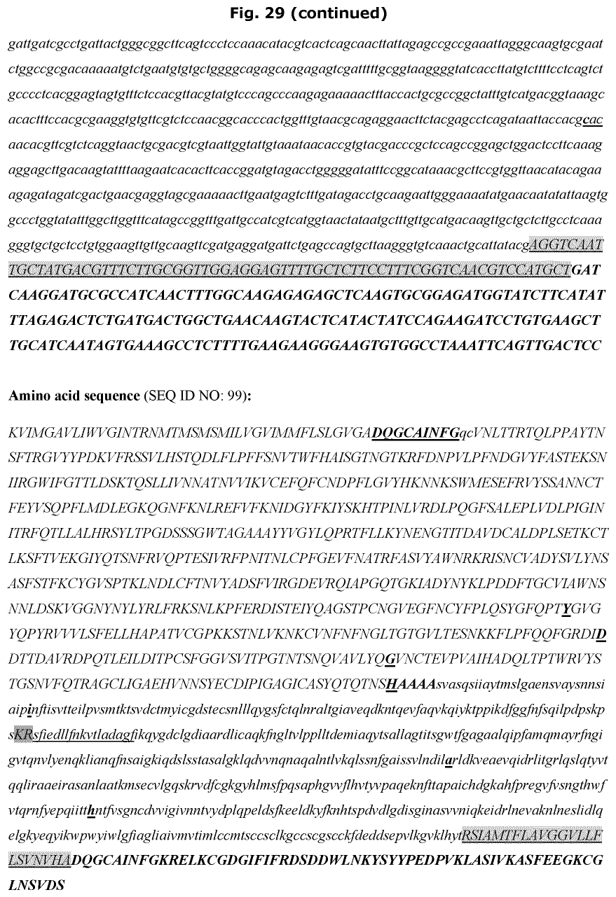

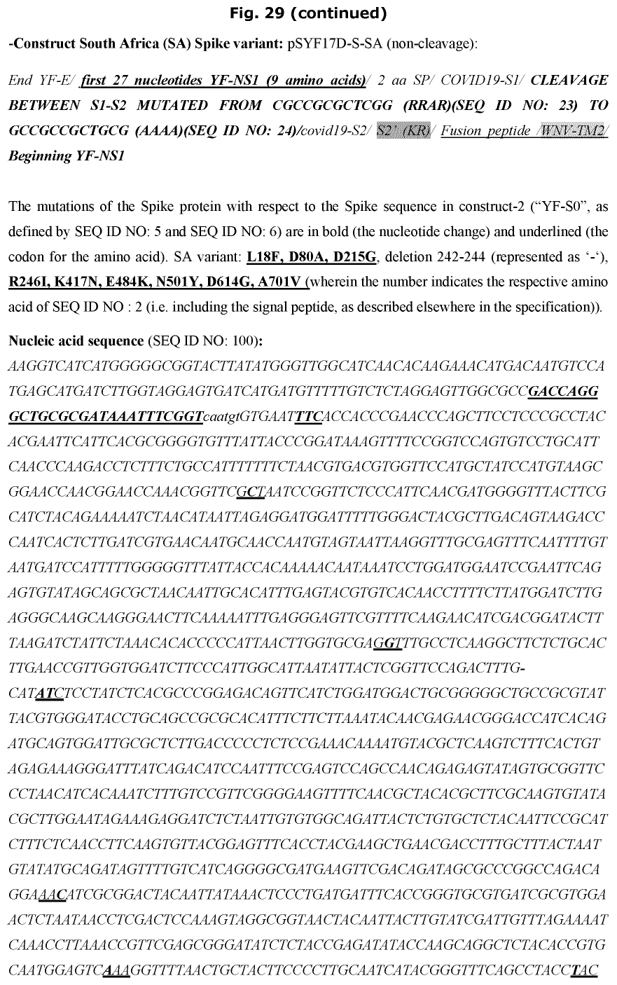

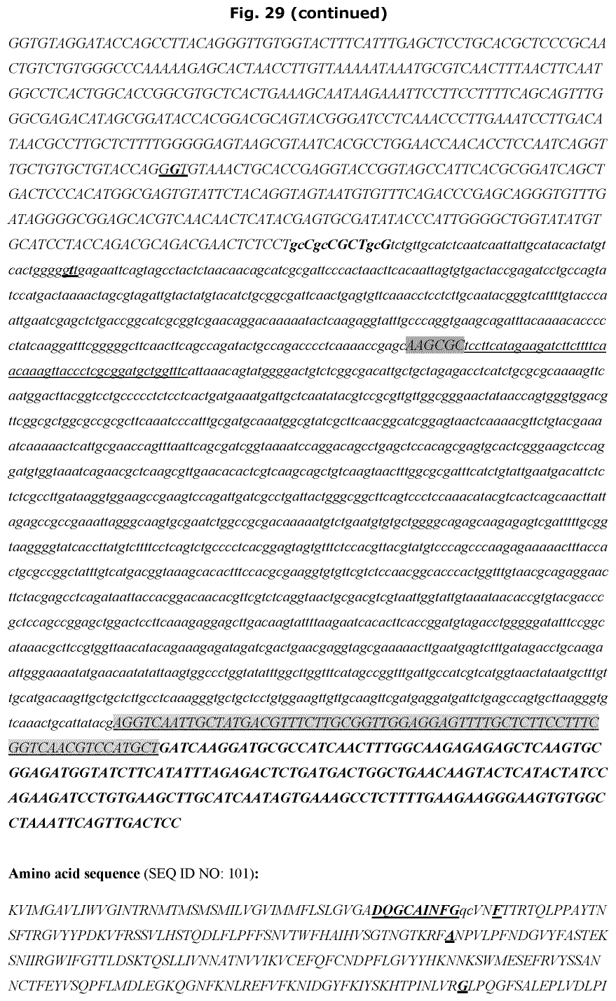

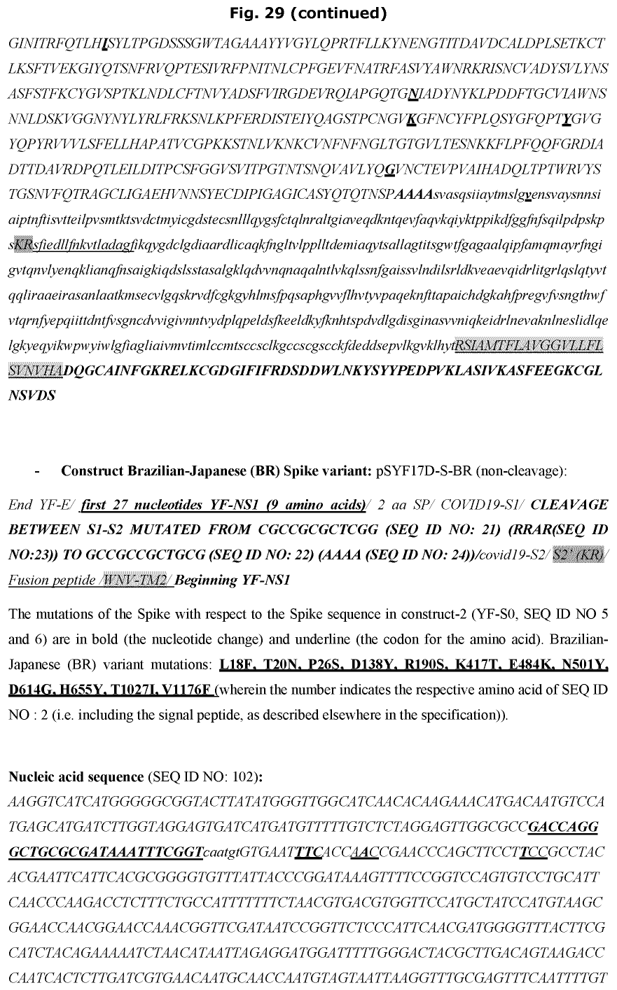

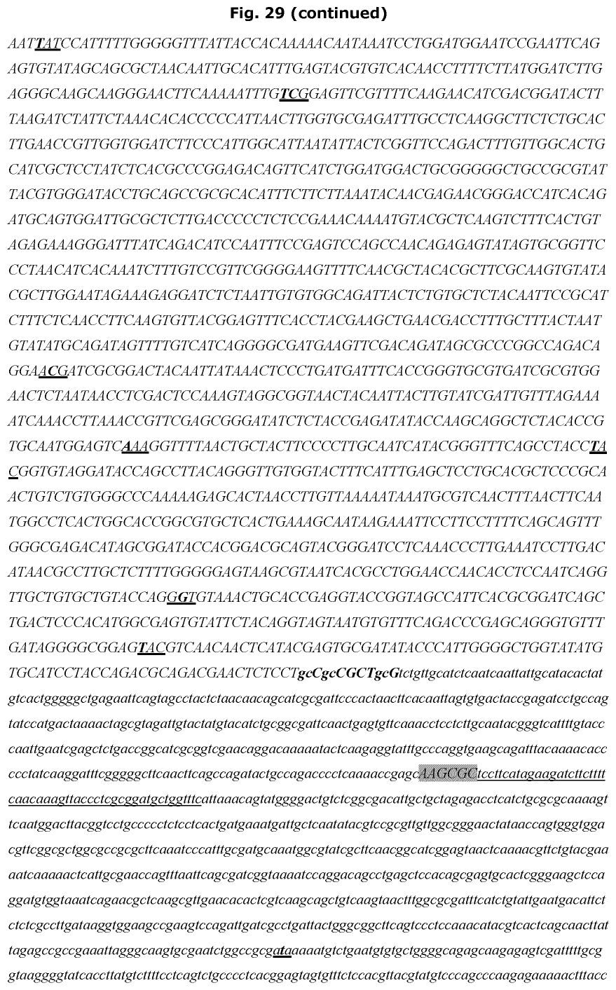

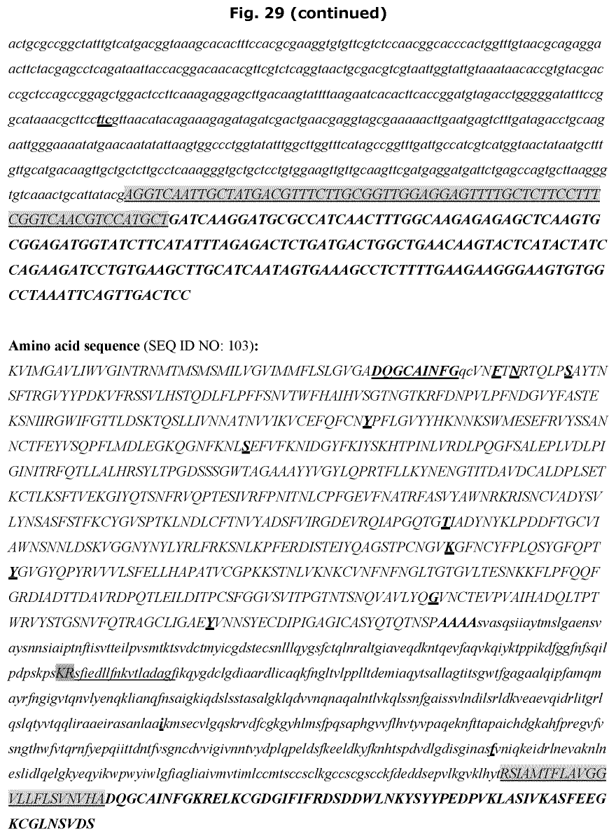

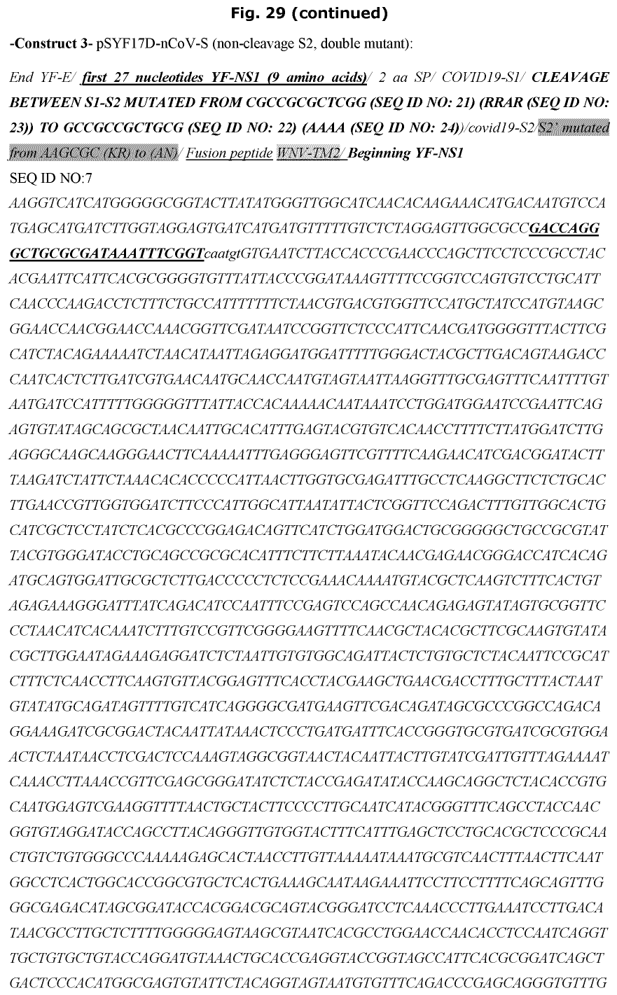

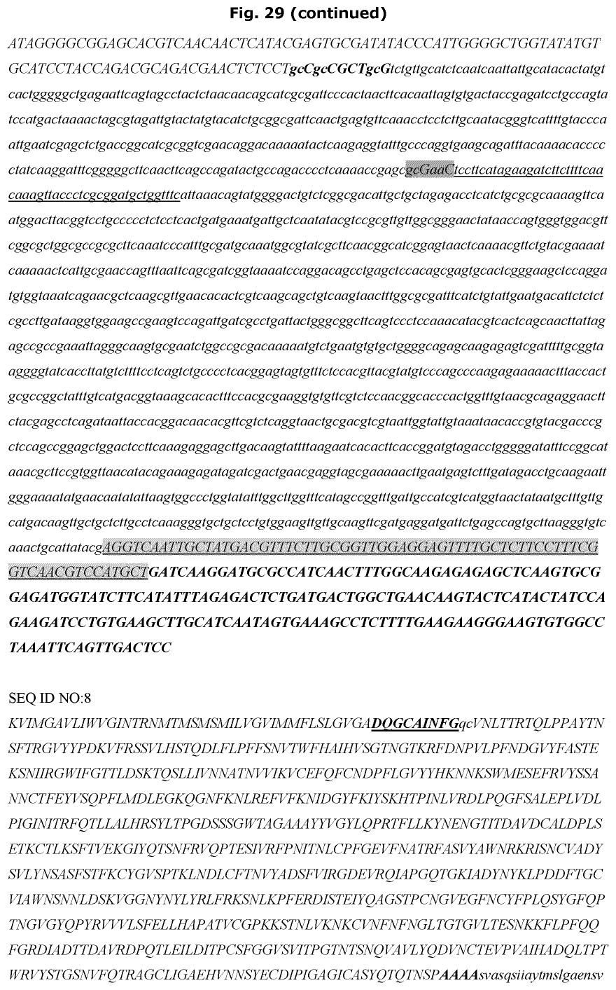

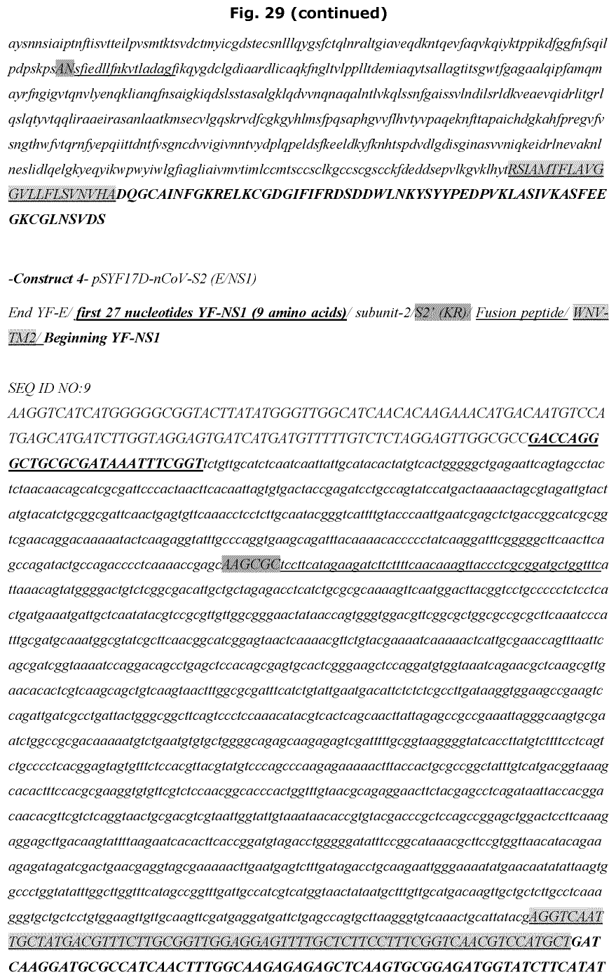

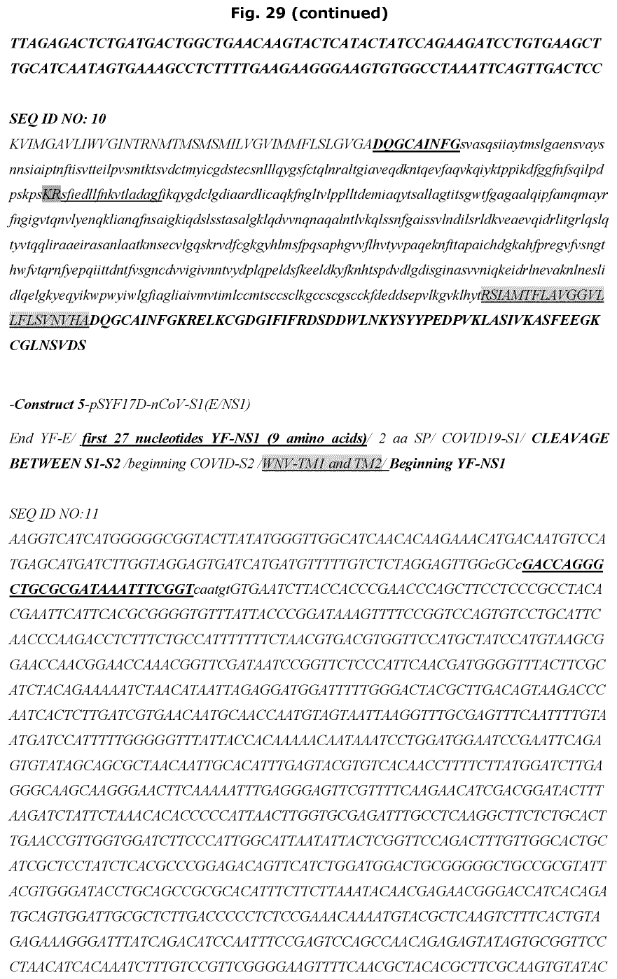

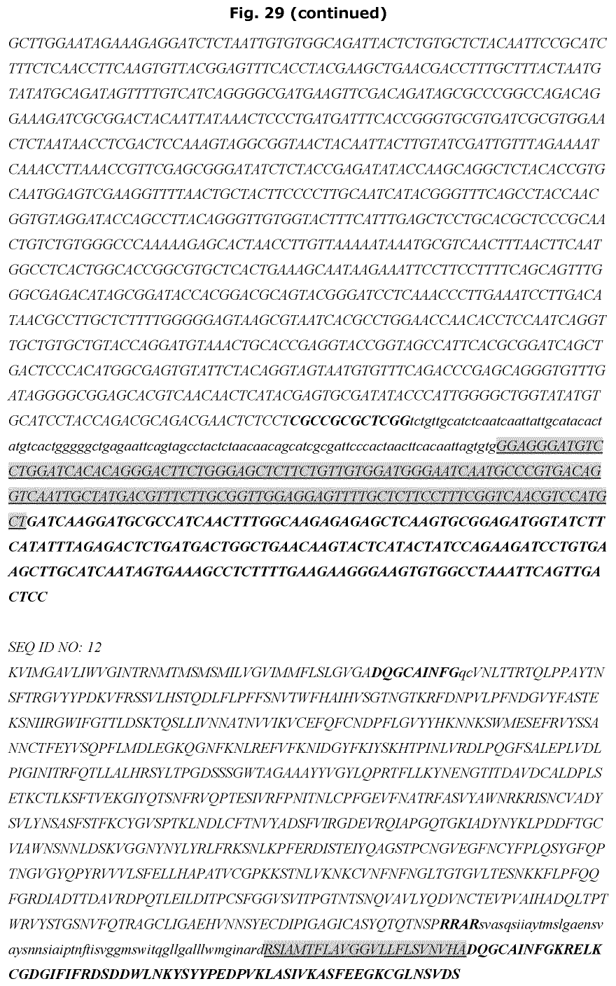

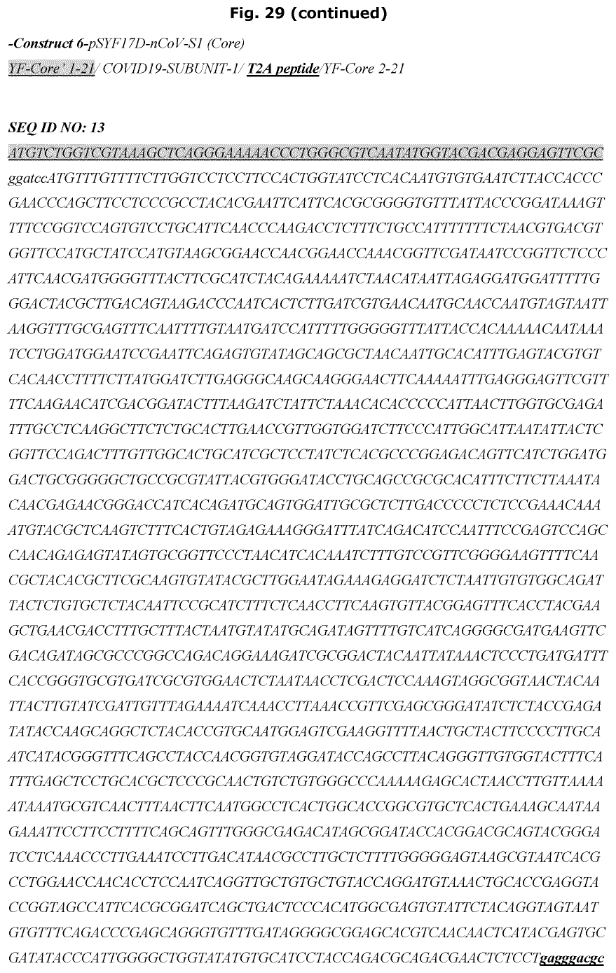

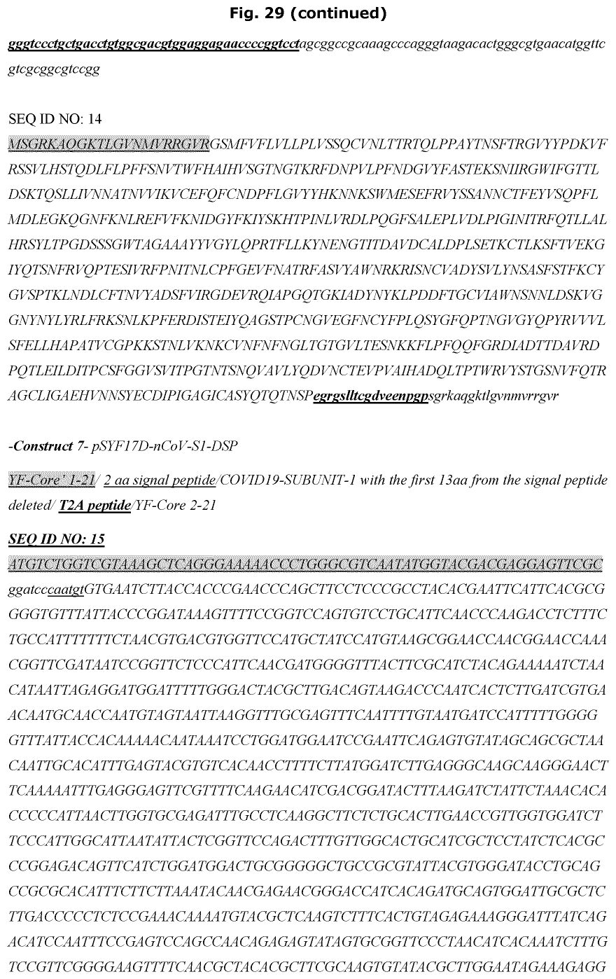

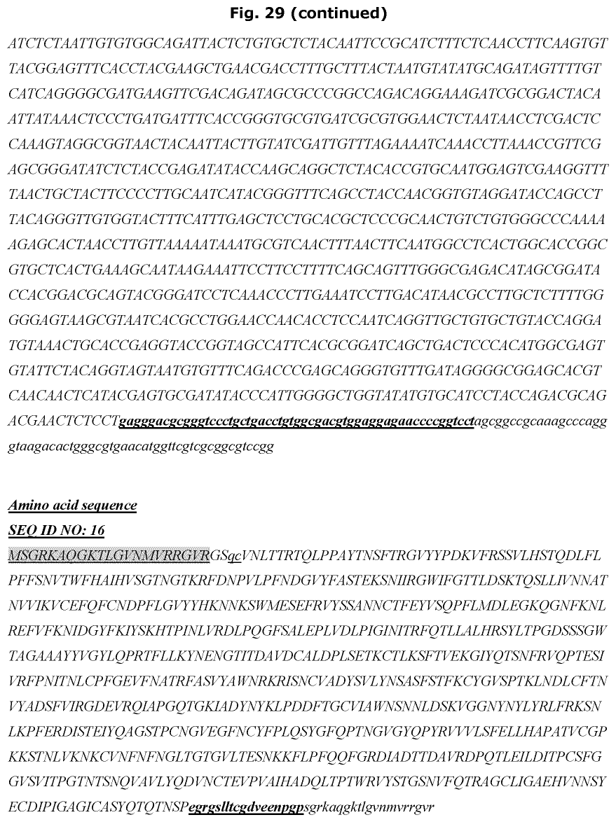

Annotations of nucleic acid (SEQ ID NO: 3) and amino acid sequence (SEQ ID NO: 4) as shown in FIG. 29: End YF-E/first 27 nucleotides YF-NS1 (9 amino acids)/2 aa SP/COVID19-S1/CLEAVAGE BETWEEN S1-S2/covid19-S2/subunit-2/S2′ (KR)/Fusion peptide/WNV-TM2/Beginning YF-NS1

### Example 2: Constructs with Spike Gene Sequence Inserted Between YF-E/NS1

—Construct 2—pSYF17D-nCoV-S(non-cleavage): (the COVID-19 spike with the first 13 aa from the signal peptide (SP) deleted, cleavage site S1/S2 mutated from RRAR (SEQ ID NO: 23) to AAAA (SEQ ID NO: 24) and C-terminus fused to West Nile virus transmembrane domain 2 (WNV-TM2)). (FIG. 22) Construct 2 corresponds to “YF-S0” as referred to in examples 8 and 9.

Annotations of nucleic acid (SEQ ID NO: 5) and amino acid sequence (SEQ ID NO: 6) as shown in FIG. 29: End YF-E/first 27 nucleotides YF-NS1 (9 amino acids)/2 aa SP/COVID19-S1/CLEAVAGE BETWEEN S1-S2 MUTATED FROM CGCCGCGCTCGG (RRAR)(SEQ ID NO: 23) TO GCCGCCGCTGCG (AAAA (SEQ ID NO: 24))/covid19-S2/S2′ (KR)/Fusion peptide/WNV-TM2/Beginning YF-NS1

—Construct UK Spike variant: pSYF17D-S-UK (non-cleavage): the spike protein from SARS-CoV2 UK variant (VOC 202012/01, B.1.1.7) with the first 13 aa from the signal peptide (SP) deleted, cleavage site S1/S2 mutated from RRAR (SEQ ID NO: 23) to AAAA (SEQ ID NO: 24) and C-terminus of the spike protein fused to West Nile virus transmembrane domain 2 (WNV-TM2)).

Annotations of nucleic acid (SEQ ID NO: 98) and amino acid sequence (SEQ ID NO: 99) as shown in FIG. 29: End YF-E/first 27 nucleotides YF-NS1 (9 amino acids)/2 aa SP/COVID19-S1/CLEAVAGE BETWEEN S1-S2 MUTATED FROM CGCCGCGCTCGG (RRAR)(SEQ ID NO: 23) TO GCCGCCGCTGCG (AAAA) (SEQ ID NO: 24)/covid19-S2/S2′ (KR)/Fusion peptide/WNV-17112/Beginning YF-NS1

The mutations of the Spike protein with respect to the Spike sequence in construct-2 (“YF-S0”, as defined by SEQ ID NO 5 and 6) are in bold (the nucleotide change) and underlined (the codon for the amino acid) in SEQ ID NO: 98 in FIG. 29. UK variant: deletion amino acids 69-70 (represented as ‘-’), deletion amino acid 144 (represented as ‘-’), N501Y, A570D, D614G, P68111, T716I, S982A, D1118H (wherein the number indicates the respective amino acid of SEQ ID NO: 2 (i.e. including the signal peptide, as described elsewhere in the specification)).

—Construct South Africa (SA) Spike variant: pSYF17D-S-SA (non-cleavage): the spike protein from South-Africa variant (VOC 501Y.V2, B. 1.351) with the first 13 aa from the signal peptide (SP) deleted, cleavage site S1/S2 mutated from RRAR (SEQ ID NO: 23) to AAAA (SEQ ID NO: 24) and C-terminus of the spike protein fused to West Nile virus transmembrane domain 2 (WNV-TM2)).

Annotations of nucleic acid (SEQ ID NO: 100) and amino acid sequence (SEQ ID NO: 101) as shown in FIG. 29: End YF-E/first 27 nucleotides YF-NS1 (9 amino acids)/2 aa SP/COVID19-S1/CLEAVAGE BETWEEN S1-S2 MUTATED FROM CGCCGCGCTCGG (RRAR)(SEQ ID NO: 23) TO GCCGCCGCTGCG (AAAA) (SEQ ID NO: 24)/covid19-S2/S2′ (KR)/Fusion peptide/WNV-TM2/Beginning YF-NS1

The mutations of the Spike protein with respect to the Spike sequence in construct-2 (“YF-S0”, as defined by SEQ ID NO: 5 and SEQ ID NO: 6) are in bold (the nucleotide change) and underlined (the codon for the amino acid) in SEQ ID NO: 100 in FIG. 29. SA variant: L18F, D80A D215G, deletion 242-244 (represented as ‘-’), R246I, K417N, E484K, N501Y, D614G, A701V (wherein the number indicates the respective amino acid of SEQ ID NO: 2 (i.e. including the signal peptide, as described elsewhere in the specification)).

—Construct Brazilian-Japanese (BR) Spike variant: pSYF17D-S-BR (non-cleavage): the spike protein from Brazilian-Japanese (B.1.1.248) variant with the first 13 aa from the signal peptide (SP) deleted, cleavage site S1/S2 mutated from RRAR (SEQ ID NO: 23) to AAAA (SEQ ID NO: 24) and C-terminus of the spike protein fused to West Nile virus transmembrane domain 2 (WNV-TM2)).

Annotations of nucleic acid (SEQ ID NO: 102) and amino acid sequence (SEQ ID NO: 103) shown in FIG. 29: End YF-E/first 27 nucleotides YF-NS1 (9 amino acids)/2 aa SP/COVID19-S1/CLEAVAGE BETWEEN S1-S2 MUTATED FROM CGCCGCGCTCGG (SEQ ID NO: 21) (RRAR(SEQ ID NO:23)) TO GCCGCCGCTGCG (SEQ ID NO: 22) (AAAA (SEQ ID NO: 24))/covid19-S2/S2′ (KR)/Fusion peptide/WNV-TM2/Beginning YF-NS1

The mutations of the Spike with respect to the Spike sequence in construct-2 (YF-S0, SEQ ID NO 5 and 6) are in bold (the nucleotide change) and underlined (the codon for the amino acid) in SEQ ID NO: 102 in FIG. 29. Brazilian-Japanese (BR) variant mutations: L18F, T20N, P26S, D138Y, R190S, K417T, E484K, N501Y, D614G, 11655Y, T1027I, V1176F (wherein the number indicates the respective amino acid of SEQ ID NO: 2 (i.e. including the signal peptide, as described elsewhere in the specification)).

### Example 3. Constructs with Spike Gene Sequence Inserted Between YF-E/NS1

—Construct 3—pSYF17D-nCoV-S(non-cleavage S2, double mutant): (the COVID-19 spike with the first 13 aa from the signal peptide (SP) deleted, cleavage site S1/S2 mutated from RRAR (SEQ ID NO: 23) to AAAA (SEQ ID NO: 24), second cleavage S2′ mutated from KR to AN and C-terminus fused to West Nile virus transmembrane domain 2 (WNV-TM2)). (FIG. 22)

Annotations of nucleic acid (SEQ ID NO: 7) and amino acid sequence (SEQ ID NO: 8) as shown in FIG. 29: End YF-E/first 27 nucleotides YF-NS1 (9 amino acids)/2 aa SP/COVID19-S1/CLEAVAGE BETWEEN S1-S2 MUTATED FROM CGCCGCGCTCGG (SEQ ID NO: 21) (RRAR (SEQ ID NO: 23)) TO GCCGCCGCTGCG (SEQ ID NO: 22) (AAAA (SEQ ID NO: 24))/covid19-S2/S2′ mutated from AAGCGC (KR) to (AN)/Fusion peptide WNV-TM2/Beginning YF-NS1

### Example 4: Constructs with Spike Gene Sequence S2 Subunit Inserted Between YF-E/NS1

—Construct 4—pSYF17D-nCoV-S2 (E/NS1) (COVID-19 spike subunit-2 inserted between YF17D-E/NS1, C-terminus fused to West Nile virus transmembrane domain 2 (WNV-TM2)). (FIG. 22)

Annotations of nucleic acid (SEQ ID NO: 9) and amino acid sequence (SEQ ID NO: 10) shown in FIG. 29: End YF-E/first 27 nucleotides YF-NS1 (9 amino acids)/subunit-2/S2′ (KR)/Fusion peptide/WNV-TM2/Beginning YF-NS1

### Example 5: Constructs with Spike Gene Sequence S1 Subunits Inserted Between YF-E/NS1

—Construct 5—pSYF17D-nCoV-S1(E/NS1) (COVID-19 spike subunit-1 inserted between YF17D-E/NS1, the first 13 aa from the signal peptide deleted and fused to WNV-TM1 and TM2). (FIG. 22)

Construct 5 corresponds to “YF-S1” as referred to in examples 8 and 9.

Annotations of nucleic acid (SEQ ID NO: 11) and amino acid sequence (SEQ ID NO: 12) as shown in FIG. 29: End YF-E/first 27 nucleotides YF-NS1 (9 amino acids)/2 aa SP/COVID19-S1/CLEAVAGE BETWEEN S1-S2/beginning COVID-S2/WNV TM1 and TM2/Beginning YF-NS1

### Example 6: S1 Subunit Gene Sequence Inserted in YF-Core

—Construct 6-pSYF17D-nCoV-S1 (Core) (COVID-19 spike subunit 1) (FIG. 22)

Annotations of nucleic acid (SEQ ID NO: 13) and amino acid sequence (SEQ ID NO: 14) as shown in FIG. 29: YF-Core′ 1-21/COVID19-SUBUNIT-1/T2A peptide/YF-Core 2-21

### Example 7S: Subunit Gene Sequence Inserted in YF-Core

—Construct 7—pSYF17D-nCoV-S1-DSP (COVID-19 spike subunit 1 with the first 13 aa from the signal peptide deleted) (FIG. 22)

Annotations of nucleic acid (SEQ ID NO: 15) and amino acid sequence (SEQ ID NO: 16) shown in FIG. 29: YF-Core′ 1-21/2 aa signal peptide/COVID19-SUBUNIT-1 with the first 13aa from the signal peptide deleted/T2A peptide/YF-Core 2-21

### Example 8: Assessment of Vaccine Safety, Immunogenicity and Efficacy of Constructs 1, 2 and 5 in Several Animal Models

### 8.1 Vaccine Design and Rationale

Protective immunity against SARS-CoV-2 and other coronaviruses is believed to depend on neutralizing antibodies (nAbs) targeting the viral Spike (S) protein^{3,4}. In particular, nAbs specific for the N-terminal S1 domain containing the Angiotensin Converting Enzyme 2 (ACE2) receptor binding domain (RBD) interfere with and have been shown to prevent viral infection in several animal models^{5,6}.

The live-attenuated YF17D vaccine is known for its outstanding potency to rapidly induce broad multi-functional innate, humoral and cell-mediated immunity (CMI) responses that may result in life-long protection following a single vaccine dose in nearly all vaccinees^{7,8}. These favorable characteristics of the YF17D vaccine translate also to vectored vaccines based on the YF17D backbone⁹. YF17D is used as viral vector in two licensed human vaccines [Imojev® against Japanese encephalitis virus (JEV) and Dengvaxia® against dengue virus (DENV)]. For these two vaccines, genes encoding the YF17D surface antigens prM/E, have been swapped with those of JEV or DENV, respectively. Potent Zika virus vaccines based on this ChimeriVax approach are in preclinical development¹⁰.

YF17D is a small (+)-ss RNA live-attenuated virus with a limited vector capacity, but it has been shown to tolerate insertion of foreign antigens at two main sites in the viral polyprotein¹¹. Importantly, an insertion of foreign sequences is constrained by (i) the complex topology and post-translational processing of the YF17D polyprotein; and, (ii) the need to express the antigen of interest in an immunogenic, likely native, fold, to yield a potent recombinant vaccine.

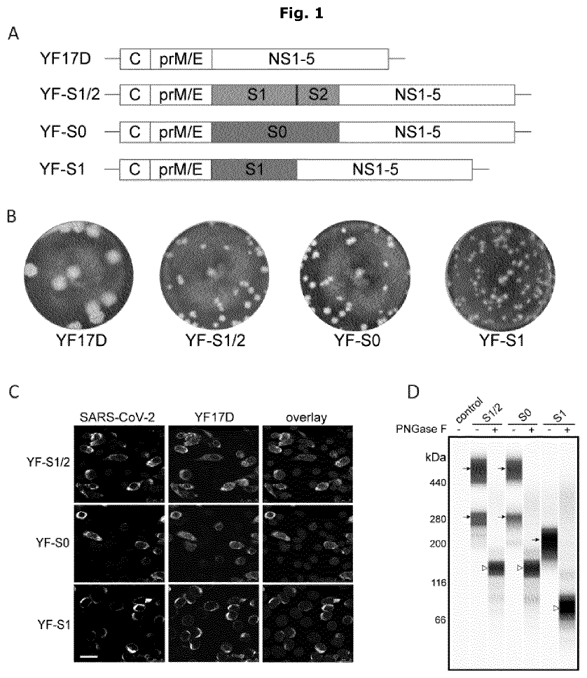

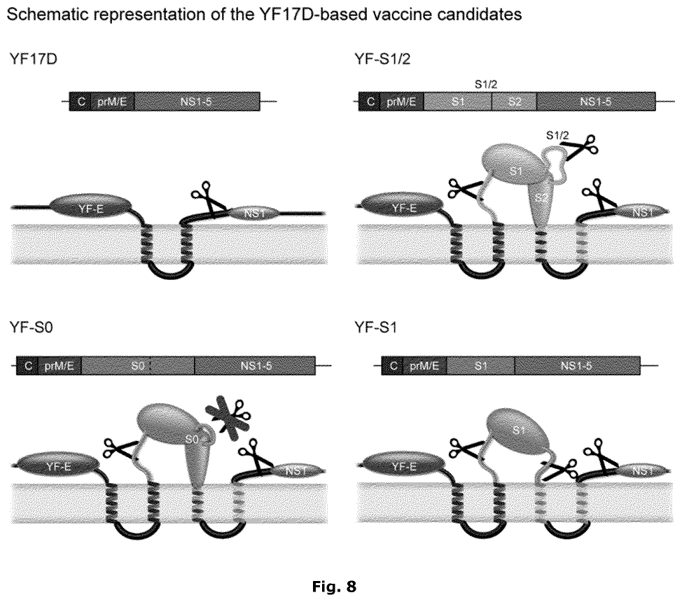

Using an advanced reverse genetics system for the generation of recombinant flaviviruses^{12,13}, a panel of YF17D-based COVID-19 vaccine candidates (YF-S) was designed. These express codon-optimized versions of the S protein [either in its native cleavable S1/2, or non-cleavable S0 version or its S1 subdomain] of the prototypic SARS-CoV-2 Wuhan-Hu-1 strain (GenBank: MN908947.3), as in-frame fusion within the YF17D-E/NS1 intergenic region (YF-S1/2, YF-S0 and YF-S1) (FIG. 1A, FIG. 8). As outlined below, variant YF-S0 was finally selected as lead vaccine candidate based on its superior immunogenicity, efficacy and favorable safety profile.

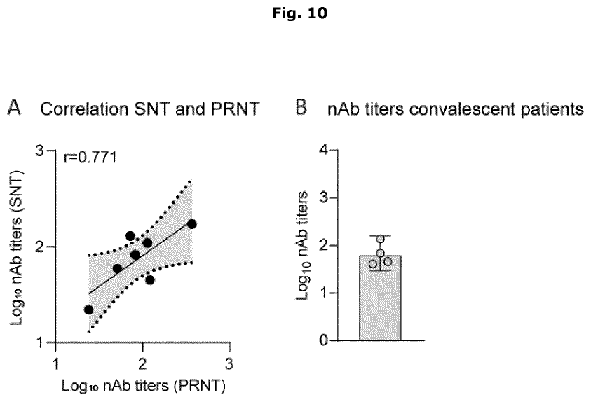

Infectious live-attenuated YF-S viruses were rescued by plasmid transfection into baby hamster kidney (BHK-21) cells, which are an established substrate for the production of biological agents and suitable for vaccine production at industrial scale when following the guidelines of the International Council for Harmonisation of Technical Requirements for Pharmaceuticals for Human Use (ICH), where the vaccine virus showed to be stable (FIG. 10). Transfected cells presented with a virus-induced cytopathic effect; infectious virus progeny was recovered from the supernatant and further characterized. Each construct results in a unique plaque phenotype, smaller than that of the parental YF17D (FIG. 1B), in line with some replicative trade-off posed by the inserted foreign sequences. S or S1 as well as YF17D antigens were readily visualized by double staining of YF-S infected cells with SARS-CoV-2 Spike and YF17D-specific antibodies (FIG. 1C). The expression of S or S1 by the panel of YF-S variants was confirmed by immunoblotting of lysates of freshly infected cells. Treatment with PNGase F allowed to demonstrate a proper glycosylation pattern (FIG. 1D). The full-length S1/2 and S0 antigens that contain the original S2 subunit (stalk and cytoplasmic domains) of S may be expected to (1) form spontaneously trimers10-12 and (2) to be intracellularly retained (reinforced by C-terminal fusion to an extra transmembrane domain known to function as endoplasmic reticulum retention signal).

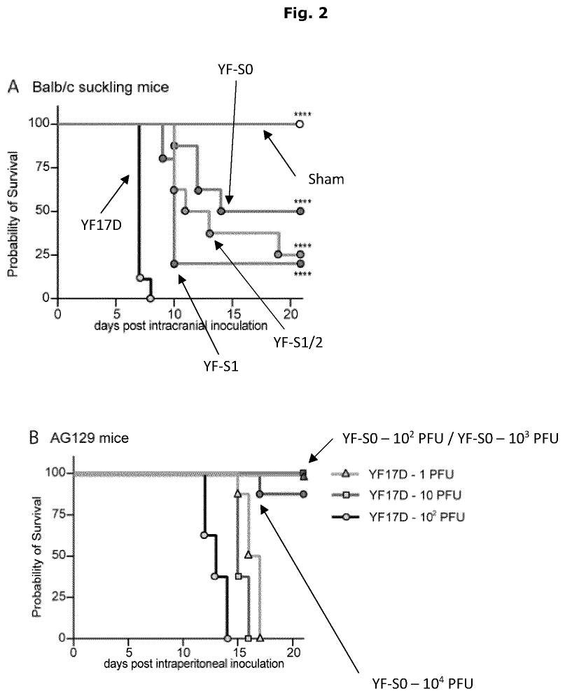

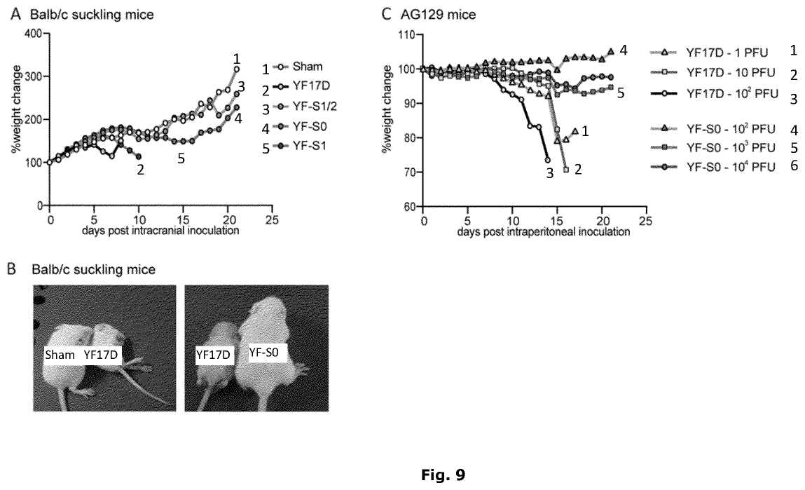

In line with a smaller plaque phenotype, intracranial (i.c.) inoculation of YF17D or the YF-S variants in suckling mice confirmed the attenuation of the different YF-S as compared to the empty vector YF17D (FIG. 2A and B and FIG. 9). Mouse pups inoculated i.c. with 100 plaque forming units (PFU) of the parental YF17D stopped growing (FIG. 9A) and succumbed to infection within seven days (median day of euthanasia; MDE) (FIG. 2A), whereas pups inoculated with the YF-S variants continued to grow. From the group inoculated with YF-S0 only half needed to be euthanized (MDE 17.5 days). For the YF-S1/2 and YF-S1 groups MDE was 12 and 10 days respectively; thus in particular YF-S0 has a markedly reduced neurovirulence. Likewise, YF-S0 is also highly attenuated in type I and II interferon receptor deficient AG129 mice, that are highly susceptible to (a neurotropic) YF17D infection^{13,14}. Whereas 1 PFU of YF17D resulted in neuro-invasion requiring euthanasia of all mice (MDE 16 days) (FIG. 2B), a 1000-fold higher inoculum of YF-S0 did not result in any disease (FIG. 9C) and only 1 in 12 animals that received a 10,000 higher inoculum needed to be euthanized (FIG. 2B). In summary, a set of transgenic replication-competent YF17D variants (YF-S) was generated that express different forms of the SARS-CoV-2 S antigen and that are highly attenuated in mice in terms of neurovirulence and neurotropism as compared to YF17D.

### 8.2 Immunogenicity and Protection Against SARS-CoV-2 Infection and COVID-19-Like Disease in a Stringent Hamster Model

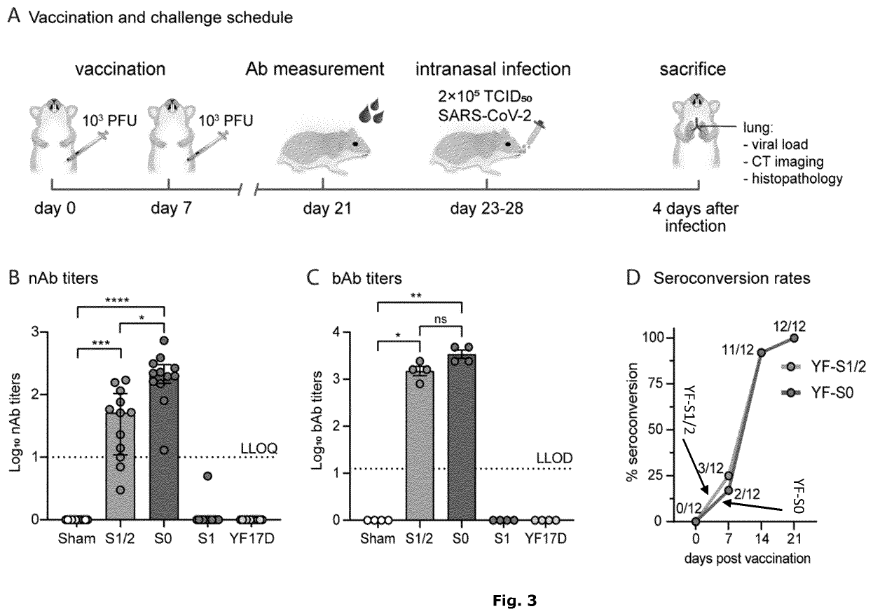

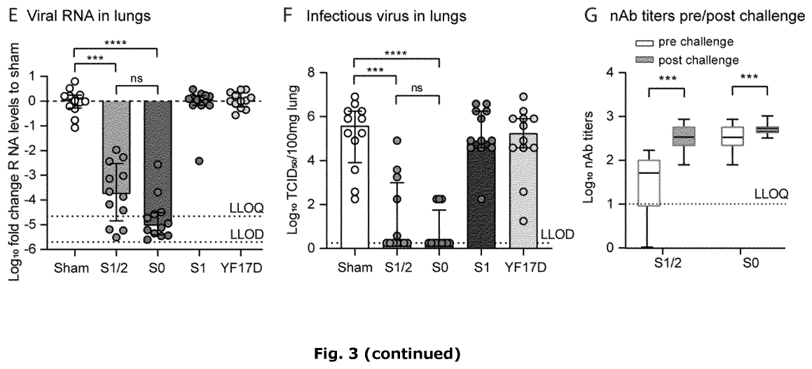

To assess the potency of the various vaccine constructs, a stringent hamster challenge model was developed. Animals were vaccinated at day 0 with 10³ PFU (i.p. route) of the different constructs or the negative controls and were boosted 7 days later with the same dose (FIG. 3A). At day 21 post-vaccination, all hamsters vaccinated with YF-S1/2 and YF-S0 (n=12 from two independent experiments) had seroconverted to high levels of S-specific IgG and virus nAbs (FIG. 3B,C; see FIG. 10 for benchmarking of SARS-CoV-2 serum neutralization test, SNT). For YF-S1/2 log₁₀ geometric mean titers (GMT) for IgG and nAbs were 3.2 (95% CI, 2.9-3.5) and 1.4 (95% CI, 1.1-1.9) respectively, while in the case of YF-S0 GMT values for IgG and nAbs of 3.5 (95% CI, 3.3-3.8) and 2.2 (95% CI, 1.9-2.6) were measured, with rapid seroconversion kinetics (50% seroconversion rate <2 weeks; FIG. 3D). By contrast, only 1 out of 12 hamsters that had received YF-S1 seroconverted and this with a low level of nAbs. This indicates the need for a full-length S antigen to elicit an adequate humoral immune response.

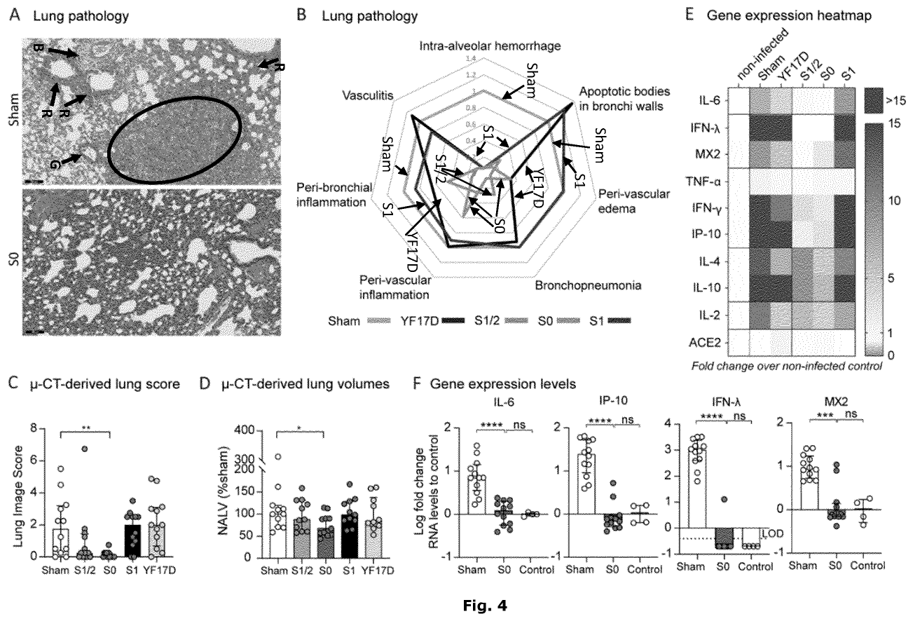

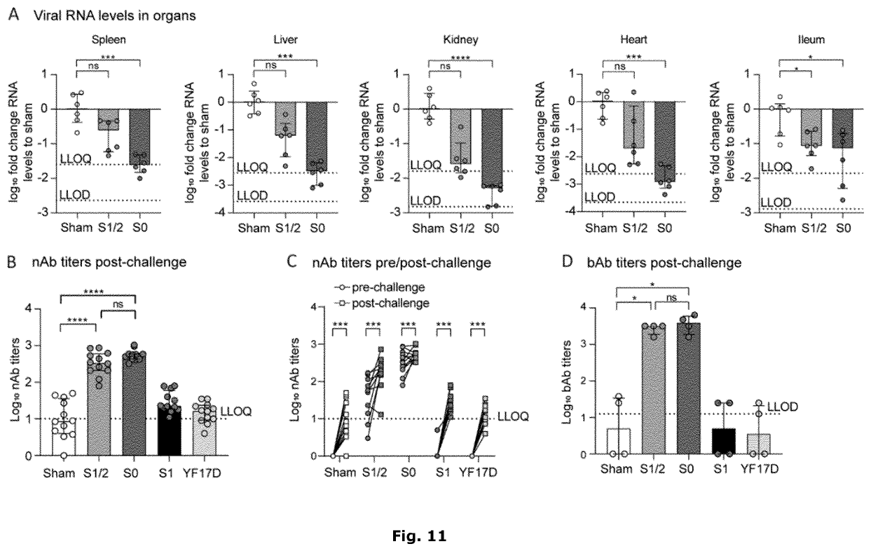

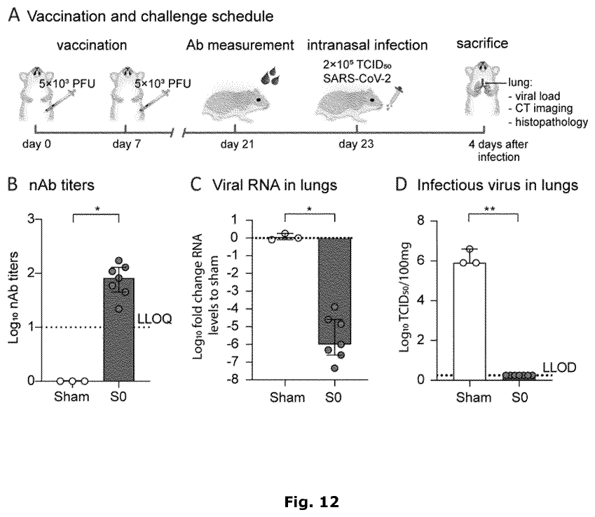

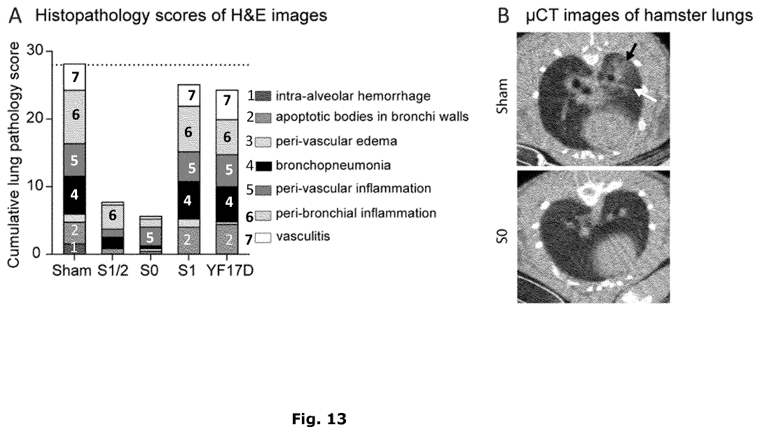

Next, vaccinated hamsters were challenged intranasally (either at day 23 or day 28 post vaccination) with 2×10⁵ PFU of SARS-CoV-2. At day 4 post-infection, high viral loads were detected in lungs of sham-vaccinated controls and animals vaccinated with YF17D as matched placebo (FIG. 4A, B). Infection was characterized by a severe lung pathology with multifocal necrotizing bronchiolitis, leukocyte infiltration and edema, resembling findings in patients with severe COVID-19 bronchopneumonia (FIG. 4A specimen pictures and 4B radar plot). By contrast, hamsters vaccinated with YF-S0 were protected against this aggressive challenge (FIG. 3E-F). As compared to sham-vaccinated controls, YF-S0 vaccinated animals had a median reduction of 5 log₁₀ (IQR, 4.5-5.4) in viral RNA loads (p<0.0001; FIG. 3D), and of 5.3 log₁₀ (IQR, 3.9-6.3) for infectious SARS-CoV-2 virus in the lungs (p<0.0001; FIG. 3E). Moreover, infectious virus was no longer detectable in 10 of 12 hamsters (two independent experiments), and viral RNA was reduced to non-quantifiable levels in their lungs. Residual RNA measured in 2 out of 12 animals may equally well represent residues of the high-titer inoculum as observed in non-human primate models¹⁵⁻¹⁸. Vaccination with YF-S0 (two doses of 10³ PFU) also efficiently prevented systemic viral dissemination; in most animals, no or only very low levels of viral RNA were detectable in spleen, liver, kidney and heart four days after infection (FIG. 11A). Similarly and in full support, a slightly different dose and schedule used for vaccination (5×10³ PFU of YF-S0 at day 0 and 7 respectively) resulted in all vaccinated hamsters (n=7) in respectively a 6 log₁₀ (IQR, 4.6-6.6) and 5.7 log₁₀ (IQR, 5.7-6.6) reduction of viral RNA and infectious virus titers as compared to sham (FIG. 12). Finally, vaccination with YF-S0 may induce saturating levels of nAbs thereby conferring sterilizing immunity, as demonstrated by the fact that in about half of the YF-S0 vaccinated hamsters no anamnestic antibody response was observed following challenge (FIGS. 3G and 11B-D (paired nAb analysis)). By contrast, in hamsters vaccinated with the second-best vaccine candidate YF-S1/2, nAb levels further increased following SARS-CoV-2 infection (in 11 out of 12 animals) whereby a plateau was only approached after challenge.

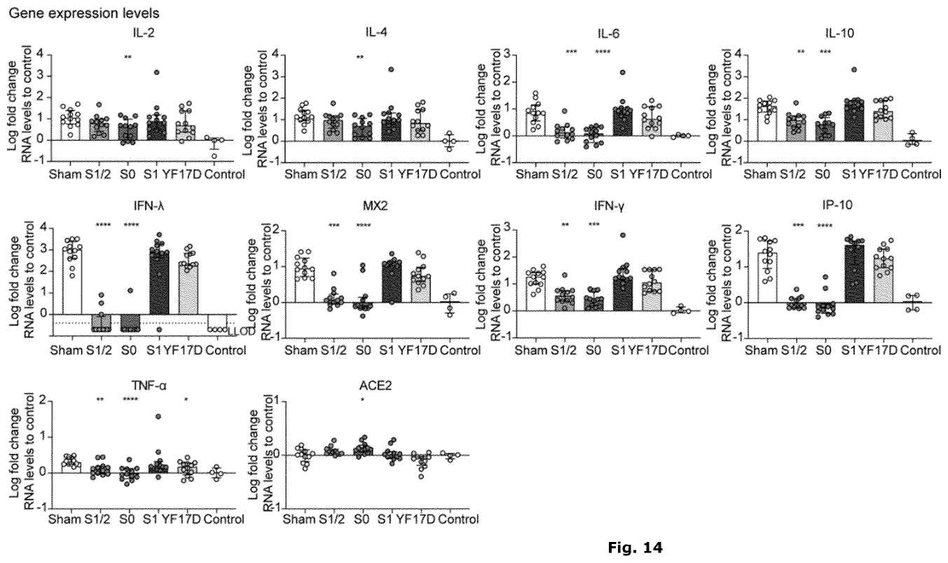

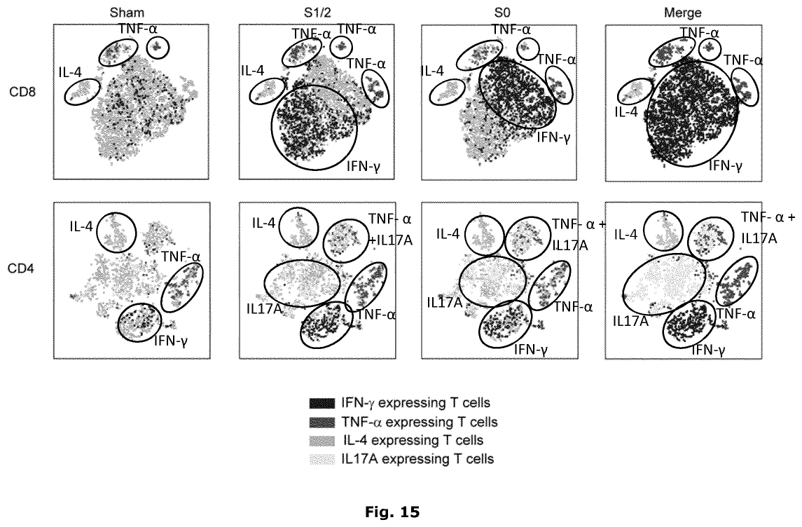

The lungs of YF-S0 vaccinated animals remained normal, or near to normal with no more signs of bronchopneumonia which is markedly different to sham-vaccinated animals (n=12 from two independent experiments; FIG. 4). The specific disease scores and biomarkers quantified included (i) a reduction or lack of detectable lung pathology as observed by histological inspection (FIG. 4A,B, FIG. 13A); and, (ii) a significant improvement of the individual lung scores (p=0.002) (FIG. 4C, FIG. 13B) and respiratory capacity (i.e* 32% less of lung volume obstructed; p=0.0323; FIG. 4D) in YF-S0 vaccinated animals as derived by micro-computed tomography (micro-CT) of the chest. In addition, immunization with YF-S0 resulted in an almost complete, in most cases full, normalization of the expression of cytokines, e.g., IL-6, IL-10, or IFN-γ in the lung, linked to disease exacerbation in COVID-19 (FIG. 4E,F and FIG. 14)¹⁹⁻²¹. Even the most sensitive markers of viral infection, such as the induction of antiviral Type III interferons (IFN-λ)²², or the expression of IFN-stimulated genes (ISG) such as MX2 and IP-10 in YF-S0 vaccinated animals showed no elevation as compared to levels in the lungs of untreated healthy controls (FIG. 4F and FIG. 14).

Overall, YF-S0 that expresses the non-cleavable S variant outcompeted construct YF-S1/2 expressing the cleavable version of S. This argues for the stabilized prefusion form of S serving as a relevant protective antigen for SARS-CoV-2. Moreover, in line with its failure to induce nAbs (FIG. 3B), construct YF-S1 expressing solely the hACE2 receptor-binding S1 domain (FIG. 1D) did not confer any protection against SARS-CoV-2 challenge in hamsters (FIG. 3E, F and FIG. 4).

### 8.3 Immunogenicity, in Particular a Favorable Th1 Polarization of Cell-Mediated Immunity in Mice

Since there are very few tools available to study CMI in hamsters, humoral and CMI responses elicited by the different YF-S constructs were studied in parallel in mice. Since YF17D does not readily replicate in wild-type mice^{23,24}, Ifnar^{-/-} mice that are deficient in Type I interferon signaling and that are hence susceptible to vaccination with YF17D, were employed^{10,24,25}.

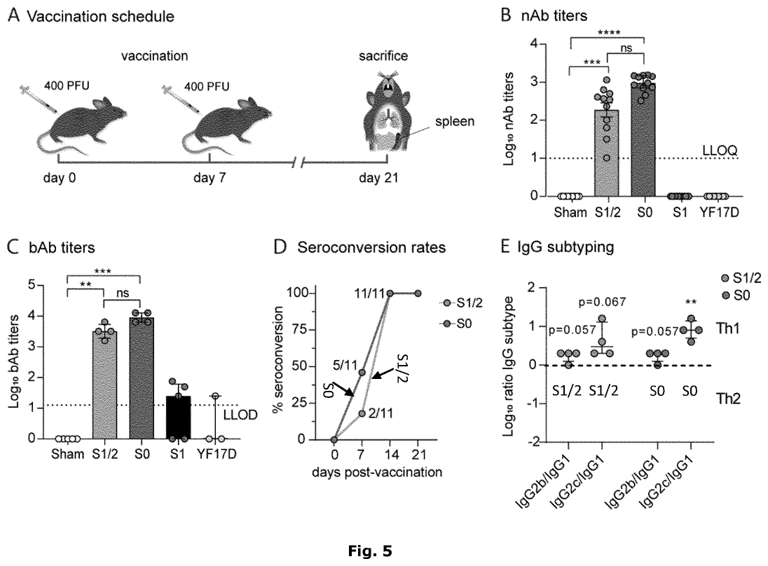

Mice were vaccinated with 400 PFU (of either of the YF-S variants, YF17D or sham) at day 0 and were boosted with the same dose 7 days later (FIG. 5A). At day 21 all YF-S1/2 and YF-S0 vaccinated mice (n>9 in three independent experiments) had seroconverted to high levels of S-specific IgG and nAbs with log₁₀ GMT of 3.5 (95% CI, 3.1-3.9) for IgG and 2.2 (95% CI, 1.7-2.7) for nAbs in the case of YF-S1/2, or 4.0 (95% CI, 3.7-4.2) for IgG and 3.0 (95% CI, 2.8-3.1) for nAbs in the case of YF-S0 (FIG. 5B,C). Importantly, seroconversion to S-specific IgG was detectable as early as 7 days after the first immunization (FIG. 5D). Isotyping of IgG revealed an excess of IgG2b/c over IgG1 indicating a dominant pro-inflammatory and hence antiviral (Th1) polarization of the immune response (FIG. 5E) which is considered important for vaccine-induced protection against SARS-CoV-2²⁶⁻²⁸. Alike in hamsters, YF-S1 failed to induce SARS-CoV-2 nAbs (FIG. 5B,C). However, high levels of YF nAbs were conjointly induced by all constructs confirming a consistent immunization (FIG. 15).

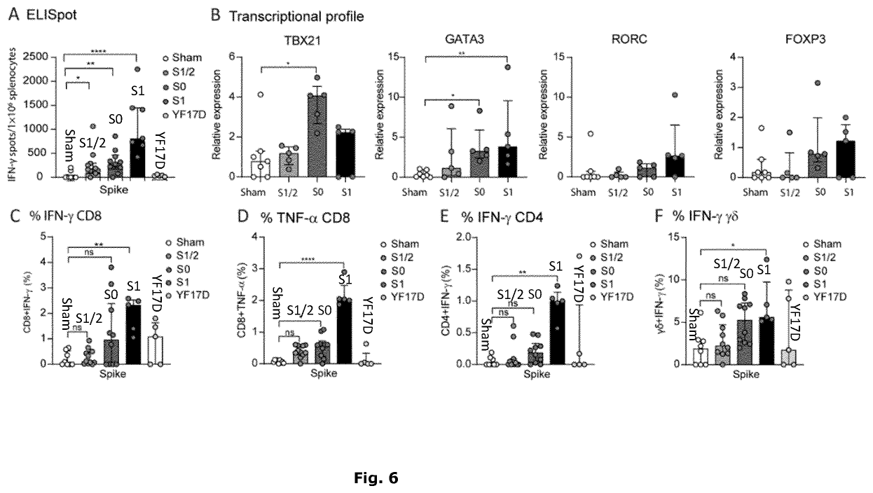

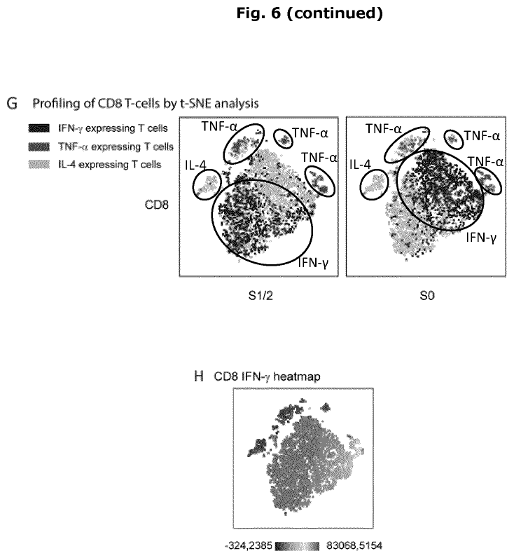

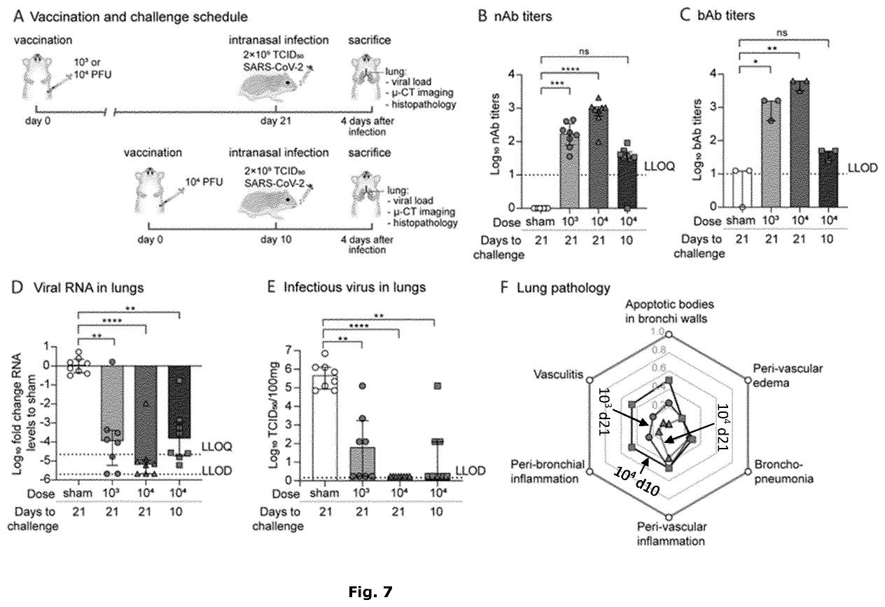

To assess SARS-CoV-2-specific CMI responses that play a pivotal role for the shaping and longevity of vaccine-induced immunity as well as in the pathogenesis of COVID-19^{29,30}, splenocytes from vaccinated mice were incubated with a tiled peptide library spanning the entire S protein as recall antigen. In general, vaccination with any of the YF-S variants resulted in marked S-specific T-cell responses with a favorable Th1-polarization as detected by IFN-γ ELISpot (FIG. 6A), further supported by an upregulation of T-bet (TBX21), in particular in cells isolated from YF-S0 vaccinated mice (p=0.0198, n=5). This CMI profile was balanced by a concomitant elevation of GATA-3 levels (GATA3; driving Th2; p=0.016), but no marked overexpression of RORγt (RORC; Th17) or FoxP3 (FOXP3; Treg) (FIG. 6B). Intriguingly, in stark contrast to its failure to induce nAbs in mice (FIG. 5A,C), or protection in hamsters (FIGS. 2 and 3), YF-S1 vaccinated animals had a greater number of S-specific splenocytes (p<0.0001, n=7) than those vaccinated with YF-S1/2 or YF-S0 (FIG. 6A). Thus, even a vigorous CMI may not be sufficient for vaccine efficacy. A more in-depth profiling of the T-cell compartment by means of intracellular cytokine staining (ICS) and flow cytometry confirmed the presence of S-specific IFN-γ and TNF-α expressing CD8⁺ T-lymphocytes, and of IFN-γ expressing CD4⁺ (FIG. 6E) and γ/δ T lymphocytes (FIG. 6F), in particular in YF-S0 immunized animals. A specific and pronounced elevation of other markers such as IL-4 (Th2 polarization), IL-17A (Th17), or FoxP3 (regulatory T-cells) was not observed for YF-S1/2 or YF-S0. This phenotype is supported by t-SNE plot analysis of the respective T-cell populations in YF-S1/2 and YF-S0 vaccinated mice (FIGS. 6G and 15 tSNE) showing an increased percentage of IFN-γ expressing cells. It further revealed, firstly, a similar composition of either CD4⁺ cell sets, comprising an equally balanced mixture of Th1 (IFN-γ⁺ and/or TNF-α⁺) and Th2 (IL-4⁺) cells, and possibly a slight raise in Th17 cells in the case of the YF-S0 vaccinated animals. Likewise, secondly, for both constructs the CD8⁺ T-lymphocyte population was dominated by IFN-γ or TNF-α expressing cells, in line with the matched transcriptional profiles (FIG. 6B). Of note, though similar in numbers, both vaccines YF-S0 and YF-S1/2 showed a distinguished (non-overlapping) profile regarding the respective CD8⁺ T lymphocyte populations expressing IFN-γ. In fact, YF-S0 tended to induce S-specific CD8⁺ T-cells with a stronger expression of IFN-γ (FIGS. 6G and 15). In summary, YF-S0 induces a vigorous and balanced CMI response in mice with a favorable Th1 polarization, dominated by SARS-CoV-2 specific CD8⁺ T-cells expressing high levels of IFN-γ when encountering the SARS-CoV-2 S antigen.

### 8.4 Protection and Short Time to Benefit after Single-Dose Vaccination

Finally, vaccination of hamsters using a single-dose of YF-S0 induced high levels of nAbs and bAbs (FIGS. 7B and 7C) in a dose- and time-dependent manner. Furthermore, it appears a single 10⁴ PFU dose of YF-S0 yielded higher levels of nAbs (log₁₀ GMT 2.8; 95% CI: 2.5-3.2) at 21 days post-vaccination compared to the antibody levels in a prime-boost vaccination with two doses of 10³ PFU (log₁₀ GMT 2.2; 95% CI: 1.9-2.6) (p=0.039, two tailed Mann-Whitney test) (FIG. 3B). Also, this single-dose regimen resulted in efficient and full protection against SARS-CoV-2 challenge, assessed by absence of infectious virus in the lungs in 8 out of 8 animals (FIG. 7E). It should be noted that viral RNA at quantifiable levels was present in only 1 out of 8 animals (FIG. 7D). In addition, protective immunity was mounted rapidly. Already 10 days after vaccination, 5 out of 8 animals receiving 10⁴ PFU of YF-S0 were protected against stringent infection challenge (FIGS. 7D and 7E). Notably, the persistence of Nabs and binding antibodies during long-term follow-up hints at a considerable longevity of immunity induced by this single-dose vaccination.

### 8.5 Discussion

Vaccines against SARS-CoV-2 need to be safe and result rapidly, ideally after one single dose, in long-lasting protective immunity. Different SARS-CoV-2 vaccine candidates are being developed, and several are vector-based. Present inventors report encouraging results of YF17D-vectored SARS-CoV-2 vaccine candidates. The post-fusion (S1/2), pre-fusion (S0) as well as the RBD S1 domain (S1) of the SARS-CoV-2 Spike protein were inserted in the YF17D backbone to yield the YF-S1/2, YF-S0 and YF-S1, respectively (FIG. 8). The YF-S0 vaccine candidate, in particular, resulted in a robust humoral immune response in both, mice and Syrian hamsters.

Since SARS-CoV-2 replicates massively in the lungs of infected Syrian hamsters and results in major lung pathology^{2,31-33} present inventors selected this model to assess the potency of these three vaccine candidates. YF-S0 resulted in efficient protection against stringent SARS-CoV-2 challenge, comparable, if not more vigorous, to other vaccine candidates in non-human primate models^{16,17,34}. In about 40% of the YF-S0 vaccinated animals no increase in nAb levels (>2×) following SARS-CoV-2 challenge was observed, suggestive for sterilizing immunity (no anamnestic response). In experiments in which animals were challenged three weeks after single 10⁴ PFU dose vaccination, no infectious virus was detected in the lungs. Considering the severity of the model, it is remarkable, that in several animals that were challenged with SARS-CoV-2 already 10 days after vaccination no infectious virus could be recovered from the lungs.

Reduction of viral replication mitigated lung pathology in infected animals with a concomitant normalization of biomarkers associated with infection and disease (FIGS. 4 and 13). Likewise, in lungs of vaccinated and subsequently challenged hamsters no elevation of cytokines, such as IL-6, was noted (FIG. 4F). The vaccination of macaques with a relatively low subcutaneous dose of YF-S0 led to rapid seroconversion to high NAb titres. It is tempting to speculate that this encouraging potency may translate into a simple one-shot dosing regimen for clinical use in humans.

Moreover, YF-S0 showed in two mice models a favorable safety profile as compared to the parental YF17D vector (FIG. 2A and B), and is well-tolerated in hamsters and nonhuman primates. This is of importance as YF17D vaccine is contra-indicated in elderly and persons with underlying medical conditions. These preliminary, though encouraging, data suggest that YF-S0 might also be safe in those persons most vulnerable to COVID-19.

In addition, cell-mediated immunity (CMI) studied in mice revealed that YF-S0, besides efficiently inducing high titers of nAbs, favors a Th1 response. Such a Th1 polarization is considered relevant in light of a disease enhancement supposedly linked to a skewed Th2 immune²⁹ or antibody-dependent enhancement (ADE)³⁵. ADE may occur when virus-specific antibodies promote virus infection via various Fcγ receptor-mediated mechanisms, as suggested for an inactivated RSV post-fusion vaccine candidate³⁶. A Th2 polarization may cause an induction and dysregulation of alternatively activated ‘wound-healing’ monocytes/macrophages^{26-28,37} resulting in an overshooting inflammatory response (cytokine storm) thus leading to acute lung injury (ALI). No indication of such a disease enhancement was observed in the models of present inventors.

In conclusion, YF-S0 confers vigorous protective immunity against SARS-CoV-2 infection. Remarkably, this immunity can be achieved within 10 days following a single dose vaccination. In light of the threat SARS-CoV-2 will remain endemic with spikes of re-infection, as a recurring plague, vaccines with this profile may be ideally suited for population-wide immunization programs.

### 8.6 Methods

### Cells and Viruses

BHK-21J (baby hamster kidney fibroblasts) cells³⁷ were maintained in Minimum Essential Medium (Gibco), Vero E6 (African green monkey kidney, ATCC CRL-1586) and HEK-293T (human embryonic kidney cells) cells were maintained in Dulbecco's Modified Eagle Medium (Gibco). All media were supplemented with 10% fetal bovine serum (Hyclone), 2 mM L-glutamine (Gibco), 1% sodium bicarbonate (Gibco). BSR-T7/5 (T7 RNA polymerase expressing BHK-21)³⁸ cells were kept in DMEM supplemented with 0.5 mg/ml geneticin (Gibco).

For all challenge experiments in hamsters, SARS-CoV-2 strain BetaCov/Belgium/GHB-03021/2020 (EPI ISL 407976|2020-02-03) was used from passage P4 grown on Vero E6 cells as described. YF17D (Stamaril®, Sanofi-Pasteur) was passaged twice in Vero E6 cells before use.

### Vaccine Design and Construction

Different vaccine constructs were generated using an infectious cDNA clone of YF17D (in an inducible BAC expression vector pShuttle-YF17D, patent number WO2014174078 A1)^{10,12,39}. A panel of several SARS-CoV-2 vaccine candidates was engineered by inserting a codon optimized sequence of either the SARS-CoV-2 Spike protein (S) (GenBank: MN908947.3) or variants thereof into the full-length genome of YF17D (GenBank: X03700) as translational in-frame fusion within the YF-E/NS1 intergenic region¹¹⁻⁴⁰ (FIG. 8). The variants generated contained (i) either the S protein sequence from amino acid (aa) 14-1273, expressing S in its post-fusion and/or prefusion conformation (YF-S1/2 and YF-S0, respectively), or (ii) its subunit-S1 (aa 14-722; YF-S1). To ensure a proper YF topology and correct expression of different S antigens in the YF backbone, transmembrane domains derived from WNV were inserted.

The SARS2-CoV-2 vaccine candidates were cloned by combining the S cDNA (obtained after PCR on overlapping synthetic cDNA fragments; IDT) by a NEB Builder Cloning kit (New England Biolabs) into the pShuttle-YF17D backbone. NEB Builder reaction mixtures were transformed into E. coli EPI300 cells (Lucigen) and successful integration of the S protein cDNA was confirmed by Sanger sequencing. Recombinant plasmids were purified by column chromatography (Nucleobond Maxi Kit, Machery-Nagel) after growth over night, followed by an additional amplification of the BAC vector for six hours by addition of 2 mM L-arabinose as described¹⁰.

Infectious vaccine viruses were generated from plasmid constructs by transfection into BHK-21J cells using standard protocols (TransIT-LT1, Mirus Bio). The supernatant was harvested four days post-transfection when most of the cells showed signs of CPE. Infectious virus titers (PFU/ml) were determined by a plaque assay on BHK-21J cells as previously described^{10,14}. The presence of inserted sequences in generated vaccine virus stocks was confirmed by RNA extraction (Direct-zol RNA kit, Zymo Research) followed by RT-PCR (qScript XLT, Quanta) and Sanger sequencing, and by immunoblotting of freshly infected cells (see infra).

### Analysis of Genetic Stability of YF-S0 Vaccine Virus

To test the genetic stability of YF-S0 vaccine virus, virus supernatants recovered from transfected BHK-21 cells (P0) were plaque purified once (P1) and serially passaged on BHK-21 cells (P3-P6). Furthermore, the genetic stability of 25 plaque isolates from a second round of plaque purification were analysed after amplification (P4*). For the comparison of two different cell substrates, YF-S0 virus supernatants harvested from transfected Vero or BHK-21 cells were passaged once on Vero or BHK-21 cells, respectively.

For all passages, fresh cells were infected for 1 hour with a 1:2 dilution of the virus supernatant from the respective previous passage. After infection the cells were washed twice with PBS. Supernatants of the infected cells were routinely harvested 72 or 96 hours post infection for BHK-21 and Vero, respectively. The presence of inserted sequences in generated passages was confirmed by RNA extraction and DNase I treatment (Direct-zol RNA kit, Zymo Research) followed by RT-PCR (qScript XLT, Quanta) and Sanger sequencing, and by immunoblotting of freshly infected cells (see infra).

### Immunofluorescent Staining

In vitro antigen expression of different vaccine candidates was verified by immunofluorescent staining as described previously by Kum et al. 2018. Briefly, BHK-21J cells were infected with 100 PFU of the different YF-S vaccine candidates. Infected cells were stained three days post-infection (3 dpi). For detection of YF antigens polyclonal mouse anti-YF17D antiserum was used. For detection of SARS-CoV-2 Spike antigen rabbit SARS-CoV Spike S1 antibody (40150-RP01, Sino Biological) and rabbit SARS-CoV Spike primary antibody (40150-T62-COV2, Sino Biological) was used. Secondary antibodies were goat anti-mouse Alexa Fluor-594 and goat anti-rabbit Alexa Fluor-594 (Life Technologies). Cells were counterstained with DAPI (Sigma). All confocal fluorescent images were acquired using the same settings on a Leica TCS SP5 confocal microscope, employing a HCX PL APO 63x (NA 1.2) water immersion objective.

### Immunoblot Analysis (Simple Western)

Infected BHK21-J cells were harvested and washed once with ice cold phosphate buffered saline, and lysed in radioimmunoprecipitation assay buffer (Thermo Fisher Scientific) containing 1× protease inhibitor and phosphatase inhibitor cocktail (Thermo Fisher Scientific). After centrifugation at 15,000 rpm at 4° C. for 10 minutes, protein concentrations in the cleared lysates were measured using BCA (Thermo Fisher Scientific). Immunoblot analysis was performed by a Simple Western size-based protein assay (Protein Simple) following manufactures instructions. Briefly, after loading of 400 ng of total protein onto each capillary, specific S protein levels were identified using specific primary antibodies (NB100-56578, Novus Biologicals and 40150-T62-CoV2, Sino Biological Inc.), and HRP conjugated secondary antibody (Protein Simple). Chemiluminescence signals were analyzed using Compass software (Protein Simple). To evaluate the removal of N-linked oligosaccharides from the glycoprotein, protein extracts were treated with PNGase F according to manufactures instructions (NEB).

### Animals

Wild-type Syrian hamsters (Mesocricetus auratus) and BALB/c mice and pups were purchased from Janvier Laboratories, Le Genest-Saint-Isle, France. Ifnar1^{-/-41} and AG129⁴² were bred in-house. Six- to ten-weeks-old Ifnar−/− mice, six- to eight-weeks old AG129 mice and six- to eight-weeks-old female wild-type hamsters were used throughout the study.

### Animal Experiments

Animals were housed in couples (hamsters) or per five (mice) in individually ventilated isolator cages (IsoCage N—Biocontainment System, Tecniplast) with access to food and water ad libitum, and cage enrichment (cotton and cardboard play tunnels for mice, wood block for hamsters). Housing conditions and experimental procedures were approved by the Ethical Committee of KU Leuven (license P015-2020), following Institutional Guidelines approved by the Federation of European Laboratory Animal Science Associations (FELASA). Animals were euthanized by 100 μl (mice) or 500 μl (hamsters) of intraperitoneally administered Dolethal (200 mg/ml sodium pentobarbital, Vétoquinol SA).

### Immunization and Infection of Hamsters

Hamsters were intraperitoneally (i.p) vaccinated with the indicated amount of PFUs of the different vaccine constructs using a prime and boost regimen (at day 0 and 7). As a control, two groups were vaccinated at day 0 and day 7 with either 10³ PFU of YF17D or with MEM medium containing 2% FBS (sham). All animals were bled at day 21 to analyze serum for binding and neutralizing antibodies against SARS-CoV-2. At the indicated time after vaccination and prior to challenge, hamsters were anesthetized by intraperitoneal injection of a xylazine (16 mg/kg, XYL-M®, V.M.D.), ketamine (40 mg/kg, Nimatek®, EuroVet) and atropine (0.2 mg/kg, Sterop®) solution. Each animal was inoculated intranasally by gently adding 50 μl droplets of virus stock containing 2×10⁵ TCID₅₀ of SARS-CoV-2 on both nostrils. Animals were monitored daily for signs of disease (lethargy, heavy breathing or ruffled fur). Four days after challenge, all animals were euthanized to collect end sera and lung tissue in RNA later, MEM or formalin for gene-expression profiling, virus titration or histopathological analysis, respectively.

### Immunization of Mice

Ifnar1^{-/-} mice were i.p. vaccinated with different vaccine constructs by using a prime and boost of each 4×10² PFU (at day 0 and 7). As a control, two groups were vaccinated (at day 0 and 7) with either YF17D or sham. All mice were bled weekly and serum was separated by centrifugation for indirect immunofluorescence assay (IIFA) and serum neutralization test (SNT). Three weeks post first-vaccination, mice were euthanized, spleens were harvested for ELISpot, transcription factor analysis by qPCR and intracellular cytokine staining (ICS).

### Immunization and Infection Challenge of Cynomolgus Macaques

All housing and animal procedures took place at the BPRC, upon positive advice by the independent ethics committee (DEC-BPRC), under project licence AVD5020020209404 issued by the Central Committee for Animal Experiments, and following approval of the detailed study protocol by the institutional animal welfare body. All animal handlings were performed within the Department of Animal Science according to Dutch law, regularly inspected by the responsible national authority (Nederlandse Voedsel-en Warenautoriteit, NVWA), and the animal welfare body. Macaques were pair-housed with a socially compatible cage-mate and randomly assigned to two groups. Six (n=6) cynomolgus macaques vaccinated subcutaneously in the inner upper limbs using a dose of 10⁵ PFU of YF-S0 at days 0 (prime) and 7 (boost). As a control, n=6 macaques were vaccinated twice with 10⁵ PFU of a matched placebo vaccine, consisting of recombinant YF17D with an irrelevant control antigen with no sequence homology to SARS-CoV-2 inserted in the same location (E/NS1 junction). A temperature monitor was implanted in the abdominal cavity of each macaque three weeks before the start of the study (Anapill DSI) providing continuous real-time measurement of body temperature and activity. Health was checked daily and macaques monitored for appetite, general behaviour and stool consistency. Blood was collected for regular assessment of whole blood counts and clinical chemistry with no changes out of normal ranges detected. On day 21 after vaccination, all macaques were challenged by a combined intranasal-intratracheal inoculation with nominally 1.5×10⁴ TCID50 of SARS-CoV-2 (as determined by back titration on Vero cells) in total volume 5 ml; split over the trachea (4 ml) and nares (0.25 ml each). The resulting virus RNA loads were quantified in throat swabs using RT-qPCR as described with a lower limit of detection of 200 RNA copies per ml. After a follow-up for 21 days, macaques were euthanized for histological analysis of their lungs.

### SARS-CoV-2 RT-qPCR

The presence of infectious SARS-CoV-2 particles in lung homogenates was quantified by qPCR². Briefly, for quantification of viral RNA levels and gene expression after challenge, RNA was extracted from homogenized organs using the NucleoSpin™ Kit Plus (Macherey-Nagel), following the manufacturer's instructions. Reactions were performed using the iTaq™ Universal Probes One-Step RT-qPCR kit (BioRad), with primers and probes (Integrated DNA Technologies) listed in Supplementary Table S1. The relative RNA fold change was calculated with the 2^{-ΔΔCq} method⁴³ using housekeeping gene β-actin for normalization.

### End-Point Virus Titrations

To quantify infectious SARS-CoV-2 particles, endpoint titrations were performed on confluent Vero E6 cells in 96-well plates. Lung tissues were homogenized using bead disruption (Precellys) in 250 μL minimal essential medium and centrifuged (10,000 rpm, 5 min, 4° C.) to pellet the cell debris. Viral titers were calculated by the Reed and Muench method⁴⁴ and expressed as 50% tissue culture infectious dose (TCID₅₀) per mg tissue.

### Histology

For histological examination, lung tissues were fixed overnight in 4% formaldehyde and embedded in paraffin. Tissue sections (5 μm) were stained with hematoxylin and eosin and analyzed blindly for lung damage by an expert pathologist.

### Micro-Computed Tomography (CP and Image Analysis

To monitor the development of lung pathology after SARS-CoV-2 challenge, hamsters were imaged using an X-cube micro-computed tomography (CT) scanner (Molecubes) as described before². Quantification of reconstructed micro-CT data were performed with DataViewer and CTan software (Bruker Belgium). A semi-quantitative scoring of micro-CT data was performed as primary outcome measure and imaging-derived biomarkers (non-aerated lung volume) as secondary measures, as previously described^{2,45-48}.

### Neurovirulence in Suckling Mice and Neurotropism in AG129 Mice

BALB/c mice pups and AG129 mice were respectively intracranially or i.p. inoculated with the indicated PFU amount of YF17D and YF-S vaccine constructs and monitored daily for morbidity and mortality for 21 days post inoculation.

### Detection of Total Binding IgG and IgG Isotyping by Indirect Immunofluorescent Assay (HFA)

To detect SARS-CoV-2 specific antibodies in hamster and mouse serum, an in-house developed indirect IFA (IIFA) was used. Using CRISPR/Cas9, a CMV-SARS-CoV-2-Spike-Flag-IRES-mCherry-P2A-BlastiR cassette was stably integrated into the ROSA26 safe harbor locus of HEK293T cells⁴⁹. To determine SARS-CoV-2 Spike binding antibody end titers, 1/2 serial serum dilutions were made in 96-well plates on HEK293T-Spike stable cells and HEK293T wt cells in parallel. Goat-anti-mouse IgG Alexa Fluor 488 (A11001, Life Technologies), goat-anti-mouse IgG1, IgG2b and IgG2c Alexa Fluor 488 (respectively 115-545-205, 115-545-207 and 115-545-208 from Jackson ImmunoResearch) were used as secondary antibody. After counterstaining with DAPI, fluorescence in the blue channel (excitation at 386 nm) and the green channel (excitation at 485 nm) was measured with a Cell Insight CX5 High Content Screening platform (Thermo Fischer Scientific). Specific SARS2-CoV-2 Spike staining is characterized by cytoplasmic (ER) enrichment in the green channel. To quantify this specific SARS-CoV-2 Spike staining the difference in cytoplasmic vs. nuclear signal for the HEK293T wt conditions was subtracted from the difference in cytoplasmic vs. nuclear signal for the HEK293T SARS-CoV-2 Spike conditions. All positive values were considered as specific SARS-CoV-2 staining. The IIFA end titer of a sample is defined as the highest dilution that scored positive this way. Because of the limited volume of serum, IIFA end titers for all conditions were determined on minipools of two to three samples.

### Pseudotyped Virus Seroneutralization Test (SNT)

SARS-CoV-2 VSV pseudotypes were generated as described previously⁵⁰⁻⁵². Briefly, HEK-293T cells were transfected with a pCAGGS-SARS-CoV-2_{Δ18}-Flag expression plasmid encoding SARS-CoV-2 Spike protein carrying a C-terminal 18 amino acids deletion^{53,54}. One day post-transfection, cells were infected with VSVΔG expressing a GFP (green fluorescent protein) reporter gene (MOI 2) for 2h. The medium was changed with medium containing anti-VSV-G antibody (IL mouse hybridoma supernatant from CRL-2700; ATCC) to neutralize any residual VSV-G virus input⁵⁵. 24 h later supernatant containing SARS-CoV-2 VSV pseudotypes was harvested.

To quantify SARS-CoV-2 nAbs, serial dilutions of serum samples were incubated for 1 hour at 37° C. with an equal volume of SARS-CoV-2 pseudotyped VSV particles and inoculated on Vero E6 cells for 18 hours. Neutralizing titers (SNT₅₀) for YFV were determined with an in-house developed fluorescence based assay using a mCherry tagged variant of YF17D virus^{10,39}. To that end, serum dilutions were incubated in 96-well plates with the YF17D-mCherry virus for 1h at 37° C. after which serum-virus complexes were transferred for 72 h to BHK-21J cells. The percentage of GFP or mCherry expressing cells was quantified on a Cell Insight CX5/7 High Content Screening platform (Thermo Fischer Scientific) and neutralization IC₅₀ values were determined by fitting the serum neutralization dilution curve that is normalized to a virus (100%) and cell control (0%) in Graphpad Prism (GraphPad Software, Inc.).

### SARS-CoV-2 Plaque Reduction Neutralization Test (PRNT)

Sera were serially diluted with an equal volume of 70 PFU of SARS-CoV-2 before incubation at 37° C. for 1 h. Serum-virus complexes were added to Vero E6 cell monolayers in 24-well plates and incubated at 37° C. for 1 h. Three days later, overlays were removed and stained with 0.5% crystal violet after fixation with 3.7% PFA. Neutralization titers (PRNT₅₀) of the test serum samples were defined as the reciprocal of the highest test serum dilution resulting in a plaque reduction of at least 50%.

### Antigens for T Cell Assays

PepMix™ Yellow Fever (NS4B) (JPT-PM-YF-NS4B) and subpool-1 (158 overlapping 15-mers) of PepMix™ SARS-CoV-2 spike (JPT-PM-WCPV-S-2) were used as recall antigens for ELISpot and ICS. Diluted Vero E6 cell lysate (50 μg/mL) and a combination of PMA (50 ng/mL) (Sigma-Aldrich) and Ionomycin (250 ng/mL) (Sigma-Aldrich) served as negative and positive control, respectively.

### Intracellular Cytokine Staining (ICS) and Flow Cytometry

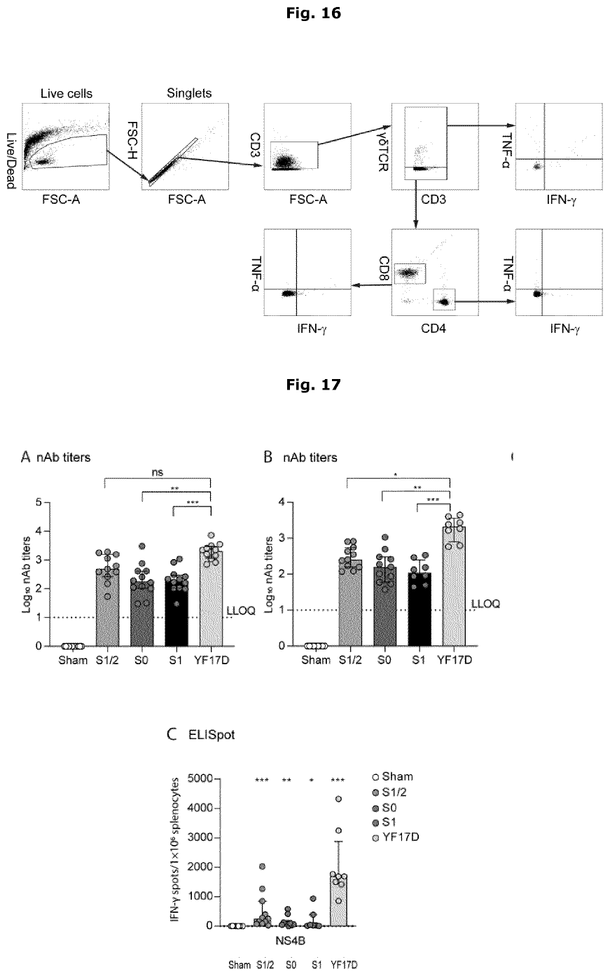

Fresh mouse splenocytes were incubated with 1.6 μg/mL Yellow Fever NS4B peptide; 1.6 μg/mL Spike peptide subpool-1; PMA (50 ng/mL)/Ionomycin (250 ng/mL) or 50 μg/mL Vero E6 cell for 18h at 37° C. After treatment with brefeldin A (Biolegend) for 4h, the splenocytes were stained for viability with Zombie Aqua™ Fixable Viability Kit (Biolegend) and Fc-receptors were blocked by the mouse FcR Blocking Reagent (Miltenyi Biotec) (0.5 μL/well) for 15 min in the dark at RT. Cells were then stained with extracellular markers BUV395 anti-CD3 (17A2) (BD), BV785 anti-CD4 (GK1.5) (Biolegend), APC/Cyanine7 anti-CD8 (53-6.7) (Biolegend) and PerCP/Cyanine5.5 anti-TCR γ/δ (GL3) (Biolegend) in Brilliant Stain Buffer (BD) before incubation on ice for 25 min. Cells were washed once with PBS and fixed/permeabilized for 30 min by using the FoxP3 transcription factor buffer kit (Thermo Fisher Scientific) according to the manufacturer's protocol. Finally, cells were intracellularly stained with following antibodies: PE anti-IL-4 (11B11), APC anti-IFN-γ (XMG1.2), PE/Dazzle™ 594 anti-TNF-α (MP6-XT22), Alexa Fluor® 488 anti-FOXP3 (MF-14), Brilliant Violet 421 anti-IL-17A (TC11-18H10.1) (all from Biolegend) and acquired on a BD LSRFortessa™ X-20 (BD). All measurements were calculated by subtracting from non-stimulated samples (incubated with non-infected Vero E6 cell lysates) from corresponding stimulated samples. The gating strategy employed for ICS analysis is depicted in FIG. 16. The strategy used for comparative expression profiling of vaccine-induced T-cell populations by t-distributed Stochastic Neighbor Embedding (t-SNE) analysis is outlined in Fig. S8. All flow cytometry data were analysed using FlowJo Version 10.6.2 (LLC)). t-SNE plot was generated in Flowjo after concatenating spike-specific CD4 and CD8 T cell separately based on gated splenocyte samples.

### ELISpot

ELISpot assays for the detection of IFN-γ-secreting mouse splenocytes were performed with mouse IFN-γ kit (ImmunoSpot® MIFNG-1M/5, CTL Europe GmbH). IFN-γ spots were visualized by stepwise addition of a biotinylated detection antibody, a streptavidin-enzyme conjugate and the substrate. Spots were counted using an ImmunoSpot® S6 Universal Reader (CTL Europe GmbH) and normalized by subtracting spots numbers from control samples (incubated with non-infected Vero E6 cell lysates) from the spot numbers of corresponding stimulated samples. Negative values were corrected to zero.

qPCR for Transcription Factor Profile

Spike peptide-stimulated splenocytes split were used for RNA extraction by using the sNucleoSpin™ Kit Plus kit (Macherey-Nagel). cDNA was generated by using a high-capacity cDNA Reverse Transcription Kit (Thermo Fisher Scientific). Real-time PCR was performed using the TaqMan gene expression assay (Applied Biosystems) on an ABI 7500 fast platform. Expression levels of TBX21, GATA3, RORC, FOXP3 (all from Integrated DNA Technologies) were normalized to the expression of GAPDH (IDT). Relative gene expression was assessed by using the 2^{-ΔΔCq} method.

### Statistical Analysis

GraphPad Prism (GraphPad Software, Inc.) was used for all statistical evaluations. The number of animals and independent experiments that were performed is indicated in the figure legends. Statistical significance was determined using the non-parametric Mann-Whitney U-test and Kruskal-Wallis test if not otherwise stated. Values were considered significantly different at P values of ≤0.05.

SUPPLEMENTARY TABLE S1
Primers and probes used for RT-qPCR

| Gene | Description | Oligonucleotide sequence |
|---|---|---|
| SARS-CoV-2 | Primer 1 | 5′-TTA CAA ACA TTG GCC GCA AA-3′ (SEQ |
| | | ID NO: 40) |
| | Primer 2 | 5′-GCG CGA CAT TCC GAA GAA-3′ (SEQ ID |
| | | NO: 41) |
| | Probe | 5′-FAM-ACA ATT TGC CCC CAG CGC TTC |
| | | AG-BHQ1-3′ (SEQ ID NO: 42) |
| Hamster | Primer 1 | 5′-GGG AAC TGT CAA AGG GTA CAG-3′ |
| ACE2 | | (SEQ ID NO: 43) |
| | Primer 2 | 5′-CCC TTC CTA CAT CAG TCC TAC T-3′ |
| | | (SEQ ID NO: 44) |
| | Probe | 5′-FAM-TCC CTG CTC ATT TGC TTG GTG |
| | | ACA-ZEN/IABkFQ-3′ (SEQ ID NO: 45) |
| Hamster | Primer 1 | 5′-GGC CAG GTC ATC ACC ATT-3′ (SEQ ID |
| ACTB | | NO: 46) |
| | Primer 2 | 5′-GAG TTG AAT GTA GTT TCG TGG ATG-3′ |
| | | (SEQ ID NO: 47) |
| | Probe | 5′-Cy5-TTT CCA GCC TTC CTT CCT GGG TAT |
| | | G-IBRQ-3′ (SEQ ID NO: 48) |
| Hamster | Primer 1 | 5′-TTT CTC CAT GCT GCT GTT GAA-3′ (SEQ |
| IFN-γ | | ID NO: 49) |
| | Primer 2 | 5′-GGC CAT CCA GAG GAG CAT AG-3′ (SEQ |
| | | ID NO: 50) |
| | Probe | 5′-FAM-CAC CAT CAA GGC AGA CCT GTT |
| | | TGC TAA CTT-ZEN/IABkFQ-3′ (SEQ ID NO: |
| | | 51) |
| Hamster | Primer 1 | 5′-CCC ACC AGA TGC AAA GGA TT-3′ (SEQ |
| IFNγ | | ID NO: 52) |
| | Primer 2 | 5′-CTT GAG CAG CCA CTC TTC TAT G-3′ |
| | | (SEQ ID NO: 53) |
| | Probe | 5′-FAM-ACA TAG CCC GGT TCA AGT CTC |
| | | TGC-ZEN/IABkFQ-3′ (SEQ ID NO: 54) |
| Hamster IL-2 | Primer 1 | 5′-AAG CTC CTG TAA GTC CAG CAG TAA C- |
| | | 3′ (SEQ ID NO: 55) |
| | Primer 2 | 5′-GTG CAC CCA CTT CAA GCT CTA A-3′ |
| | | (SEQ ID NO: 56) |
| | Probe | 5′-FAM-AGG AAA CCC AGC AGC ACC TCG |
| | | AGC-ZEN/IABkFQ-3′ (SEQ ID NO: 57) |
| Hamster IL-4 | Primer 1 | 5′-GGG TCA CCT CAT GTT GGA AAT AAA-3′ |
| | | (SEQ ID NO: 58) |
| | Primer 2 | 5′-CCA CGG AGA AAG ACC TCA TCT G-3′ |
| | | (SEQ ID NO: 59) |
| | Probe | 5′-FAM-CAG GGC TTC CCA GGT GCT TCG |
| | | CAA GT-ZEN/IABkFQ-3′ (SEQ ID NO: 60) |
| Hamster IL-6 | Primer 1 | 5′-GGT ATG CTA AGG CAC AGC ACA CT-3′ |
| | | (SEQ ID NO: 61) |
| | Primer 2 | 5′-CCT GAA AGC ACT TGA AGA ATT CC-3′ |
| | | (SEQ ID NO: 62) |
| | Probe | 5′-FAM-AGA AGT CAC CAT GAG GTC TAC |
| | | TCG GCA AAA-ZEN/IABkFQ-3′ (SEQ ID NO: |
| | | 63) |
| Hamster IL- | Primer 1 | 5′-TTC TGG CCC GTG GTT CTC T-3′ (SEQ ID |
| 10 | | NO: 64) |
| | Primer 2 | 5′-GTT GCC AAA CCT TAT CAG AAA TGA-3′ |
| | | (SEQ ID NO: 65) |
| | Probe | 5′-FAM-CAG TTT TAC CTG GTA GAA GTG |
| | | ATG CCC CAG G-ZEN/IABkFQ-3′ (SEQ ID NO: |
| | | 66) |
| Hamster IP- | Primer 1 | 5′-GCC ATT CAT CCA CAG TTG ACA-3′ (SEQ |
| 10 | | ID NO: 67) |
| | Primer 2 | 5′-CAT GGT GCT GAC AGT GGA GTC T-3′ |
| | | (SEQ ID NO: 68) |
| | Probe | 5′-FAM-CGT CCC GAG CCA GCC AAC GA- |
| | | ZEN/IABkFQ-3′ (SEQ ID NO: 69) |
| Hamster MX2 | Primer 1 | 5′-CCA GTA ATG TGG ACA TTG CC-3′ (SEQ |
| | | ID NO: 70) |
| | Primer 2 | 5′-CAT CAA CGA CCT TGT CTT CAG TA-3′ |
| | | (SEQ ID NO: 71) |
| | Probe | 5′-FAM-TGT CCA CCA GAT CAG GCT TGG |
| | | TCA-ZEN/IABkFQ-3′ (SEQ ID NO: 72) |
| Hamster | Primer 1 | 5′-AGC TGG TTG TCT TTG AGA GAC ATG-3′ |
| TNF-α | | (SEQ ID NO: 73) |
| | Primer 2 | 5′-GGA GTG GCT GAG CCA TCG T-3′ (SEQ ID |
| | | NO: 74) |
| | Probe | 5′-FAM-CCA ATG CCC TCC TGG CCA ACG- |
| | | ZEN/IABkFQ-3′ (SEQ ID NO: 75) |
| Mouse | Primer 1 | 5′-GTG GAG TCA TAC GGA ACA TGT AG-3′ |
| GAPDH | | (SEQ ID NO: 76) |
| | Primer 2 | 5′-AAT GGT GAA GGT CGG TGT G-3′ (SEQ ID |
| | | NO: 77) |
| | Probe | 5′-/56-FAM/TGC AAA TGG/ZEN/CAG CCC |
| | | TGG TG/3IABkFQ/-3′ (SEQ ID NO: 78) |
| Mouse Tbx21 | Primer 1 | 5′-CAA GAC CAC ATC CAC AAA CAT C-3′ |
| | | (SEQ ID NO: 79) |
| | Primer 2 | 5′-TTC AAC CAG CAC CAG ACA G-3′ (SEQ ID |
| | | NO: 80) |
| | Probe | 5′-/56-FAM/TCA CTA AGC/ZEN/AAG GAC |
| | | GGC GAA TGT/3IABkFQ/-3′ (SEQ ID NO: 81) |
| Mouse | Primer 1 | 5′-GTC CCC ATT AGC GTT CCT C-3′ (SEQ ID |
| GATA3 | | NO: 82) |
| | Primer 2 | 5′-CCT TAT CAA GCC CAA GCG AA-3′ (SEQ |
| | | ID NO: 83) |
| | Probe | 5′-/56-FAM/TGT CCC TGC/ZEN/TCT CCT TGC |
| | | TGC/3IABkFQ/-3′ (SEQ ID NO: 84) |
| Mouse RORC | Primer 1 | 5′-GAG GTG CTG GAA GAT CTG C-3′ (SEQ ID |
| | | NO: 85) |
| | Primer 2 | 5′-TCT GCA AGA CTC ATC GAC AAG-3′ (SEQ |
| | | ID NO: 86) |
| | Probe | 5′-/56-FAM/CTA GCC AAG/ZEN/CTG CCA |
| | | CCC AAA G/3IABkFQ/-3′ (SEQ ID NO: 87) |
| Mouse | Primer 1 | 5′-CTG TCT TCC AAG TCT CGT CTG-3′ (SEQ |
| FOXP3 | | ID NO: 88) |
| | Primer 2 | 5′-CTG GTC TCT GCA GGT TTA GTG-3′ (SEQ |
| | | ID NO: 89) |
| | Probe | 5′-/56-FAM/CTG TGC CTG/ZEN/GTA TAT |
| | | GCT CCC GG/3IABkFQ/-3′ (SEQ ID NO: 90) |

### 8.7 Funding

This project has received funding from the European Union's Horizon 2020 research and innovation program under grant agreements No 101003627 (SCORE project) and No 733176 (RABYD-VAX consortium), funding from Bill and Melinda Gates Foundation under grant agreement INV-00636, and was supported by the Research Foundation Flanders (FWO) under the Excellence of Science (EOS) program (VirEOS project 30981113), the FWO Hercules Foundation (Caps-It infrastructure), and the KU Leuven Rega Foundation. This project received funding from the Research Foundation—Flanders (FWO) under Project No G0G4820N and the KU Leuven/UZ Leuven Covid-19 Fund under the COVAX-PREC project. J.M. and X.Z. were supported by grants from the China Scholarship Council (CSC). C.C. was supported by the FWO (FWO 1001719N). G.V.V. acknowledges grant support from KU Leuven Internal Funds (C24/17/061) and K.D. grant support from KU Leuven Internal Funds (C3/19/057 Lab of Excellence). G.O. is supported by funding from KU Leuven (C16/17/010) and from FWO-Vlaanderen. We appreciate the in-kind contribution of UCB Pharma, Brussels.

### 8.8 References

1 https://www.who.int/who-documents-detail/draft-landscape-of-covid-19-candidate-vaccines.
2 Boudewijns, R. et al. STAT2 signaling as double-edged sword restricting viral dissemination but driving severe pneumonia in SARS-CoV-2 infected hamsters. bioRxiv, 2020.2004.2023.056838, doi:10.1101/2020.04.23.056838 (2020).
3 Wang, L. et al. Importance of Neutralizing Monoclonal Antibodies Targeting Multiple Antigenic Sites on the Middle East Respiratory Syndrome Coronavirus Spike Glycoprotein To Avoid Neutralization Escape. J Virol 92, doi:10.1128/jvi.02002-17 (2018).
4 Buchholz, U. J. et al. Contributions of the structural proteins of severe acute respiratory syndrome coronavirus to protective immunity. Proc Natl Acad Sci USA 101, 9804-9809, doi:10.1073/pnas.0403492101 (2004).
5 Cao, Y. et al. Potent Neutralizing Antibodies against SARS-CoV-2 Identified by High-Throughput Single-Cell Sequencing of Convalescent Patients' B Cells. Cell, doi:https://doi.org/10.1016/j.cell.2020.05.025 (2020).
6 Wrapp, D. et al. Structural Basis for Potent Neutralization of Betacoronaviruses by Single-Domain Camelid Antibodies. Cell 181, 1004-1015.e1015, doi:https://doi.org/10.1016/j.cell.2020.04.031 (2020).
7 Querec, T. D. et al. Systems biology approach predicts immunogenicity of the yellow fever vaccine in humans. Nat Immunol 10, 116-125, doi:10.1038/ni.1688 (2009).
8 Barrett, A. D. & Teuwen, D. E. Yellow fever vaccine—how does it work and why do rare cases of serious adverse events take place? Curr Opin Immunol 21, 308-313, doi:10.1016/j.coi.2009.05.018 (2009).
9 Draper, S. J. & Heeney, J. L. Viruses as vaccine vectors for infectious diseases and cancer. Nat Rev Microbiol 8, 62-73, doi:10.1038/nrmicro2240 (2010).
10 Kum, D. B. et al. A yellow fever—Zika chimeric virus vaccine candidate protects against Zika infection and congenital malformations in mice. npj Vaccines 3, 56, doi:10.1038/s41541-018-0092-2 (2018).
11 Bonaldo, M. C., Sequeira, P. C. & Galler, R. The yellow fever 17D virus as a platform for new live attenuated vaccines. Hum Vaccin Immunother 10, 1256-1265, doi:10.4161/hv.28117 (2014).
12 Dallmeier, K. & Neyts, J. Simple and inexpensive three-step rapid amplification of cDNA 5′ ends using 5′ phosphorylated primers. Anal Biochem 434, 1-3, doi:10.1016/j.ab.2012.10.031 (2013).
13 Kum, D. B. et al. Limited evolution of the yellow fever virus 17d in a mouse infection model. Emerg Microbes Infect 8, 1734-1746, doi:10.1080/22221751.2019.1694394 (2019).
14 Mishra, N. et al. A Chimeric Japanese Encephalitis Vaccine Protects against Lethal Yellow Fever Virus Infection without Inducing Neutralizing Antibodies. mBio 11, doi:10.1128/mBio.02494-19 (2020).
15 Rockx, B. et al. Comparative pathogenesis of COVID-19, MERS, and SARS in a nonhuman primate model. Science 368, 1012-1015, doi:10.1126/science.abb7314 (2020).
16 van Doremalen, N. et al. ChAdOx1 nCoV-19 vaccination prevents SARS-CoV-2 pneumonia in rhesus macaques. bioRxiv, 2020.2005.2013.093195, doi:10.1101/2020.05.13.093195 (2020).
17 Yu, J. et al. DNA vaccine protection against SARS-CoV-2 in rhesus macaques. Science, doi:10.1126/science.abc6284 (2020).
18 Shi, J. et al. Susceptibility of ferrets, cats, dogs, and other domesticated animals to SARS-coronavirus 2. Science 368, 1016-1020, doi:10.1126/science.abb7015 (2020).
19 Zhu, N. et al. A Novel Coronavirus from Patients with Pneumonia in China, 2019. New England Journal of Medicine 382, 727-733, doi:10. 1056/NEJMoa2001017 (2020).
20 Huang, C. et al. Clinical features of patients infected with 2019 novel coronavirus in Wuhan, China. The Lancet 395, 497-506, doi:10.1016/S0140-6736(20)30183-5 (2020).
21 Wang, D. et al. Clinical Characteristics of 138 Hospitalized Patients With 2019 Novel Coronavirus-Infected Pneumonia in Wuhan, China. JAMA 323, 1061-1069, doi:10.1001/jama.2020.1585 (2020).
22 Prokunina-Olsson, L. et al. COVID-19 and emerging viral infections: The case for interferon lambda. Journal of Experimental Medicine 217, doi:10.1084/jem.20200653 (2020).
23 Meier, K. C., Gardner, C. L., Khoretonenko, M. V., Klimstra, W. B. & Ryman, K. D. A Mouse Model for Studying Viscerotropic Disease Caused by Yellow Fever Virus Infection. PLOS Pathogens 5, e1000614, doi:10.1371/journal.ppat.1000614 (2009).
24 Erickson, A. K. & Pfeiffer, J. K. Dynamic Viral Dissemination in Mice Infected with Yellow Fever Virus Strain 17D. Journal of Virology 87, 12392-12397, doi:10.1128/jvi.02149-13 (2013).
25 Watson, A. M., Lam, L. K., Klimstra, W. B. & Ryman, K. D. The 17D-204 Vaccine Strain-Induced Protection against Virulent Yellow Fever Virus Is Mediated by Humoral Immunity and CD4+ but not CD8+ T Cells. PLoS Pathog 12, e1005786, doi:10.1371/journal.ppat.1005786 (2016).
26 Channappanavar, R. et al. Dysregulated Type I Interferon and Inflammatory Monocyte-Macrophage Responses Cause Lethal Pneumonia in SARS-CoV-Infected Mice. Cell Host Microbe 19, 181-193, doi:10.1016/j.chom.2016.01.007 (2016).
27 Channappanavar, R. & Perlman, S. Pathogenic human coronavirus infections: causes and consequences of cytokine storm and immunopathology. Semin Immunopathol 39, 529-539, doi:10.1007/s00281-017-0629-x (2017).
28 Page, C. et al. Induction of alternatively activated macrophages enhances pathogenesis during severe acute respiratory syndrome coronavirus infection. J Virol 86, 13334-13349, doi:10.1128/jvi.01689-12 (2012).
29 Grifoni, A. et al. Targets of T Cell Responses to SARS-CoV-2 Coronavirus in Humans with COVID-19 Disease and Unexposed Individuals. Cell 181, 1489-1501.e1415, doi:10.1016/j.cell.2020.05.015 (2020).
30 Ni, L. et al. Detection of SARS-CoV-2-Specific Humoral and Cellular Immunity in COVID-19 Convalescent Individuals. Immunity 52, 971-977.e973, doi:10.1016/j.immuni.2020.04.023 (2020).
31 Chan, J. F.-W. et al. Simulation of the clinical and pathological manifestations of Coronavirus Disease 2019 (COVID-19) in golden Syrian hamster model: implications for disease pathogenesis and transmissibility. Clinical Infectious Diseases, doi:10.1093/cid/ciaa325 (2020).
32 Sia, S. F. et al. Pathogenesis and transmission of SARS-CoV-2 in golden hamsters. Nature, doi:10.1038/s41586-020-2342-5 (2020).
33 Imai, M. et al. Syrian hamsters as a small animal model for SARS-CoV-2 infection and countermeasure development. Proceedings of the National Academy of Sciences, 202009799, doi:10.1073/pnas.2009799117 (2020).
34 Gao, Q. et al. Development of an inactivated vaccine candidate for SARS-CoV-2. Science 369, 77-81, doi:10.1126/science.abc1932 (2020).
35 Liu, L. et al. Anti-spike IgG causes severe acute lung injury by skewing macrophage responses during acute SARS-CoV infection. JCI Insight 4, doi:10.1172/jci.insight.123158 (2019).
36 Smatti, M. K., Al Thani, A. A. & Yassine, H. M. Viral-Induced Enhanced Disease Illness. Front Microbiol 9, 2991-2991, doi:10.3389/fmicb.2018.02991 (2018).
37 Lindenbach, B. & Rice, C. M. Trans-Complementation of yellow fever virus NS1 reveals a role in early RNA replication. Journal of virology 71, 9608-9617, doi:10.1128/JVI.71.12.9608-9617.1997 (1998).
38 Buchholz, U. J., Finke, S. & Conzelmann, K. K. Generation of bovine respiratory syncytial virus (BRSV) from cDNA: BRSV NS2 is not essential for virus replication in tissue culture, and the human RSV leader region acts as a functional BRSV genome promoter. J Virol 73, 251-259 (1999).
39 Sharma, S. et al. Small-molecule inhibitors of TBK1 serve as an adjuvant for a plasmid-launched live-attenuated yellow fever vaccine. Hum Vaccin Immunother, 1-8, doi:10.1080/21645515.2020.1765621 (2020).
40 Bredenbeek, P. J. et al. A recombinant Yellow Fever 17D vaccine expressing Lassa virus glycoproteins. Virology 345, 299-304, doi:10.1016/j.virol.2005.12.001 (2006).
41 Müller, U. et al. Functional role of type I and type II interferons in antiviral defense. Science 264, 1918-1921, doi:10.1126/science.8009221 (1994).
42 van den Broek, M. F., Müller, U., Huang, S., Zinkernagel, R. M. & Aguet, M. Immune defence in mice lacking type I and/or type II interferon receptors. Immunol Rev 148, 5-18, doi:10.1111/j.1600-065x.1995.tb00090.x (1995).
43 Livak, K. J. & Schmittgen, T. D. Analysis of relative gene expression data using real-time quantitative PCR and the 2(-Delta Delta C(T)) Method. Methods 25, 402-408, doi:10.1006/meth.2001.1262 (2001).
44 Reed, L. J. & Muench, H. A SIMPLE METHOD OF ESTIMATING FIFTY PERCENT ENDPOINT S12. American Journal of Epidemiology 27, 493-497, doi:10.1093/oxfordjournals.aje.a118408 (1938).
45 Vandeghinste, B. et al. Iterative CT Reconstruction Using Shearlet-Based Regularization. Nuclear Science, IEEE Transactions on 60, 121, doi:10.1117/12.911057 (2012).
46 Vande Velde, G. et al. Longitudinal micro-CT provides biomarkers of lung disease that can be used to assess the effect of therapy in preclinical mouse models, and reveal compensatory changes in lung volume. Dis Model Mech 9, 91-98, doi:10.1242/dmm.020321 (2016).
47 Berghen, N. et al. Radiosafe micro-computed tomography for longitudinal evaluation of murine disease models. Sci Rep 9, 17598, doi:10.1038/s41598-019-53876-x (2019).
48 Kaptein, S. J. et al. Antiviral treatment of SARS-CoV-2-infected hamsters reveals a weak effect of favipiravir and a complete lack of effect for hydroxychloroquine. bioRxiv, 2020.2006.2019.159053, doi:10.1101/2020.06.19.159053 (2020).
49 Geisinger, J. M., Turan, S., Hernandez, S., Spector, L. P. & Calos, M. P. In vivo blunt-end cloning through CRISPR/Cas9-facilitated non-homologous end-joining. Nucleic Acids Res 44, e76, doi:10.1093/nar/gkv1542 (2016).
50 Whitt, M. A. Generation of VSV pseudotypes using recombinant AG-VSV for studies on virus entry, identification of entry inhibitors, and immune responses to vaccines. J Virol Methods 169, 365-374, doi:10.1016/j.jviromet.2010.08.006 (2010).
51 Berger Rentsch, M. & Zimmer, G. A vesicular stomatitis virus replicon-based bioassay for the rapid and sensitive determination of multi-species type I interferon. PLoS One 6, e25858, doi:10.1371/journal.pone.0025858 (2011).
52 Hoffmann, M. et al. SARS-CoV-2 Cell Entry Depends on ACE2 and TMPRSS2 and Is Blocked by a Clinically Proven Protease Inhibitor. Cell 181, 271-280.e278, doi:10.1016/j.cell.2020.02.052 (2020).
53 Fukushi, S. et al. Vesicular stomatitis virus pseudotyped with severe acute respiratory syndrome coronavirus spike protein. Journal of General Virology 86, 2269-2274, doi:https://doi.org/10.1099/vir.0.80955-0 (2005).
54 Wang, C. et al. Publisher Correction: A human monoclonal antibody blocking SARS-CoV-2 infection. Nat Commun 11, 2511, doi:10.1038/s41467-020-16452-w (2020).
55 Kleine-Weber, H. et al. Mutations in the Spike Protein of Middle East Respiratory Syndrome Coronavirus Transmitted in Korea Increase Resistance to Antibody-Mediated Neutralization. J Virol 93, doi:10.1128/jvi.0.01381-18 (2019).

### Example 9: Further Data

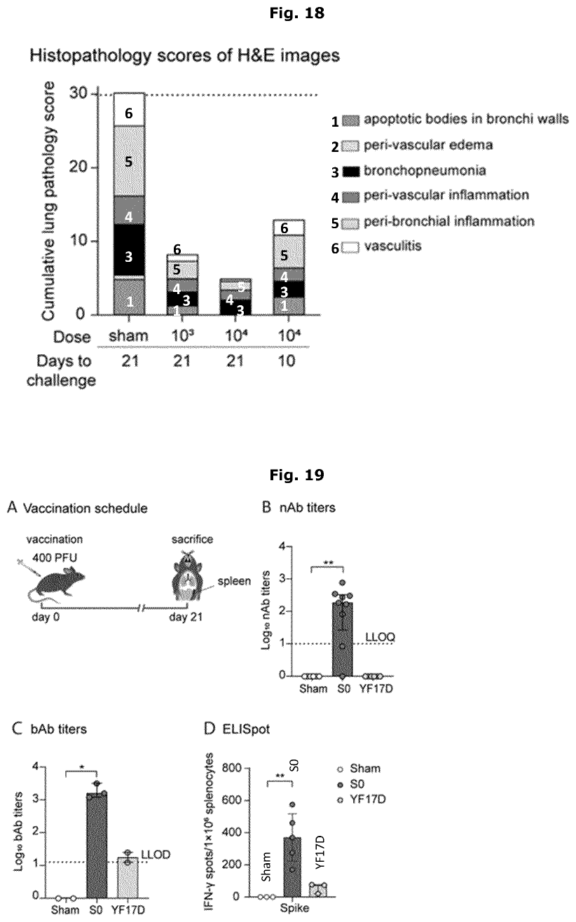

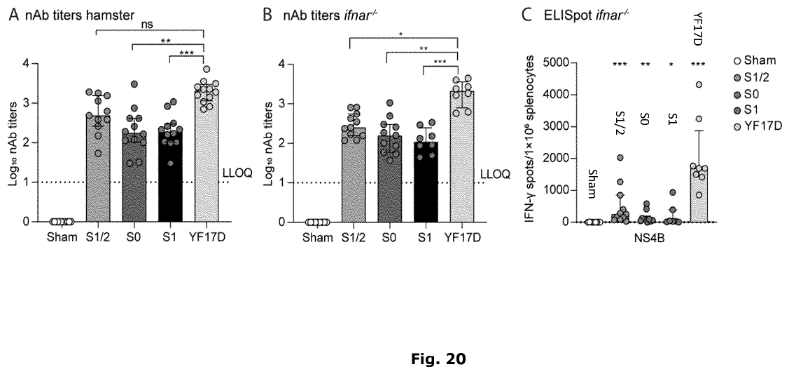

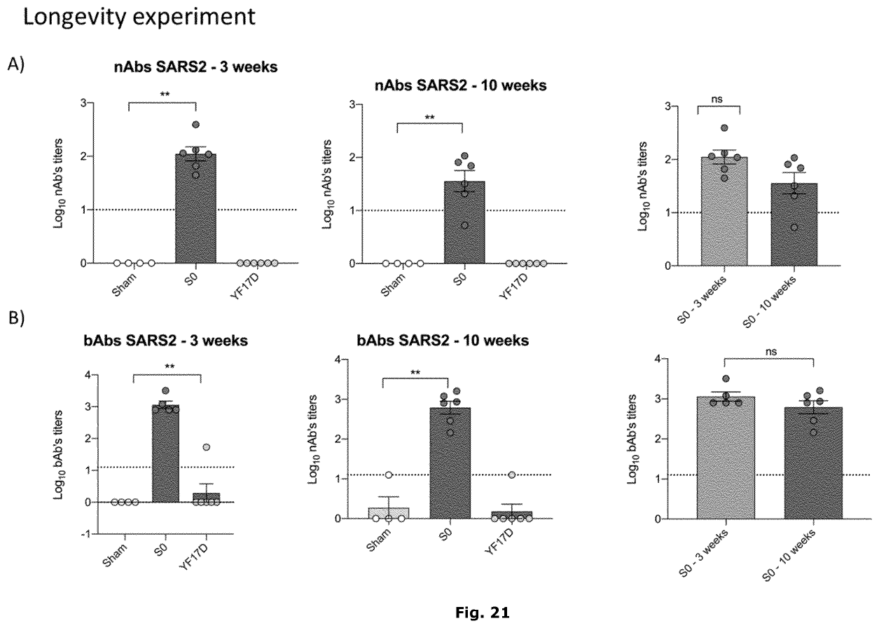

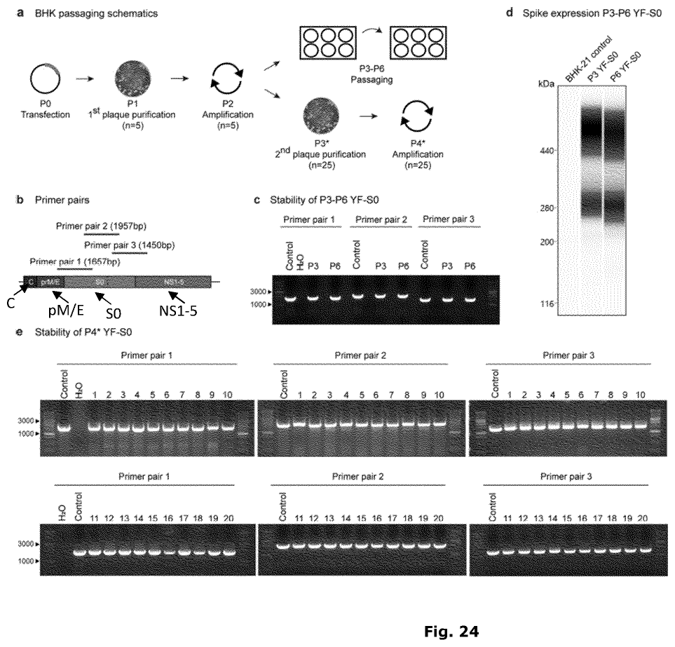

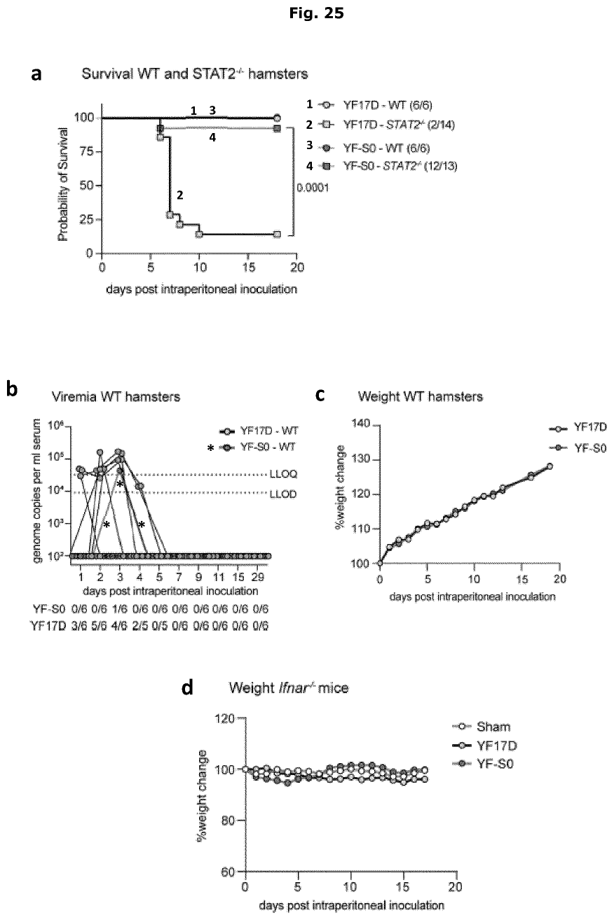

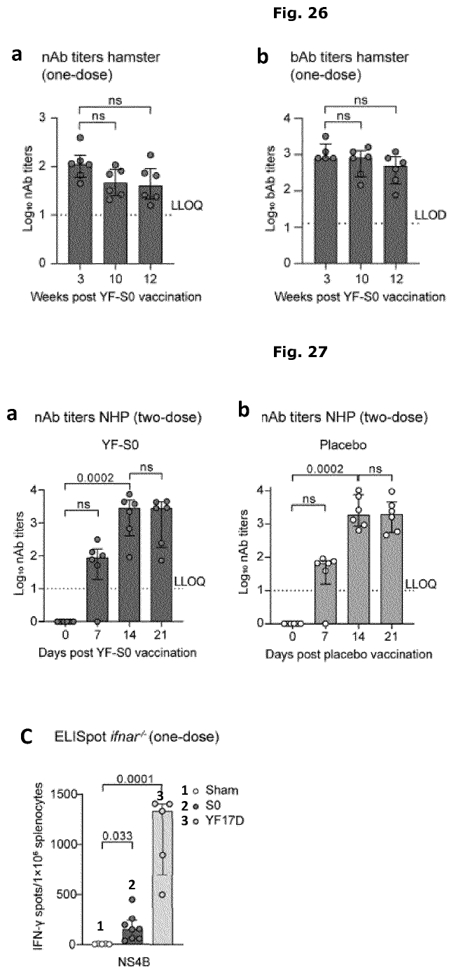

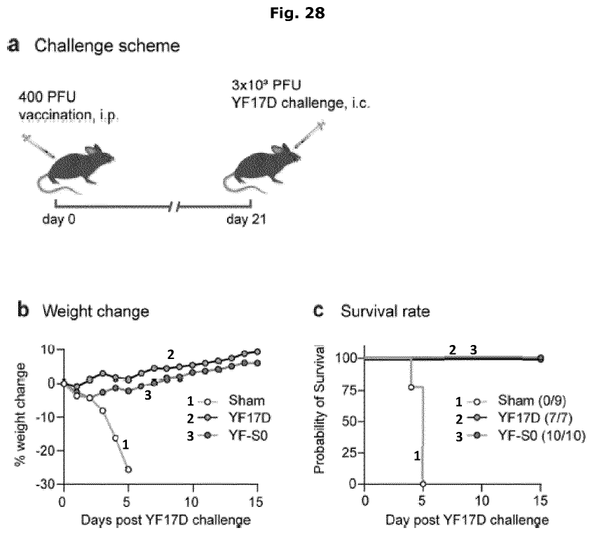

Additional results are illustrated in FIG. 17, FIG. 18, FIG. 19, FIG. 20, FIG. 21, FIG. 23, FIG. 24, FIG. 25, FIG. 26, FIG. 27 and FIG. 28.

FIG. 17. shows humoral immune response elicited by YF in hamsters and mice. FIG. 17 A-B show neutralizing antibodies (nAb) in hamsters (A) and ifnar^{-/-} mice (B) vaccinated with the different vaccine candidates (sera collected at day 21 post-vaccination in both experiments (two-dose vaccination schedule). FIG. 17 C shows the quantitative assessment YF17D specific cell-mediated immune response by ELISpot.

FIG. 18 shows lung pathology by histology. Cumulative histopathology score for signs of lung damage (vasculitis, peri-bronchial inflammation, peri-vascular inflammation, bronchopneumonia, peri-vascular edema, apoptotic bodies in bronchus walls) are indicated in H&E stained lung sections (dotted line—maximum score in sham-vaccinated group).

FIG. 19 shows that a humoral and cellular immune response is elicited by YF-S vaccine candidates in mice. FIG. 19A shows a schematic presentation of immunization and challenge schedule. Ifnar−/− mice were vaccinated once i.p. with 400 PFU YF-S0 (n=9), sham (white, n=6) or YF17D (grey, n=6). FIG. 19 B, C shows SARS-CoV-2 specific antibody levels at day 21 post-vaccination. FIG. 19 D shows the quantitative assessment of SARS-CoV-2 specific CMI response by ELISpot.

FIG. 20. Shows that YF17D-specific humoral immune response is elicited by YF-S in hamsters and mice. More particularly, FIG. 20A-B shows neutralizing antibodies (nAb) in hamsters (A) and ifnar−/− mice (B) vaccinated with the different vaccine candidates (sera collected at day 21 post-vaccination in both experiments (two-dose vaccination schedule)). FIG. 20 C shows the quantitative assessment of YF17D-specific cell-mediated immune response by ELISpot.

FIG. 21 shows the longevity of the humoral immune response following single vaccination in hamster. FIG. 21A shows neutralizing antibody (nAbs) titers and FIG. 21 B shows binding antibody titers (bAbs).

Six cynomolgus macaques were vaccinated with 10⁵ PFU of YF-S0 (similar to a human dose for YF17D or YF17D-based recombinant vaccines) via the subcutaneous route using the same schedule as in mice and hamsters. Six macaques received recombinant YF17D expressing an irrelevant control antigen as a matched placebo. No adverse signs or symptoms were observed. Macaques were bled weekly and assessed for seroconversion to NAb. At day 14 and day 21, all macaques vaccinated with YF-S0 had seroconverted to consistently high levels of virus Nabs, with geometric mean titres 2.6 (95% confidence interval of 2.4-2.8) and 2.5 (95% confidence interval of 2.3-2.7) respectively. These levels reach—if not exceed—those reported for other vaccine candidates (range of 0.3 to 2.6 log 10-transformed geometric mean titres), and correlate with protection as confirmed by a reduction in SARS-CoV-2 RNA levels in YF-S0-vaccinated macaques upon challenge. Seroconversion occurred rapidly: at day 7 (following a single dose) 2 out of 6 macaques receiving YF-S0 already had SARS-CoV-2 NAbs. In addition, YF-S0 induced protective levels of NAbs against yellow fever virus.

FIG. 23 shows immunogenicity and protective efficacy in cynomolgus macaques. Twelve cynomolgus macaques (M. fascicularis) were immunized twice (at day 0 and day 7) subcutaneously with 10⁵ PFU of YF-S0 (n=6) or matched placebo (n=6). On day 21 after vaccination, all macaques were challenged with 1.5×10⁴ TCID50 SARS-CoV-2. Histological examination of the lungs (day 21 after challenge) revealed no evidence of any SARS-CoV-2-induced pathology in macaques vaccinated with either YF-S0 or placebo.

FIG. 24 shows the genetic stability of YF-S0 during passaging in BHK-21 cells. YF-S0 vaccine virus recovered from transfected BHK-21 cells (P0) was plaque-purified once (P1) (n=5 plaque isolates), amplified (P2) and serially passaged on BHK-21 cells (P3-P6). In parallel, each amplified plaque isolate (P2) (n=5) from the first plaque purification was subjected to a second round of plaque purification (P3*) (n=25 plaque isolates) and amplification (P4*).

FIG. 25 shows the attenuation of YF-S vaccine candidates. FIG. 25 shows a survival curve of wild-type (WT) and STAT2-knockout (STAT2^{-/-}) hamsters inoculated intraperitoneally with 10⁴ PFU of YF17D or YF-S0. Wild-type hamsters inoculated with YF17D (n=6) and YF-S0 (n=6); STAT2^{-/-} hamsters inoculated with YF17D (n=14) and YF-S0 (n=13). FIG. 25 b, c show vaccine virus RNA (viraemia) in the serum (b) and weight evolution (c) of wild-type hamsters after intraperitoneal inoculation with 10⁴ PFU YF17D (n=6) or YF-S0 (n=6). The number of hamsters that showed viraemia on each day after inoculation is indicated (FIG. 25 b). FIG. 25 d shows the weight evolution of Ifnar^{-/-} mice after intraperitoneal inoculation with 400 PFU each at day 0 and 7 of YF-S0, YF17D and sham. Mice were inoculated with YF17D (n=5), YF-S0 (n=5) or sham (n=5).

FIG. 26. shows the imunogenicity and protective efficacy in hamsters after single dose vaccination Hamsters (n=6 per group from a single experiment) were vaccinated with a single dose of YF-S0 (10⁴ PFU intraperitoneally) and sera were collected at 3, 10 and 12 weeks after vaccination. NAbs (FIG. 26 a) and binding antibodies (FIG. 26 b) at the indicated weeks post vaccination.

FIG. 27. illustrates YF17D specific immune responses I macaques. FIG. 27 a, b show NAb titres after vaccination in macaques with YF-S0 (a) or placebo (b) (6 macaques per group from a single experiment); sera collected at indicated times after vaccination (two-dose vaccination schedule; FIG. 7). FIG. 27 c shows Ifnar^{-/-} mice vaccinated according to a single-dose vaccination schedule (YF-S0 (n=8), sham (n=5) and YF17D (n=5) from 2 independent experiments).

Spot counts were determined for IFNγ-secreting cells per 10⁶ splenocytes after stimulation with a YF17D NS4B peptide mixture.

FIG. 28 illustrates the protection from lethal YF17D. FIG. 28 a concerns Ifnar^{-/-} mice vaccinated with either a single 400 PFU intraperitoneal (i.p.) dose of YF17D (black) (n=7) or YF-S0 (n=10), or sham (grey, n=9). After 21 days, mice were challenged by intracranial (i.c.) inoculation with a uniformly lethal dose of 3×10³ PFU of YF17D and monitored for weight evolution (b) and survival (c).

1. A polynucleotide comprising a nucleotide sequence of a live, infectious, attenuated Flavivirus wherein a nucleotide sequence encoding the S1 and S2 subunit of a coronavirus Spike protein is located, so as to allow expression of a chimeric virus from said polynucleotide.

2. The polynucleotide according to claim 1, wherein the nucleotide sequence encoding the S1/S2 cleavage site is mutated, thereby preventing proteolytic processing of S protein in the S1 and S2 subunits.

3. The polynucleotide according to claim 1, wherein the nucleotide sequence encoding the S1 and S2 subunit of the coronavirus Spike protein is located 3′ of the nucleotide sequences encoding the envelope protein of the flavivirus and 5′ of the nucleotide sequences encoding the NS1 protein of the flavivirus.

4. The polynucleotide according to claim 1, wherein the nucleotide sequence encoding the S1 and S2 subunit of the coronavirus Spike protein does not comprise the nucleotide sequence encoding the signal peptide or part of the signal peptide of the coronavirus Spike protein, preferably wherein the nucleotide sequence encoding at least the S2 subunit of a coronavirus Spike protein does not comprise the first 39 nucleotides of the nucleotide sequence encoding the signal peptide of the coronavirus Spike protein.

5. The polynucleotide according to claim 3, wherein a nucleotide sequence encoding a transmembrane (TM) domain of a further flavivirus is located 3′ of the nucleotide sequence encoding the S1 and S2 subunit of the coronavirus Spike protein, and 5′ of the NS1 region of the NS1-NS5 region, preferably wherein the TM domain of a further flavivirus is a West Nile virus transmembrane domain 2 (WNV-TM2).

6. The polynucleotide according to claim 5, comprising 5′ to the nucleotide sequence encoding the S1 and S2 subunit of the coronavirus Spike protein, a sequence encoding an NS1 signal peptide.

7. The polynucleotide according to claim 1, wherein the nucleotide sequence encoding the S2′ cleavage site is mutated, thereby preventing proteolytic processing of the S2 unit.

8. The polynucleotide according to claim 1, wherein the coronavirus is severe acute respiratory syndrome coronavirus-2 (SARS-CoV-2).

9. The polynucleotide according to claim 1, wherein the Flavivirus is yellow fever virus.

10. The polynucleotide according to claim 1, wherein the Flavivirus is yellow fever 17 D (YF17D) virus.

11. The polynucleotide according to claim 1, comprising a sequence selected from the group consisting of SEQ ID NO: 3, SEQ ID NO: 5, and SEQ ID NO: 7, preferably comprising a sequence as defined by SEQ ID NO: 5.

12. The polynucleotide according to claim 1, which is a bacterial artificial chromosome (BAC).

13. A chimeric live, infectious, attenuated Flavivirus encoded by a polynucleotide according to claim 1.

14. A pharmaceutical composition comprising the polynucleotide according to claim 1, further comprising a pharmaceutically acceptable carrier, preferably wherein the pharmaceutical composition is a vaccine.

15. A polynucleotide according to claim 1, a chimeric virus, or a pharmaceutical composition for use as a medicament, wherein the medicament is a vaccine.

16. A polynucleotide according to claim 1, a chimeric virus, or a pharmaceutical composition for use in preventing a coronavirus infection, wherein the coronavirus infection is a SARS-CoV-2 infection.

17. An in vitro method of preparing a vaccine against a coronavirus infection, comprising the steps of:
a) providing a BAC which comprises:
an inducible bacterial ori sequence for amplification of said BAC to more than 10 copies per bacterial cell, and
a viral expression cassette comprising a cDNA of a chimeric virus comprising a polynucleotide according to claim 1, and comprising cis-regulatory elements for transcription of said viral cDNA in mammalian cells and for processing of the transcribed RNA into infectious RNA virus,
b) transfecting mammalian cells with the BAC of step a) and passaging the infected cells,
c) validating replicated virus of the transfected cells of step b) for virulence and the capacity of generating antibodies and inducing protection against coronavirus infection, and
d) cloning the virus validated in step c) into a vector, and
formulating the vector into a vaccine formulation.

18. The method according to claim 17, wherein the vector is BAC, which comprises an inducible bacterial ori sequence for amplification of said BAC to more than 10 copies per bacterial cell.

19. A pharmaceutical composition comprising the chimeric virus according to claim 13, further comprising a pharmaceutically acceptable carrier, wherein the pharmaceutical composition is a vaccine.


SEQUENCE LISTING (103 sequences)

SEQ ID NO: 1 - DNA, 3819 bp, SARS-CoV-2
atgtttgttttcttggtcctccttccactggtatcctcacaatgtgtgaatcttaccacccgaacccagcttcctcccgcctacacgaattcattcacgcggggtgtttattacccggataaagttttccggtccagtgtcctgcattcaacccaagacctctttctgccatttttttctaacgtgacgtggttccatgctatccatgtaagcggaaccaacggaaccaaacggttcgataatccggttctcccattcaacgatggggtttacttcgcatctacagaaaaatctaacataattagaggatggatttttgggactacgcttgacagtaagacccaatcactcttgatcgtgaacaatgcaaccaatgtagtaattaaggtttgcgagtttcaattttgtaatgatccatttttgggggtttattaccacaaaaacaataaatcctggatggaatccgaattcagagtgtatagcagcgctaacaattgcacatttgagtacgtgtcacaaccttttcttatggatcttgagggcaagcaagggaacttcaaaaatttgagggagttcgttttcaagaacatcgacggatactttaagatctattctaaacacacccccattaacttggtgcgagatttgcctcaaggcttctctgcacttgaaccgttggtggatcttcccattggcattaatattactcggttccagactttgttggcactgcatcgctcctatctcacgcccggagacagttcatctggatggactgcgggggctgccgcgtattacgtgggatacctgcagccgcgcacatttcttcttaaatacaacgagaacgggaccatcacagatgcagtggattgcgctcttgaccccctctccgaaacaaaatgtacgctcaagtctttcactgtagagaaagggatttatcagacatccaatttccgagtccagccaacagagagtatagtgcggttccctaacatcacaaatctttgtccgttcggggaagttttcaacgctacacgcttcgcaagtgtatacgcttggaatagaaagaggatctctaattgtgtggcagattactctgtgctctacaattccgcatctttctcaaccttcaagtgttacggagtttcacctacgaagctgaacgacctttgctttactaatgtatatgcagatagttttgtcatcaggggcgatgaagttcgacagatagcgcccggccagacaggaaagatcgcggactacaattataaactccctgatgatttcaccgggtgcgtgatcgcgtggaactctaataacctcgactccaaagtaggcggtaactacaattacttgtatcgattgtttagaaaatcaaaccttaaaccgttcgagcgggatatctctaccgagatataccaagcaggctctacaccgtgcaatggagtcgaaggttttaactgctacttccccttgcaatcatacgggtttcagcctaccaacggtgtaggataccagccttacagggttgtggtactttcatttgagctcctgcacgctcccgcaactgtctgtgggcccaaaaagagcactaaccttgttaaaaataaatgcgtcaactttaacttcaatggcctcactggcaccggcgtgctcactgaaagcaataagaaattccttccttttcagcagtttgggcgagacatagcggataccacggacgcagtacgggatcctcaaacccttgaaatccttgacataacgccttgctcttttgggggagtaagcgtaatcacgcctggaaccaacacctccaatcaggttgctgtgctgtaccaggatgtaaactgcaccgaggtaccggtagccattcacgcggatcagctgactcccacatggcgagtgtattctacaggtagtaatgtgtttcagacccgagcagggtgtttgataggggcggagcacgtcaacaactcatacgagtgcgatatacccattggggctggtatatgtgcatcctaccagacgcagacgaactctcctcgccgcgctcggtctgttgcatctcaatcaattattgcatacactatgtcactgggggctgagaattcagtagcctactctaacaacagcatcgcgattcccactaacttcacaattagtgtgactaccgagatcctgccagtatccatgactaaaactagcgtagattgtactatgtacatctgcggcgattcaactgagtgttcaaacctcctcttgcaatacgggtcattttgtacccaattgaatcgagctctgaccggcatcgcggtcgaacaggacaaaaatactcaagaggtatttgcccaggtgaagcagatttacaaaacaccccctatcaaggatttcgggggcttcaacttcagccagatactgccagacccctcaaaaccgagcaagcgctccttcatagaagatcttcttttcaacaaagttaccctcgcggatgctggtttcattaaacagtatggggactgtctcggcgacattgctgctagagacctcatctgcgcgcaaaagttcaatggacttacggtcctgccccctctcctcactgatgaaatgattgctcaatatacgtccgcgttgttggcgggaactataaccagtgggtggacgttcggcgctggcgccgcgcttcaaatcccatttgcgatgcaaatggcgtatcgcttcaacggcatcggagtaactcaaaacgttctgtacgaaaatcaaaaactcattgcgaaccagtttaattcagcgatcggtaaaatccaggacagcctgagctccacagcgagtgcactcgggaagctccaggatgtggtaaatcagaacgctcaagcgttgaacacactcgtcaagcagctgtcaagtaactttggcgcgatttcatctgtattgaatgacattctctctcgccttgataaggtggaagccgaagtccagattgatcgcctgattactgggcggcttcagtccctccaaacatacgtcactcagcaacttattagagccgccgaaattagggcaagtgcgaatctggccgcgacaaaaatgtctgaatgtgtgctggggcagagcaagagagtcgatttttgcggtaaggggtatcaccttatgtcttttcctcagtctgcccctcacggagtagtgtttctccacgttacgtatgtcccagcccaagagaaaaactttaccactgcgccggctatttgtcatgacggtaaagcacactttccacgcgaaggtgtgttcgtctccaacggcacccactggtttgtaacgcagaggaacttctacgagcctcagataattaccacggacaacacgttcgtctcaggtaactgcgacgtcgtaattggtattgtaaataacaccgtgtacgacccgctccagccggagctggactccttcaaagaggagcttgacaagtattttaagaatcacacttcaccggatgtagacctgggggatatttccggcataaacgcttccgtggttaacatacagaaagagatagatcgactgaacgaggtagcgaaaaacttgaatgagtctttgatagacctgcaagaattgggaaaatatgaacaatatattaagtggccctggtatatttggcttggtttcatagccggtttgattgccatcgtcatggtaactataatgctttgttgcatgacaagttgctgctcttgcctcaaagggtgctgctcctgtggaagttgttgcaagttcgatgaggatgattctgagccagtgcttaagggtgtcaaactgcattatacg

SEQ ID NO: 2 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - DNA, 4182 bp, Artificial Sequence
  construct 1
aaggtcatcatgggggcggtacttatatgggttggcatcaacacaagaaacatgacaatgtccatgagcatgatcttggtaggagtgatcatgatgtttttgtctctaggagttggcgccgaccagggctgcgcgataaatttcggtcaatgtgtgaatcttaccacccgaacccagcttcctcccgcctacacgaattcattcacgcggggtgtttattacccggataaagttttccggtccagtgtcctgcattcaacccaagacctctttctgccatttttttctaacgtgacgtggttccatgctatccatgtaagcggaaccaacggaaccaaacggttcgataatccggttctcccattcaacgatggggtttacttcgcatctacagaaaaatctaacataattagaggatggatttttgggactacgcttgacagtaagacccaatcactcttgatcgtgaacaatgcaaccaatgtagtaattaaggtttgcgagtttcaattttgtaatgatccatttttgggggtttattaccacaaaaacaataaatcctggatggaatccgaattcagagtgtatagcagcgctaacaattgcacatttgagtacgtgtcacaaccttttcttatggatcttgagggcaagcaagggaacttcaaaaatttgagggagttcgttttcaagaacatcgacggatactttaagatctattctaaacacacccccattaacttggtgcgagatttgcctcaaggcttctctgcacttgaaccgttggtggatcttcccattggcattaatattactcggttccagactttgttggcactgcatcgctcctatctcacgcccggagacagttcatctggatggactgcgggggctgccgcgtattacgtgggatacctgcagccgcgcacatttcttcttaaatacaacgagaacgggaccatcacagatgcagtggattgcgctcttgaccccctctccgaaacaaaatgtacgctcaagtctttcactgtagagaaagggatttatcagacatccaatttccgagtccagccaacagagagtatagtgcggttccctaacatcacaaatctttgtccgttcggggaagttttcaacgctacacgcttcgcaagtgtatacgcttggaatagaaagaggatctctaattgtgtggcagattactctgtgctctacaattccgcatctttctcaaccttcaagtgttacggagtttcacctacgaagctgaacgacctttgctttactaatgtatatgcagatagttttgtcatcaggggcgatgaagttcgacagatagcgcccggccagacaggaaagatcgcggactacaattataaactccctgatgatttcaccgggtgcgtgatcgcgtggaactctaataacctcgactccaaagtaggcggtaactacaattacttgtatcgattgtttagaaaatcaaaccttaaaccgttcgagcgggatatctctaccgagatataccaagcaggctctacaccgtgcaatggagtcgaaggttttaactgctacttccccttgcaatcatacgggtttcagcctaccaacggtgtaggataccagccttacagggttgtggtactttcatttgagctcctgcacgctcccgcaactgtctgtgggcccaaaaagagcactaaccttgttaaaaataaatgcgtcaactttaacttcaatggcctcactggcaccggcgtgctcactgaaagcaataagaaattccttccttttcagcagtttgggcgagacatagcggataccacggacgcagtacgggatcctcaaacccttgaaatccttgacataacgccttgctcttttgggggagtaagcgtaatcacgcctggaaccaacacctccaatcaggttgctgtgctgtaccaggatgtaaactgcaccgaggtaccggtagccattcacgcggatcagctgactcccacatggcgagtgtattctacaggtagtaatgtgtttcagacccgagcagggtgtttgataggggcggagcacgtcaacaactcatacgagtgcgatatacccattggggctggtatatgtgcatcctaccagacgcagacgaactctcctcgccgcgctcggtctgttgcatctcaatcaattattgcatacactatgtcactgggggctgagaattcagtagcctactctaacaacagcatcgcgattcccactaacttcacaattagtgtgactaccgagatcctgccagtatccatgactaaaactagcgtagattgtactatgtacatctgcggcgattcaactgagtgttcaaacctcctcttgcaatacgggtcattttgtacccaattgaatcgagctctgaccggcatcgcggtcgaacaggacaaaaatactcaagaggtatttgcccaggtgaagcagatttacaaaacaccccctatcaaggatttcgggggcttcaacttcagccagatactgccagacccctcaaaaccgagcaagcgctccttcatagaagatcttcttttcaacaaagttaccctcgcggatgctggtttcattaaacagtatggggactgtctcggcgacattgctgctagagacctcatctgcgcgcaaaagttcaatggacttacggtcctgccccctctcctcactgatgaaatgattgctcaatatacgtccgcgttgttggcgggaactataaccagtgggtggacgttcggcgctggcgccgcgcttcaaatcccatttgcgatgcaaatggcgtatcgcttcaacggcatcggagtaactcaaaacgttctgtacgaaaatcaaaaactcattgcgaaccagtttaattcagcgatcggtaaaatccaggacagcctgagctccacagcgagtgcactcgggaagctccaggatgtggtaaatcagaacgctcaagcgttgaacacactcgtcaagcagctgtcaagtaactttggcgcgatttcatctgtattgaatgacattctctctcgccttgataaggtggaagccgaagtccagattgatcgcctgattactgggcggcttcagtccctccaaacatacgtcactcagcaacttattagagccgccgaaattagggcaagtgcgaatctggccgcgacaaaaatgtctgaatgtgtgctggggcagagcaagagagtcgatttttgcggtaaggggtatcaccttatgtcttttcctcagtctgcccctcacggagtagtgtttctccacgttacgtatgtcccagcccaagagaaaaactttaccactgcgccggctatttgtcatgacggtaaagcacactttccacgcgaaggtgtgttcgtctccaacggcacccactggtttgtaacgcagaggaacttctacgagcctcagataattaccacggacaacacgttcgtctcaggtaactgcgacgtcgtaattggtattgtaaataacaccgtgtacgacccgctccagccggagctggactccttcaaagaggagcttgacaagtattttaagaatcacacttcaccggatgtagacctgggggatatttccggcataaacgcttccgtggttaacatacagaaagagatagatcgactgaacgaggtagcgaaaaacttgaatgagtctttgatagacctgcaagaattgggaaaatatgaacaatatattaagtggccctggtatatttggcttggtttcatagccggtttgattgccatcgtcatggtaactataatgctttgttgcatgacaagttgctgctcttgcctcaaagggtgctgctcctgtggaagttgttgcaagttcgatgaggatgattctgagccagtgcttaagggtgtcaaactgcattatacgaggtcaattgctatgacgtttcttgcggttggaggagttttgctcttcctttcggtcaacgtccatgctgatcaaggatgcgccatcaactttggcaagagagagctcaagtgcggagatggtatcttcatatttagagactctgatgactggctgaacaagtactcatactatccagaagatcctgtgaagcttgcatcaatagtgaaagcctcttttgaagaagggaagtgtggcctaaattcagttgactcc

SEQ ID NO: 4 - protein, 1394 aa, Artificial Sequence
  construct 1
KVIMGAVLIWVGINTRNMTMSMSMILVGVIMMFLSLGVGADQGCAINFGQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYTRSIAMTFLAVGGVLLFLSVNVHADQGCAINFGKRELKCGDGIFIFRDSDDWLNKYSYYPEDPVKLASIVKASFEEGKCGLNSVDS

SEQ ID NO: 5 - DNA, 4182 bp, Artificial Sequence
  Construct 2
aaggtcatcatgggggcggtacttatatgggttggcatcaacacaagaaacatgacaatgtccatgagcatgatcttggtaggagtgatcatgatgtttttgtctctaggagttggcgccgaccagggctgcgcgataaatttcggtcaatgtgtgaatcttaccacccgaacccagcttcctcccgcctacacgaattcattcacgcggggtgtttattacccggataaagttttccggtccagtgtcctgcattcaacccaagacctctttctgccatttttttctaacgtgacgtggttccatgctatccatgtaagcggaaccaacggaaccaaacggttcgataatccggttctcccattcaacgatggggtttacttcgcatctacagaaaaatctaacataattagaggatggatttttgggactacgcttgacagtaagacccaatcactcttgatcgtgaacaatgcaaccaatgtagtaattaaggtttgcgagtttcaattttgtaatgatccatttttgggggtttattaccacaaaaacaataaatcctggatggaatccgaattcagagtgtatagcagcgctaacaattgcacatttgagtacgtgtcacaaccttttcttatggatcttgagggcaagcaagggaacttcaaaaatttgagggagttcgttttcaagaacatcgacggatactttaagatctattctaaacacacccccattaacttggtgcgagatttgcctcaaggcttctctgcacttgaaccgttggtggatcttcccattggcattaatattactcggttccagactttgttggcactgcatcgctcctatctcacgcccggagacagttcatctggatggactgcgggggctgccgcgtattacgtgggatacctgcagccgcgcacatttcttcttaaatacaacgagaacgggaccatcacagatgcagtggattgcgctcttgaccccctctccgaaacaaaatgtacgctcaagtctttcactgtagagaaagggatttatcagacatccaatttccgagtccagccaacagagagtatagtgcggttccctaacatcacaaatctttgtccgttcggggaagttttcaacgctacacgcttcgcaagtgtatacgcttggaatagaaagaggatctctaattgtgtggcagattactctgtgctctacaattccgcatctttctcaaccttcaagtgttacggagtttcacctacgaagctgaacgacctttgctttactaatgtatatgcagatagttttgtcatcaggggcgatgaagttcgacagatagcgcccggccagacaggaaagatcgcggactacaattataaactccctgatgatttcaccgggtgcgtgatcgcgtggaactctaataacctcgactccaaagtaggcggtaactacaattacttgtatcgattgtttagaaaatcaaaccttaaaccgttcgagcgggatatctctaccgagatataccaagcaggctctacaccgtgcaatggagtcgaaggttttaactgctacttccccttgcaatcatacgggtttcagcctaccaacggtgtaggataccagccttacagggttgtggtactttcatttgagctcctgcacgctcccgcaactgtctgtgggcccaaaaagagcactaaccttgttaaaaataaatgcgtcaactttaacttcaatggcctcactggcaccggcgtgctcactgaaagcaataagaaattccttccttttcagcagtttgggcgagacatagcggataccacggacgcagtacgggatcctcaaacccttgaaatccttgacataacgccttgctcttttgggggagtaagcgtaatcacgcctggaaccaacacctccaatcaggttgctgtgctgtaccaggatgtaaactgcaccgaggtaccggtagccattcacgcggatcagctgactcccacatggcgagtgtattctacaggtagtaatgtgtttcagacccgagcagggtgtttgataggggcggagcacgtcaacaactcatacgagtgcgatatacccattggggctggtatatgtgcatcctaccagacgcagacgaactctcctgccgccgctgcgtctgttgcatctcaatcaattattgcatacactatgtcactgggggctgagaattcagtagcctactctaacaacagcatcgcgattcccactaacttcacaattagtgtgactaccgagatcctgccagtatccatgactaaaactagcgtagattgtactatgtacatctgcggcgattcaactgagtgttcaaacctcctcttgcaatacgggtcattttgtacccaattgaatcgagctctgaccggcatcgcggtcgaacaggacaaaaatactcaagaggtatttgcccaggtgaagcagatttacaaaacaccccctatcaaggatttcgggggcttcaacttcagccagatactgccagacccctcaaaaccgagcaagcgctccttcatagaagatcttcttttcaacaaagttaccctcgcggatgctggtttcattaaacagtatggggactgtctcggcgacattgctgctagagacctcatctgcgcgcaaaagttcaatggacttacggtcctgccccctctcctcactgatgaaatgattgctcaatatacgtccgcgttgttggcgggaactataaccagtgggtggacgttcggcgctggcgccgcgcttcaaatcccatttgcgatgcaaatggcgtatcgcttcaacggcatcggagtaactcaaaacgttctgtacgaaaatcaaaaactcattgcgaaccagtttaattcagcgatcggtaaaatccaggacagcctgagctccacagcgagtgcactcgggaagctccaggatgtggtaaatcagaacgctcaagcgttgaacacactcgtcaagcagctgtcaagtaactttggcgcgatttcatctgtattgaatgacattctctctcgccttgataaggtggaagccgaagtccagattgatcgcctgattactgggcggcttcagtccctccaaacatacgtcactcagcaacttattagagccgccgaaattagggcaagtgcgaatctggccgcgacaaaaatgtctgaatgtgtgctggggcagagcaagagagtcgatttttgcggtaaggggtatcaccttatgtcttttcctcagtctgcccctcacggagtagtgtttctccacgttacgtatgtcccagcccaagagaaaaactttaccactgcgccggctatttgtcatgacggtaaagcacactttccacgcgaaggtgtgttcgtctccaacggcacccactggtttgtaacgcagaggaacttctacgagcctcagataattaccacggacaacacgttcgtctcaggtaactgcgacgtcgtaattggtattgtaaataacaccgtgtacgacccgctccagccggagctggactccttcaaagaggagcttgacaagtattttaagaatcacacttcaccggatgtagacctgggggatatttccggcataaacgcttccgtggttaacatacagaaagagatagatcgactgaacgaggtagcgaaaaacttgaatgagtctttgatagacctgcaagaattgggaaaatatgaacaatatattaagtggccctggtatatttggcttggtttcatagccggtttgattgccatcgtcatggtaactataatgctttgttgcatgacaagttgctgctcttgcctcaaagggtgctgctcctgtggaagttgttgcaagttcgatgaggatgattctgagccagtgcttaagggtgtcaaactgcattatacgaggtcaattgctatgacgtttcttgcggttggaggagttttgctcttcctttcggtcaacgtccatgctgatcaaggatgcgccatcaactttggcaagagagagctcaagtgcggagatggtatcttcatatttagagactctgatgactggctgaacaagtactcatactatccagaagatcctgtgaagcttgcatcaatagtgaaagcctcttttgaagaagggaagtgtggcctaaattcagttgactcc

SEQ ID NO: 6 - protein, 1394 aa, Artificial Sequence
  Construct 2
KVIMGAVLIWVGINTRNMTMSMSMILVGVIMMFLSLGVGADQGCAINFGQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPAAAASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYTRSIAMTFLAVGGVLLFLSVNVHADQGCAINFGKRELKCGDGIFIFRDSDDWLNKYSYYPEDPVKLASIVKASFEEGKCGLNSVDS

SEQ ID NO: 7 - DNA, 4182 bp, Artificial Sequence
  Construct 3
aaggtcatcatgggggcggtacttatatgggttggcatcaacacaagaaacatgacaatgtccatgagcatgatcttggtaggagtgatcatgatgtttttgtctctaggagttggcgccgaccagggctgcgcgataaatttcggtcaatgtgtgaatcttaccacccgaacccagcttcctcccgcctacacgaattcattcacgcggggtgtttattacccggataaagttttccggtccagtgtcctgcattcaacccaagacctctttctgccatttttttctaacgtgacgtggttccatgctatccatgtaagcggaaccaacggaaccaaacggttcgataatccggttctcccattcaacgatggggtttacttcgcatctacagaaaaatctaacataattagaggatggatttttgggactacgcttgacagtaagacccaatcactcttgatcgtgaacaatgcaaccaatgtagtaattaaggtttgcgagtttcaattttgtaatgatccatttttgggggtttattaccacaaaaacaataaatcctggatggaatccgaattcagagtgtatagcagcgctaacaattgcacatttgagtacgtgtcacaaccttttcttatggatcttgagggcaagcaagggaacttcaaaaatttgagggagttcgttttcaagaacatcgacggatactttaagatctattctaaacacacccccattaacttggtgcgagatttgcctcaaggcttctctgcacttgaaccgttggtggatcttcccattggcattaatattactcggttccagactttgttggcactgcatcgctcctatctcacgcccggagacagttcatctggatggactgcgggggctgccgcgtattacgtgggatacctgcagccgcgcacatttcttcttaaatacaacgagaacgggaccatcacagatgcagtggattgcgctcttgaccccctctccgaaacaaaatgtacgctcaagtctttcactgtagagaaagggatttatcagacatccaatttccgagtccagccaacagagagtatagtgcggttccctaacatcacaaatctttgtccgttcggggaagttttcaacgctacacgcttcgcaagtgtatacgcttggaatagaaagaggatctctaattgtgtggcagattactctgtgctctacaattccgcatctttctcaaccttcaagtgttacggagtttcacctacgaagctgaacgacctttgctttactaatgtatatgcagatagttttgtcatcaggggcgatgaagttcgacagatagcgcccggccagacaggaaagatcgcggactacaattataaactccctgatgatttcaccgggtgcgtgatcgcgtggaactctaataacctcgactccaaagtaggcggtaactacaattacttgtatcgattgtttagaaaatcaaaccttaaaccgttcgagcgggatatctctaccgagatataccaagcaggctctacaccgtgcaatggagtcgaaggttttaactgctacttccccttgcaatcatacgggtttcagcctaccaacggtgtaggataccagccttacagggttgtggtactttcatttgagctcctgcacgctcccgcaactgtctgtgggcccaaaaagagcactaaccttgttaaaaataaatgcgtcaactttaacttcaatggcctcactggcaccggcgtgctcactgaaagcaataagaaattccttccttttcagcagtttgggcgagacatagcggataccacggacgcagtacgggatcctcaaacccttgaaatccttgacataacgccttgctcttttgggggagtaagcgtaatcacgcctggaaccaacacctccaatcaggttgctgtgctgtaccaggatgtaaactgcaccgaggtaccggtagccattcacgcggatcagctgactcccacatggcgagtgtattctacaggtagtaatgtgtttcagacccgagcagggtgtttgataggggcggagcacgtcaacaactcatacgagtgcgatatacccattggggctggtatatgtgcatcctaccagacgcagacgaactctcctgccgccgctgcgtctgttgcatctcaatcaattattgcatacactatgtcactgggggctgagaattcagtagcctactctaacaacagcatcgcgattcccactaacttcacaattagtgtgactaccgagatcctgccagtatccatgactaaaactagcgtagattgtactatgtacatctgcggcgattcaactgagtgttcaaacctcctcttgcaatacgggtcattttgtacccaattgaatcgagctctgaccggcatcgcggtcgaacaggacaaaaatactcaagaggtatttgcccaggtgaagcagatttacaaaacaccccctatcaaggatttcgggggcttcaacttcagccagatactgccagacccctcaaaaccgagcgcgaactccttcatagaagatcttcttttcaacaaagttaccctcgcggatgctggtttcattaaacagtatggggactgtctcggcgacattgctgctagagacctcatctgcgcgcaaaagttcaatggacttacggtcctgccccctctcctcactgatgaaatgattgctcaatatacgtccgcgttgttggcgggaactataaccagtgggtggacgttcggcgctggcgccgcgcttcaaatcccatttgcgatgcaaatggcgtatcgcttcaacggcatcggagtaactcaaaacgttctgtacgaaaatcaaaaactcattgcgaaccagtttaattcagcgatcggtaaaatccaggacagcctgagctccacagcgagtgcactcgggaagctccaggatgtggtaaatcagaacgctcaagcgttgaacacactcgtcaagcagctgtcaagtaactttggcgcgatttcatctgtattgaatgacattctctctcgccttgataaggtggaagccgaagtccagattgatcgcctgattactgggcggcttcagtccctccaaacatacgtcactcagcaacttattagagccgccgaaattagggcaagtgcgaatctggccgcgacaaaaatgtctgaatgtgtgctggggcagagcaagagagtcgatttttgcggtaaggggtatcaccttatgtcttttcctcagtctgcccctcacggagtagtgtttctccacgttacgtatgtcccagcccaagagaaaaactttaccactgcgccggctatttgtcatgacggtaaagcacactttccacgcgaaggtgtgttcgtctccaacggcacccactggtttgtaacgcagaggaacttctacgagcctcagataattaccacggacaacacgttcgtctcaggtaactgcgacgtcgtaattggtattgtaaataacaccgtgtacgacccgctccagccggagctggactccttcaaagaggagcttgacaagtattttaagaatcacacttcaccggatgtagacctgggggatatttccggcataaacgcttccgtggttaacatacagaaagagatagatcgactgaacgaggtagcgaaaaacttgaatgagtctttgatagacctgcaagaattgggaaaatatgaacaatatattaagtggccctggtatatttggcttggtttcatagccggtttgattgccatcgtcatggtaactataatgctttgttgcatgacaagttgctgctcttgcctcaaagggtgctgctcctgtggaagttgttgcaagttcgatgaggatgattctgagccagtgcttaagggtgtcaaactgcattatacgaggtcaattgctatgacgtttcttgcggttggaggagttttgctcttcctttcggtcaacgtccatgctgatcaaggatgcgccatcaactttggcaagagagagctcaagtgcggagatggtatcttcatatttagagactctgatgactggctgaacaagtactcatactatccagaagatcctgtgaagcttgcatcaatagtgaaagcctcttttgaagaagggaagtgtggcctaaattcagttgactcc

SEQ ID NO: 8 - protein, 1394 aa, Artificial Sequence
  Construct 3
KVIMGAVLIWVGINTRNMTMSMSMILVGVIMMFLSLGVGADQGCAINFGQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPAAAASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSANSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYTRSIAMTFLAVGGVLLFLSVNVHADQGCAINFGKRELKCGDGIFIFRDSDDWLNKYSYYPEDPVKLASIVKASFEEGKCGLNSVDS

SEQ ID NO: 9 - DNA, 2166 bp, Artificial Sequence
  Construct 4
aaggtcatcatgggggcggtacttatatgggttggcatcaacacaagaaacatgacaatgtccatgagcatgatcttggtaggagtgatcatgatgtttttgtctctaggagttggcgccgaccagggctgcgcgataaatttcggttctgttgcatctcaatcaattattgcatacactatgtcactgggggctgagaattcagtagcctactctaacaacagcatcgcgattcccactaacttcacaattagtgtgactaccgagatcctgccagtatccatgactaaaactagcgtagattgtactatgtacatctgcggcgattcaactgagtgttcaaacctcctcttgcaatacgggtcattttgtacccaattgaatcgagctctgaccggcatcgcggtcgaacaggacaaaaatactcaagaggtatttgcccaggtgaagcagatttacaaaacaccccctatcaaggatttcgggggcttcaacttcagccagatactgccagacccctcaaaaccgagcaagcgctccttcatagaagatcttcttttcaacaaagttaccctcgcggatgctggtttcattaaacagtatggggactgtctcggcgacattgctgctagagacctcatctgcgcgcaaaagttcaatggacttacggtcctgccccctctcctcactgatgaaatgattgctcaatatacgtccgcgttgttggcgggaactataaccagtgggtggacgttcggcgctggcgccgcgcttcaaatcccatttgcgatgcaaatggcgtatcgcttcaacggcatcggagtaactcaaaacgttctgtacgaaaatcaaaaactcattgcgaaccagtttaattcagcgatcggtaaaatccaggacagcctgagctccacagcgagtgcactcgggaagctccaggatgtggtaaatcagaacgctcaagcgttgaacacactcgtcaagcagctgtcaagtaactttggcgcgatttcatctgtattgaatgacattctctctcgccttgataaggtggaagccgaagtccagattgatcgcctgattactgggcggcttcagtccctccaaacatacgtcactcagcaacttattagagccgccgaaattagggcaagtgcgaatctggccgcgacaaaaatgtctgaatgtgtgctggggcagagcaagagagtcgatttttgcggtaaggggtatcaccttatgtcttttcctcagtctgcccctcacggagtagtgtttctccacgttacgtatgtcccagcccaagagaaaaactttaccactgcgccggctatttgtcatgacggtaaagcacactttccacgcgaaggtgtgttcgtctccaacggcacccactggtttgtaacgcagaggaacttctacgagcctcagataattaccacggacaacacgttcgtctcaggtaactgcgacgtcgtaattggtattgtaaataacaccgtgtacgacccgctccagccggagctggactccttcaaagaggagcttgacaagtattttaagaatcacacttcaccggatgtagacctgggggatatttccggcataaacgcttccgtggttaacatacagaaagagatagatcgactgaacgaggtagcgaaaaacttgaatgagtctttgatagacctgcaagaattgggaaaatatgaacaatatattaagtggccctggtatatttggcttggtttcatagccggtttgattgccatcgtcatggtaactataatgctttgttgcatgacaagttgctgctcttgcctcaaagggtgctgctcctgtggaagttgttgcaagttcgatgaggatgattctgagccagtgcttaagggtgtcaaactgcattatacgaggtcaattgctatgacgtttcttgcggttggaggagttttgctcttcctttcggtcaacgtccatgctgatcaaggatgcgccatcaactttggcaagagagagctcaagtgcggagatggtatcttcatatttagagactctgatgactggctgaacaagtactcatactatccagaagatcctgtgaagcttgcatcaatagtgaaagcctcttttgaagaagggaagtgtggcctaaattcagttgactcc

SEQ ID NO: 10 - protein, 722 aa, Artificial Sequence
  Construct 4
KVIMGAVLIWVGINTRNMTMSMSMILVGVIMMFLSLGVGADQGCAINFGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYTRSIAMTFLAVGGVLLFLSVNVHADQGCAINFGKRELKCGDGIFIFRDSDDWLNKYSYYPEDPVKLASIVKASFEEGKCGLNSVDS

SEQ ID NO: 11 - DNA, 2601 bp, Artificial Sequence
  Construct 5
aaggtcatcatgggggcggtacttatatgggttggcatcaacacaagaaacatgacaatgtccatgagcatgatcttggtaggagtgatcatgatgtttttgtctctaggagttggcgccgaccagggctgcgcgataaatttcggtcaatgtgtgaatcttaccacccgaacccagcttcctcccgcctacacgaattcattcacgcggggtgtttattacccggataaagttttccggtccagtgtcctgcattcaacccaagacctctttctgccatttttttctaacgtgacgtggttccatgctatccatgtaagcggaaccaacggaaccaaacggttcgataatccggttctcccattcaacgatggggtttacttcgcatctacagaaaaatctaacataattagaggatggatttttgggactacgcttgacagtaagacccaatcactcttgatcgtgaacaatgcaaccaatgtagtaattaaggtttgcgagtttcaattttgtaatgatccatttttgggggtttattaccacaaaaacaataaatcctggatggaatccgaattcagagtgtatagcagcgctaacaattgcacatttgagtacgtgtcacaaccttttcttatggatcttgagggcaagcaagggaacttcaaaaatttgagggagttcgttttcaagaacatcgacggatactttaagatctattctaaacacacccccattaacttggtgcgagatttgcctcaaggcttctctgcacttgaaccgttggtggatcttcccattggcattaatattactcggttccagactttgttggcactgcatcgctcctatctcacgcccggagacagttcatctggatggactgcgggggctgccgcgtattacgtgggatacctgcagccgcgcacatttcttcttaaatacaacgagaacgggaccatcacagatgcagtggattgcgctcttgaccccctctccgaaacaaaatgtacgctcaagtctttcactgtagagaaagggatttatcagacatccaatttccgagtccagccaacagagagtatagtgcggttccctaacatcacaaatctttgtccgttcggggaagttttcaacgctacacgcttcgcaagtgtatacgcttggaatagaaagaggatctctaattgtgtggcagattactctgtgctctacaattccgcatctttctcaaccttcaagtgttacggagtttcacctacgaagctgaacgacctttgctttactaatgtatatgcagatagttttgtcatcaggggcgatgaagttcgacagatagcgcccggccagacaggaaagatcgcggactacaattataaactccctgatgatttcaccgggtgcgtgatcgcgtggaactctaataacctcgactccaaagtaggcggtaactacaattacttgtatcgattgtttagaaaatcaaaccttaaaccgttcgagcgggatatctctaccgagatataccaagcaggctctacaccgtgcaatggagtcgaaggttttaactgctacttccccttgcaatcatacgggtttcagcctaccaacggtgtaggataccagccttacagggttgtggtactttcatttgagctcctgcacgctcccgcaactgtctgtgggcccaaaaagagcactaaccttgttaaaaataaatgcgtcaactttaacttcaatggcctcactggcaccggcgtgctcactgaaagcaataagaaattccttccttttcagcagtttgggcgagacatagcggataccacggacgcagtacgggatcctcaaacccttgaaatccttgacataacgccttgctcttttgggggagtaagcgtaatcacgcctggaaccaacacctccaatcaggttgctgtgctgtaccaggatgtaaactgcaccgaggtaccggtagccattcacgcggatcagctgactcccacatggcgagtgtattctacaggtagtaatgtgtttcagacccgagcagggtgtttgataggggcggagcacgtcaacaactcatacgagtgcgatatacccattggggctggtatatgtgcatcctaccagacgcagacgaactctcctcgccgcgctcggtctgttgcatctcaatcaattattgcatacactatgtcactgggggctgagaattcagtagcctactctaacaacagcatcgcgattcccactaacttcacaattagtgtgggagggatgtcctggatcacacagggacttctgggagctcttctgttgtggatgggaatcaatgcccgtgacaggtcaattgctatgacgtttcttgcggttggaggagttttgctcttcctttcggtcaacgtccatgctgatcaaggatgcgccatcaactttggcaagagagagctcaagtgcggagatggtatcttcatatttagagactctgatgactggctgaacaagtactcatactatccagaagatcctgtgaagcttgcatcaatagtgaaagcctcttttgaagaagggaagtgtggcctaaattcagttgactcc

SEQ ID NO: 12 - protein, 867 aa, Artificial Sequence
  Construct 5
KVIMGAVLIWVGINTRNMTMSMSMILVGVIMMFLSLGVGADQGCAINFGQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVGGMSWITQGLLGALLLWMGINARDRSIAMTFLAVGGVLLFLSVNVHADQGCAINFGKRELKCGDGIFIFRDSDDWLNKYSYYPEDPVKLASIVKASFEEGKCGLNSVDS

SEQ ID NO: 13 - DNA, 2226 bp, Artificial Sequence
  Construct 6
atgtctggtcgtaaagctcagggaaaaaccctgggcgtcaatatggtacgacgaggagttcgcggatccatgtttgttttcttggtcctccttccactggtatcctcacaatgtgtgaatcttaccacccgaacccagcttcctcccgcctacacgaattcattcacgcggggtgtttattacccggataaagttttccggtccagtgtcctgcattcaacccaagacctctttctgccatttttttctaacgtgacgtggttccatgctatccatgtaagcggaaccaacggaaccaaacggttcgataatccggttctcccattcaacgatggggtttacttcgcatctacagaaaaatctaacataattagaggatggatttttgggactacgcttgacagtaagacccaatcactcttgatcgtgaacaatgcaaccaatgtagtaattaaggtttgcgagtttcaattttgtaatgatccatttttgggggtttattaccacaaaaacaataaatcctggatggaatccgaattcagagtgtatagcagcgctaacaattgcacatttgagtacgtgtcacaaccttttcttatggatcttgagggcaagcaagggaacttcaaaaatttgagggagttcgttttcaagaacatcgacggatactttaagatctattctaaacacacccccattaacttggtgcgagatttgcctcaaggcttctctgcacttgaaccgttggtggatcttcccattggcattaatattactcggttccagactttgttggcactgcatcgctcctatctcacgcccggagacagttcatctggatggactgcgggggctgccgcgtattacgtgggatacctgcagccgcgcacatttcttcttaaatacaacgagaacgggaccatcacagatgcagtggattgcgctcttgaccccctctccgaaacaaaatgtacgctcaagtctttcactgtagagaaagggatttatcagacatccaatttccgagtccagccaacagagagtatagtgcggttccctaacatcacaaatctttgtccgttcggggaagttttcaacgctacacgcttcgcaagtgtatacgcttggaatagaaagaggatctctaattgtgtggcagattactctgtgctctacaattccgcatctttctcaaccttcaagtgttacggagtttcacctacgaagctgaacgacctttgctttactaatgtatatgcagatagttttgtcatcaggggcgatgaagttcgacagatagcgcccggccagacaggaaagatcgcggactacaattataaactccctgatgatttcaccgggtgcgtgatcgcgtggaactctaataacctcgactccaaagtaggcggtaactacaattacttgtatcgattgtttagaaaatcaaaccttaaaccgttcgagcgggatatctctaccgagatataccaagcaggctctacaccgtgcaatggagtcgaaggttttaactgctacttccccttgcaatcatacgggtttcagcctaccaacggtgtaggataccagccttacagggttgtggtactttcatttgagctcctgcacgctcccgcaactgtctgtgggcccaaaaagagcactaaccttgttaaaaataaatgcgtcaactttaacttcaatggcctcactggcaccggcgtgctcactgaaagcaataagaaattccttccttttcagcagtttgggcgagacatagcggataccacggacgcagtacgggatcctcaaacccttgaaatccttgacataacgccttgctcttttgggggagtaagcgtaatcacgcctggaaccaacacctccaatcaggttgctgtgctgtaccaggatgtaaactgcaccgaggtaccggtagccattcacgcggatcagctgactcccacatggcgagtgtattctacaggtagtaatgtgtttcagacccgagcagggtgtttgataggggcggagcacgtcaacaactcatacgagtgcgatatacccattggggctggtatatgtgcatcctaccagacgcagacgaactctcctgagggacgcgggtccctgctgacctgtggcgacgtggaggagaaccccggtcctagcggccgcaaagcccagggtaagacactgggcgtgaacatggttcgtcgcggcgtccgg

SEQ ID NO: 14 - protein, 742 aa, Artificial Sequence
  Construct 6
MSGRKAQGKTLGVNMVRRGVRGSMFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPEGRGSLLTCGDVEENPGPSGRKAQGKTLGVNMVRRGVR

SEQ ID NO: 15 - DNA, 2187 bp, Artificial Sequence
  Construct 7
atgtctggtcgtaaagctcagggaaaaaccctgggcgtcaatatggtacgacgaggagttcgcggatcccaatgtgtgaatcttaccacccgaacccagcttcctcccgcctacacgaattcattcacgcggggtgtttattacccggataaagttttccggtccagtgtcctgcattcaacccaagacctctttctgccatttttttctaacgtgacgtggttccatgctatccatgtaagcggaaccaacggaaccaaacggttcgataatccggttctcccattcaacgatggggtttacttcgcatctacagaaaaatctaacataattagaggatggatttttgggactacgcttgacagtaagacccaatcactcttgatcgtgaacaatgcaaccaatgtagtaattaaggtttgcgagtttcaattttgtaatgatccatttttgggggtttattaccacaaaaacaataaatcctggatggaatccgaattcagagtgtatagcagcgctaacaattgcacatttgagtacgtgtcacaaccttttcttatggatcttgagggcaagcaagggaacttcaaaaatttgagggagttcgttttcaagaacatcgacggatactttaagatctattctaaacacacccccattaacttggtgcgagatttgcctcaaggcttctctgcacttgaaccgttggtggatcttcccattggcattaatattactcggttccagactttgttggcactgcatcgctcctatctcacgcccggagacagttcatctggatggactgcgggggctgccgcgtattacgtgggatacctgcagccgcgcacatttcttcttaaatacaacgagaacgggaccatcacagatgcagtggattgcgctcttgaccccctctccgaaacaaaatgtacgctcaagtctttcactgtagagaaagggatttatcagacatccaatttccgagtccagccaacagagagtatagtgcggttccctaacatcacaaatctttgtccgttcggggaagttttcaacgctacacgcttcgcaagtgtatacgcttggaatagaaagaggatctctaattgtgtggcagattactctgtgctctacaattccgcatctttctcaaccttcaagtgttacggagtttcacctacgaagctgaacgacctttgctttactaatgtatatgcagatagttttgtcatcaggggcgatgaagttcgacagatagcgcccggccagacaggaaagatcgcggactacaattataaactccctgatgatttcaccgggtgcgtgatcgcgtggaactctaataacctcgactccaaagtaggcggtaactacaattacttgtatcgattgtttagaaaatcaaaccttaaaccgttcgagcgggatatctctaccgagatataccaagcaggctctacaccgtgcaatggagtcgaaggttttaactgctacttccccttgcaatcatacgggtttcagcctaccaacggtgtaggataccagccttacagggttgtggtactttcatttgagctcctgcacgctcccgcaactgtctgtgggcccaaaaagagcactaaccttgttaaaaataaatgcgtcaactttaacttcaatggcctcactggcaccggcgtgctcactgaaagcaataagaaattccttccttttcagcagtttgggcgagacatagcggataccacggacgcagtacgggatcctcaaacccttgaaatccttgacataacgccttgctcttttgggggagtaagcgtaatcacgcctggaaccaacacctccaatcaggttgctgtgctgtaccaggatgtaaactgcaccgaggtaccggtagccattcacgcggatcagctgactcccacatggcgagtgtattctacaggtagtaatgtgtttcagacccgagcagggtgtttgataggggcggagcacgtcaacaactcatacgagtgcgatatacccattggggctggtatatgtgcatcctaccagacgcagacgaactctcctgagggacgcgggtccctgctgacctgtggcgacgtggaggagaaccccggtcctagcggccgcaaagcccagggtaagacactgggcgtgaacatggttcgtcgcggcgtccgg

SEQ ID NO: 16 - protein, 729 aa, Artificial Sequence
  Construct 7
MSGRKAQGKTLGVNMVRRGVRGSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPEGRGSLLTCGDVEENPGPSGRKAQGKTLGVNMVRRGVR

SEQ ID NO: 17 - DNA, 1764 bp, SARS-CoV-2
tctgttgcatctcaatcaattattgcatacactatgtcactgggggctgagaattcagtagcctactctaacaacagcatcgcgattcccactaacttcacaattagtgtgactaccgagatcctgccagtatccatgactaaaactagcgtagattgtactatgtacatctgcggcgattcaactgagtgttcaaacctcctcttgcaatacgggtcattttgtacccaattgaatcgagctctgaccggcatcgcggtcgaacaggacaaaaatactcaagaggtatttgcccaggtgaagcagatttacaaaacaccccctatcaaggatttcgggggcttcaacttcagccagatactgccagacccctcaaaaccgagcaagcgctccttcatagaagatcttcttttcaacaaagttaccctcgcggatgctggtttcattaaacagtatggggactgtctcggcgacattgctgctagagacctcatctgcgcgcaaaagttcaatggacttacggtcctgccccctctcctcactgatgaaatgattgctcaatatacgtccgcgttgttggcgggaactataaccagtgggtggacgttcggcgctggcgccgcgcttcaaatcccatttgcgatgcaaatggcgtatcgcttcaacggcatcggagtaactcaaaacgttctgtacgaaaatcaaaaactcattgcgaaccagtttaattcagcgatcggtaaaatccaggacagcctgagctccacagcgagtgcactcgggaagctccaggatgtggtaaatcagaacgctcaagcgttgaacacactcgtcaagcagctgtcaagtaactttggcgcgatttcatctgtattgaatgacattctctctcgccttgataaggtggaagccgaagtccagattgatcgcctgattactgggcggcttcagtccctccaaacatacgtcactcagcaacttattagagccgccgaaattagggcaagtgcgaatctggccgcgacaaaaatgtctgaatgtgtgctggggcagagcaagagagtcgatttttgcggtaaggggtatcaccttatgtcttttcctcagtctgcccctcacggagtagtgtttctccacgttacgtatgtcccagcccaagagaaaaactttaccactgcgccggctatttgtcatgacggtaaagcacactttccacgcgaaggtgtgttcgtctccaacggcacccactggtttgtaacgcagaggaacttctacgagcctcagataattaccacggacaacacgttcgtctcaggtaactgcgacgtcgtaattggtattgtaaataacaccgtgtacgacccgctccagccggagctggactccttcaaagaggagcttgacaagtattttaagaatcacacttcaccggatgtagacctgggggatatttccggcataaacgcttccgtggttaacatacagaaagagatagatcgactgaacgaggtagcgaaaaacttgaatgagtctttgatagacctgcaagaattgggaaaatatgaacaatatattaagtggccctggtatatttggcttggtttcatagccggtttgattgccatcgtcatggtaactataatgctttgttgcatgacaagttgctgctcttgcctcaaagggtgctgctcctgtggaagttgttgcaagttcgatgaggatgattctgagccagtgcttaagggtgtcaaactgcattatacg

SEQ ID NO: 18 - DNA, 2010 bp, SARS-CoV-2
gtgaatcttaccacccgaacccagcttcctcccgcctacacgaattcattcacgcggggtgtttattacccggataaagttttccggtccagtgtcctgcattcaacccaagacctctttctgccatttttttctaacgtgacgtggttccatgctatccatgtaagcggaaccaacggaaccaaacggttcgataatccggttctcccattcaacgatggggtttacttcgcatctacagaaaaatctaacataattagaggatggatttttgggactacgcttgacagtaagacccaatcactcttgatcgtgaacaatgcaaccaatgtagtaattaaggtttgcgagtttcaattttgtaatgatccatttttgggggtttattaccacaaaaacaataaatcctggatggaatccgaattcagagtgtatagcagcgctaacaattgcacatttgagtacgtgtcacaaccttttcttatggatcttgagggcaagcaagggaacttcaaaaatttgagggagttcgttttcaagaacatcgacggatactttaagatctattctaaacacacccccattaacttggtgcgagatttgcctcaaggcttctctgcacttgaaccgttggtggatcttcccattggcattaatattactcggttccagactttgttggcactgcatcgctcctatctcacgcccggagacagttcatctggatggactgcgggggctgccgcgtattacgtgggatacctgcagccgcgcacatttcttcttaaatacaacgagaacgggaccatcacagatgcagtggattgcgctcttgaccccctctccgaaacaaaatgtacgctcaagtctttcactgtagagaaagggatttatcagacatccaatttccgagtccagccaacagagagtatagtgcggttccctaacatcacaaatctttgtccgttcggggaagttttcaacgctacacgcttcgcaagtgtatacgcttggaatagaaagaggatctctaattgtgtggcagattactctgtgctctacaattccgcatctttctcaaccttcaagtgttacggagtttcacctacgaagctgaacgacctttgctttactaatgtatatgcagatagttttgtcatcaggggcgatgaagttcgacagatagcgcccggccagacaggaaagatcgcggactacaattataaactccctgatgatttcaccgggtgcgtgatcgcgtggaactctaataacctcgactccaaagtaggcggtaactacaattacttgtatcgattgtttagaaaatcaaaccttaaaccgttcgagcgggatatctctaccgagatataccaagcaggctctacaccgtgcaatggagtcgaaggttttaactgctacttccccttgcaatcatacgggtttcagcctaccaacggtgtaggataccagccttacagggttgtggtactttcatttgagctcctgcacgctcccgcaactgtctgtgggcccaaaaagagcactaaccttgttaaaaataaatgcgtcaactttaacttcaatggcctcactggcaccggcgtgctcactgaaagcaataagaaattccttccttttcagcagtttgggcgagacatagcggataccacggacgcagtacgggatcctcaaacccttgaaatccttgacataacgccttgctcttttgggggagtaagcgtaatcacgcctggaaccaacacctccaatcaggttgctgtgctgtaccaggatgtaaactgcaccgaggtaccggtagccattcacgcggatcagctgactcccacatggcgagtgtattctacaggtagtaatgtgtttcagacccgagcagggtgtttgataggggcggagcacgtcaacaactcatacgagtgcgatatacccattggggctggtatatgtgcatcctaccagacgcagacgaactctcctcgccgcgctcgg

SEQ ID NO: 19 - DNA, 3774 bp, SARS-CoV-2
gtgaatcttaccacccgaacccagcttcctcccgcctacacgaattcattcacgcggggtgtttattacccggataaagttttccggtccagtgtcctgcattcaacccaagacctctttctgccatttttttctaacgtgacgtggttccatgctatccatgtaagcggaaccaacggaaccaaacggttcgataatccggttctcccattcaacgatggggtttacttcgcatctacagaaaaatctaacataattagaggatggatttttgggactacgcttgacagtaagacccaatcactcttgatcgtgaacaatgcaaccaatgtagtaattaaggtttgcgagtttcaattttgtaatgatccatttttgggggtttattaccacaaaaacaataaatcctggatggaatccgaattcagagtgtatagcagcgctaacaattgcacatttgagtacgtgtcacaaccttttcttatggatcttgagggcaagcaagggaacttcaaaaatttgagggagttcgttttcaagaacatcgacggatactttaagatctattctaaacacacccccattaacttggtgcgagatttgcctcaaggcttctctgcacttgaaccgttggtggatcttcccattggcattaatattactcggttccagactttgttggcactgcatcgctcctatctcacgcccggagacagttcatctggatggactgcgggggctgccgcgtattacgtgggatacctgcagccgcgcacatttcttcttaaatacaacgagaacgggaccatcacagatgcagtggattgcgctcttgaccccctctccgaaacaaaatgtacgctcaagtctttcactgtagagaaagggatttatcagacatccaatttccgagtccagccaacagagagtatagtgcggttccctaacatcacaaatctttgtccgttcggggaagttttcaacgctacacgcttcgcaagtgtatacgcttggaatagaaagaggatctctaattgtgtggcagattactctgtgctctacaattccgcatctttctcaaccttcaagtgttacggagtttcacctacgaagctgaacgacctttgctttactaatgtatatgcagatagttttgtcatcaggggcgatgaagttcgacagatagcgcccggccagacaggaaagatcgcggactacaattataaactccctgatgatttcaccgggtgcgtgatcgcgtggaactctaataacctcgactccaaagtaggcggtaactacaattacttgtatcgattgtttagaaaatcaaaccttaaaccgttcgagcgggatatctctaccgagatataccaagcaggctctacaccgtgcaatggagtcgaaggttttaactgctacttccccttgcaatcatacgggtttcagcctaccaacggtgtaggataccagccttacagggttgtggtactttcatttgagctcctgcacgctcccgcaactgtctgtgggcccaaaaagagcactaaccttgttaaaaataaatgcgtcaactttaacttcaatggcctcactggcaccggcgtgctcactgaaagcaataagaaattccttccttttcagcagtttgggcgagacatagcggataccacggacgcagtacgggatcctcaaacccttgaaatccttgacataacgccttgctcttttgggggagtaagcgtaatcacgcctggaaccaacacctccaatcaggttgctgtgctgtaccaggatgtaaactgcaccgaggtaccggtagccattcacgcggatcagctgactcccacatggcgagtgtattctacaggtagtaatgtgtttcagacccgagcagggtgtttgataggggcggagcacgtcaacaactcatacgagtgcgatatacccattggggctggtatatgtgcatcctaccagacgcagacgaactctcctcgccgcgctcggtctgttgcatctcaatcaattattgcatacactatgtcactgggggctgagaattcagtagcctactctaacaacagcatcgcgattcccactaacttcacaattagtgtgactaccgagatcctgccagtatccatgactaaaactagcgtagattgtactatgtacatctgcggcgattcaactgagtgttcaaacctcctcttgcaatacgggtcattttgtacccaattgaatcgagctctgaccggcatcgcggtcgaacaggacaaaaatactcaagaggtatttgcccaggtgaagcagatttacaaaacaccccctatcaaggatttcgggggcttcaacttcagccagatactgccagacccctcaaaaccgagcaagcgctccttcatagaagatcttcttttcaacaaagttaccctcgcggatgctggtttcattaaacagtatggggactgtctcggcgacattgctgctagagacctcatctgcgcgcaaaagttcaatggacttacggtcctgccccctctcctcactgatgaaatgattgctcaatatacgtccgcgttgttggcgggaactataaccagtgggtggacgttcggcgctggcgccgcgcttcaaatcccatttgcgatgcaaatggcgtatcgcttcaacggcatcggagtaactcaaaacgttctgtacgaaaatcaaaaactcattgcgaaccagtttaattcagcgatcggtaaaatccaggacagcctgagctccacagcgagtgcactcgggaagctccaggatgtggtaaatcagaacgctcaagcgttgaacacactcgtcaagcagctgtcaagtaactttggcgcgatttcatctgtattgaatgacattctctctcgccttgataaggtggaagccgaagtccagattgatcgcctgattactgggcggcttcagtccctccaaacatacgtcactcagcaacttattagagccgccgaaattagggcaagtgcgaatctggccgcgacaaaaatgtctgaatgtgtgctggggcagagcaagagagtcgatttttgcggtaaggggtatcaccttatgtcttttcctcagtctgcccctcacggagtagtgtttctccacgttacgtatgtcccagcccaagagaaaaactttaccactgcgccggctatttgtcatgacggtaaagcacactttccacgcgaaggtgtgttcgtctccaacggcacccactggtttgtaacgcagaggaacttctacgagcctcagataattaccacggacaacacgttcgtctcaggtaactgcgacgtcgtaattggtattgtaaataacaccgtgtacgacccgctccagccggagctggactccttcaaagaggagcttgacaagtattttaagaatcacacttcaccggatgtagacctgggggatatttccggcataaacgcttccgtggttaacatacagaaagagatagatcgactgaacgaggtagcgaaaaacttgaatgagtctttgatagacctgcaagaattgggaaaatatgaacaatatattaagtggccctggtatatttggcttggtttcatagccggtttgattgccatcgtcatggtaactataatgctttgttgcatgacaagttgctgctcttgcctcaaagggtgctgctcctgtggaagttgttgcaagttcgatgaggatgattctgagccagtgcttaagggtgtcaaactgcattatacg

SEQ ID NO: 20 - DNA, 39 bp, SARS-CoV2
atgtttgttttcttggtcctccttccactggtatcctca

SEQ ID NO: 21 - DNA, 12 bp, SARS-CoV2
cgccgcgctcgg

SEQ ID NO: 22 - DNA, 12 bp, Artificial Sequence
  mutated S1/S2 cleavage site
gccgccgctgcg

SEQ ID NO: 23 - protein, 4 aa, SARS-CoV2
RRAR

SEQ ID NO: 24 - protein, 4 aa, Artificial Sequence
  mutated S1/S2 cleavage site
AAAA

SEQ ID NO: 25 - protein, 21 aa, Artificial Sequence
  N-terminal part of the capsid protein of the flavivirus
MSGRKAQGKTLGVNMVRRGVR

SEQ ID NO: 26 - DNA, 63 bp, Artificial Sequence
  N-terminal part of the capsid protein of the flavivirus
atgtctggtcgtaaagctcagggaaaaaccctgggcgtcaatatggtacgacgaggagttcgc

SEQ ID NO: 27 - protein, 18 aa, Thosea asigna virus
EGRGSLLTCGDVEENPGP

SEQ ID NO: 28 - protein, 24 aa, Foot-and-mouth disease virus
APVKQTLNFDLLKLAGDVESNPGP

SEQ ID NO: 29 - protein, 19 aa, porcine teschovirus-1
ATNFSLLKQAGDVEENPGP

SEQ ID NO: 30 - protein, 20 aa, equine rhinitis A virus
QCTNYALLKLAGDVESNPGP

SEQ ID NO: 31 - protein, 13 aa, Artificial Sequence
  consensus sequence
  SITE (2)..(4): X can be any amino acid
  SITE (9)..(9): X can be any amino acid
LXXXGDVEXNPGP

SEQ ID NO: 32 - protein, 8 aa, Artificial Sequence
  consensus sequence
  SITE (2)..(2): X can be any amino acid
  SITE (4)..(4): X can be any amino acid
DXEXNPGP

SEQ ID NO: 33 - protein, 4 aa, Artificial Sequence
  T2A ′cleavage′ site
NPGP

SEQ ID NO: 34 - protein, 5 aa, Artificial Sequence
  T2A ′cleavage′ site
NPGPS

SEQ ID NO: 35 - protein, 9 aa, Artificial Sequence
  fragment of the NS1 protein of the flavivirus
DQGCAINFG

SEQ ID NO: 36 - DNA, 27 bp, Artificial Sequence
  fragment of the NS1 protein of the flavivirus
gaccagggctgcgcgataaatttcggt

SEQ ID NO: 37 - DNA, 69 bp, West Nile virus
aggtcaattgctatgacgtttcttgcggttggaggagttttgctcttcctttcggtcaacgtccatgct

SEQ ID NO: 38 - DNA, 27 bp, Artificial Sequence
  NS1 signal peptide
gaccagggctgcgcgataaatttcggt

SEQ ID NO: 39 - DNA, 33 bp, Artificial Sequence
  combination signal peptides
gaccagggctgcgcgataaatttcggtcaatgt

SEQ ID NO: 40 - DNA, 20 bp, Artificial Sequence
  SARS-CoV-2 primer 1
ttacaaacattggccgcaaa

SEQ ID NO: 41 - DNA, 18 bp, Artificial Sequence
  SARS-CoV-2 primer 2
gcgcgacattccgaagaa

SEQ ID NO: 42 - DNA, 23 bp, Artificial Sequence
  SARS-CoV-2 probe
  misc_feature (1)..(1): coupled to fluorescein (FAM)
  misc_feature (23)..(23): coupled to BHQ1
acaatttgcccccagcgcttcag

SEQ ID NO: 43 - DNA, 21 bp, Artificial Sequence
  Hamster ACE2 primer 1
gggaactgtcaaagggtacag

SEQ ID NO: 44 - DNA, 22 bp, Artificial Sequence
  Hamster ACE2 primer 2
cccttcctacatcagtcctact

SEQ ID NO: 45 - DNA, 24 bp, Artificial Sequence
  Hamster ACE2 probe
  misc_feature (1)..(1): coupled to FAM
  misc_feature (24)..(24): coupled to ZEN/IABkFQ
tccctgctcatttgcttggtgaca

SEQ ID NO: 46 - DNA, 18 bp, Artificial Sequence
  Hamster ACTB primer 1
ggccaggtcatcaccatt

SEQ ID NO: 47 - DNA, 24 bp, Artificial Sequence
  Hamster ACTB primer 2
gagttgaatgtagtttcgtggatg

SEQ ID NO: 48 - DNA, 25 bp, Artificial Sequence
  Hamster ACTB probe
  misc_feature (1)..(1): coupled to Cy5
  misc_feature (25)..(25): coupled to IBRQ
tttccagccttccttcctgggtatg

SEQ ID NO: 49 - DNA, 21 bp, Artificial Sequence
  Hamster IFN gamma primer 1
tttctccatgctgctgttgaa

SEQ ID NO: 50 - DNA, 20 bp, Artificial Sequence
  Hamster IFN gamma  primer 2
ggccatccagaggagcatag

SEQ ID NO: 51 - DNA, 30 bp, Artificial Sequence
  Hamster IFN gamma probe
  misc_feature (1)..(1): coupled to fluorescein (FAM)
  misc_feature (30)..(30): coupled to ZEN/IABkFQ
caccatcaaggcagacctgtttgctaactt

SEQ ID NO: 52 - DNA, 20 bp, Artificial Sequence
  Hamster IFN lambda primer 1
cccaccagatgcaaaggatt

SEQ ID NO: 53 - DNA, 22 bp, Artificial Sequence
  Hamster IFN lambda primer 2
cttgagcagccactcttctatg

SEQ ID NO: 54 - DNA, 24 bp, Artificial Sequence
  Hamster IFN lambda probe
  misc_feature (1)..(1): coupled to fluorescein (FAM)
  misc_feature (24)..(24): coupled to ZEN/IABkFQ
acatagcccggttcaagtctctgc

SEQ ID NO: 55 - DNA, 25 bp, Artificial Sequence
  Hamster IL-2 primer 1
aagctcctgtaagtccagcagtaac

SEQ ID NO: 56 - DNA, 22 bp, Artificial Sequence
  Hamster IL-2  primer 2
gtgcacccacttcaagctctaa

SEQ ID NO: 57 - DNA, 24 bp, Artificial Sequence
  Hamster IL-2 probe
  misc_feature (1)..(1): coupled to fluorescein (FAM)
  misc_feature (24)..(24): coupled to ZEN/IABkFQ“
aggaaacccagcagcacctcgagc

SEQ ID NO: 58 - DNA, 24 bp, Artificial Sequence
  Hamster IL-4 primer 1
gggtcacctcatgttggaaataaa

SEQ ID NO: 59 - DNA, 22 bp, Artificial Sequence
  Hamster IL-4 primer 2
ccacggagaaagacctcatctg

SEQ ID NO: 60 - DNA, 26 bp, Artificial Sequence
  Hamster IL-4 probe
  misc_feature (1)..(1): coupled to fluorescein (FAM)
  misc_feature (26)..(26): coupled to ZEN/IABkFQ
cagggcttcccaggtgcttcgcaagt

SEQ ID NO: 61 - DNA, 23 bp, Artificial Sequence
  Hamster IL-6 primer 1
ggtatgctaaggcacagcacact

SEQ ID NO: 62 - DNA, 23 bp, Artificial Sequence
  Hamster IL-6 primer 2
cctgaaagcacttgaagaattcc

SEQ ID NO: 63 - DNA, 30 bp, Artificial Sequence
  Hamster IL-6 probe
  misc_feature (1)..(1): coupled to fluorescein (FAM)
  misc_feature (30)..(30): coupled to ZEN/IABkFQ
agaagtcaccatgaggtctactcggcaaaa

SEQ ID NO: 64 - DNA, 19 bp, Artificial Sequence
  Hamster IL-10 primer 1
ttctggcccgtggttctct

SEQ ID NO: 65 - DNA, 24 bp, Artificial Sequence
  Hamster IL-10 primer 2
gttgccaaaccttatcagaaatga

SEQ ID NO: 66 - DNA, 31 bp, Artificial Sequence
  Hamster IL-10 probe
  misc_feature (1)..(1): coupled to fluorescein (FAM)
  misc_feature (31)..(31): coupled to ZEN/IABkFQ
cagttttacctggtagaagtgatgccccagg

SEQ ID NO: 67 - DNA, 21 bp, Artificial Sequence
  Hamster IP-10 primer 1
gccattcatccacagttgaca

SEQ ID NO: 68 - DNA, 22 bp, Artificial Sequence
  Hamster IP-10 primer 2
catggtgctgacagtggagtct

SEQ ID NO: 69 - DNA, 20 bp, Artificial Sequence
  Hamster IP-10 probe
  misc_feature (1)..(1): coupled to fluorescein (FAM)
  misc_feature (20)..(20): coupled to ZEN/IABkFQ
cgtcccgagccagccaacga

SEQ ID NO: 70 - DNA, 20 bp, Artificial Sequence
  Hamster MX2 primer 1
ccagtaatgtggacattgcc

SEQ ID NO: 71 - DNA, 23 bp, Artificial Sequence
  Hamster MX2  primer 2
catcaacgaccttgtcttcagta

SEQ ID NO: 72 - DNA, 24 bp, Artificial Sequence
  Hamster MX2  probe
  misc_feature (1)..(1): coupled to fluorescein (FAM)
  misc_feature (24)..(24): coupled to ZEN/IABkFQ
tgtccaccagatcaggcttggtca

SEQ ID NO: 73 - DNA, 24 bp, Artificial Sequence
  Hamster TNF primer 1
agctggttgtctttgagagacatg

SEQ ID NO: 74 - DNA, 19 bp, Artificial Sequence
  Hamster TNF primer 2
ggagtggctgagccatcgt

SEQ ID NO: 75 - DNA, 21 bp, Artificial Sequence
  Hamster  TNF  probe
  misc_feature (1)..(1): coupled to fluorescein (FAM)
  misc_feature (21)..(21): coupled to ZEN/IABkFQ
ccaatgccctcctggccaacg

SEQ ID NO: 76 - DNA, 23 bp, Artificial Sequence
  Mouse GADPH primer 1
gtggagtcatacggaacatgtag

SEQ ID NO: 77 - DNA, 19 bp, Artificial Sequence
  Mouse GADPH primer 2
aatggtgaaggtcggtgtg

SEQ ID NO: 78 - DNA, 20 bp, Artificial Sequence
  Mouse GADPH probe
  misc_feature (1)..(1): coupled to 56-FAM
  misc_feature (9)..(9): coupled to internal ZEN quencher
  misc_feature (20)..(20): coupled to ZEN/IABkFQ
tgcaaatggcagccctggtg

SEQ ID NO: 79 - DNA, 22 bp, Artificial Sequence
  Mouse Tbx21 primer 1
caagaccacatccacaaacatc

SEQ ID NO: 80 - DNA, 19 bp, Artificial Sequence
  Mouse Tbx21 primer 2
ttcaaccagcaccagacag

SEQ ID NO: 81 - DNA, 24 bp, Artificial Sequence
  Mouse Tbx21 probe
  misc_feature (1)..(1): coupled to 56-FAM
  misc_feature (9)..(9): coupled to internal ZEN quencher
  misc_feature (24)..(24): coupled to 3IABkFQ
tcactaagcaaggacggcgaatgt

SEQ ID NO: 82 - DNA, 19 bp, Artificial Sequence
  Mouse GATA3 primer 1
gtccccattagcgttcctc

SEQ ID NO: 83 - DNA, 20 bp, Artificial Sequence
  Mouse GATA3 primer 2
ccttatcaagcccaagcgaa

SEQ ID NO: 84 - DNA, 21 bp, Artificial Sequence
  Mouse GATA3 probe
  misc_feature (1)..(1): coupled to 56-FAM
  misc_feature (9)..(9): coupled to internal ZEN quencher
  misc_feature (21)..(21): coupled to 3IABkFQ
tgtccctgctctccttgctgc

SEQ ID NO: 85 - DNA, 19 bp, Artificial Sequence
  Mouse RORO primer 1
gaggtgctggaagatctgc

SEQ ID NO: 86 - DNA, 21 bp, Artificial Sequence
  Mouse RORO primer 2
tctgcaagactcatcgacaag

SEQ ID NO: 87 - DNA, 22 bp, Artificial Sequence
  Mouse RORO probe
  misc_feature (1)..(1): coupled to 56-FAM
  misc_feature (9)..(9): coupled to internal ZEN quencher
  misc_feature (22)..(22): coupled to 3IABkFQ
ctagccaagctgccacccaaag

SEQ ID NO: 88 - DNA, 21 bp, Artificial Sequence
  Mouse FOXP3 primer 1
ctgtcttccaagtctcgtctg

SEQ ID NO: 89 - DNA, 21 bp, Artificial Sequence
  Mouse FOXP3 primer 2
ctggtctctgcaggtttagtg

SEQ ID NO: 90 - DNA, 23 bp, Artificial Sequence
  Mouse FOXP3 probe
  misc_feature (1)..(1): coupled to 56-FAM
  misc_feature (9)..(9): coupled to internal ZEN quencher
  misc_feature (23)..(23): coupled 3IABkFQ
ctgtgcctggtatatgctcccgg

SEQ ID NO: 91 - protein, 4 aa, Artificial Sequence
  mutated S1/S2 cleavage site
SGAG

SEQ ID NO: 92 - protein, 4 aa, Artificial Sequence
  mutated S1/S2 cleavage site
GSAS

SEQ ID NO: 93 - DNA, 3876 bp, Artificial Sequence
  fragment of construct 1
gaccagggctgcgcgataaatttcggtcaatgtgtgaatcttaccacccgaacccagcttcctcccgcctacacgaattcattcacgcggggtgtttattacccggataaagttttccggtccagtgtcctgcattcaacccaagacctctttctgccatttttttctaacgtgacgtggttccatgctatccatgtaagcggaaccaacggaaccaaacggttcgataatccggttctcccattcaacgatggggtttacttcgcatctacagaaaaatctaacataattagaggatggatttttgggactacgcttgacagtaagacccaatcactcttgatcgtgaacaatgcaaccaatgtagtaattaaggtttgcgagtttcaattttgtaatgatccatttttgggggtttattaccacaaaaacaataaatcctggatggaatccgaattcagagtgtatagcagcgctaacaattgcacatttgagtacgtgtcacaaccttttcttatggatcttgagggcaagcaagggaacttcaaaaatttgagggagttcgttttcaagaacatcgacggatactttaagatctattctaaacacacccccattaacttggtgcgagatttgcctcaaggcttctctgcacttgaaccgttggtggatcttcccattggcattaatattactcggttccagactttgttggcactgcatcgctcctatctcacgcccggagacagttcatctggatggactgcgggggctgccgcgtattacgtgggatacctgcagccgcgcacatttcttcttaaatacaacgagaacgggaccatcacagatgcagtggattgcgctcttgaccccctctccgaaacaaaatgtacgctcaagtctttcactgtagagaaagggatttatcagacatccaatttccgagtccagccaacagagagtatagtgcggttccctaacatcacaaatctttgtccgttcggggaagttttcaacgctacacgcttcgcaagtgtatacgcttggaatagaaagaggatctctaattgtgtggcagattactctgtgctctacaattccgcatctttctcaaccttcaagtgttacggagtttcacctacgaagctgaacgacctttgctttactaatgtatatgcagatagttttgtcatcaggggcgatgaagttcgacagatagcgcccggccagacaggaaagatcgcggactacaattataaactccctgatgatttcaccgggtgcgtgatcgcgtggaactctaataacctcgactccaaagtaggcggtaactacaattacttgtatcgattgtttagaaaatcaaaccttaaaccgttcgagcgggatatctctaccgagatataccaagcaggctctacaccgtgcaatggagtcgaaggttttaactgctacttccccttgcaatcatacgggtttcagcctaccaacggtgtaggataccagccttacagggttgtggtactttcatttgagctcctgcacgctcccgcaactgtctgtgggcccaaaaagagcactaaccttgttaaaaataaatgcgtcaactttaacttcaatggcctcactggcaccggcgtgctcactgaaagcaataagaaattccttccttttcagcagtttgggcgagacatagcggataccacggacgcagtacgggatcctcaaacccttgaaatccttgacataacgccttgctcttttgggggagtaagcgtaatcacgcctggaaccaacacctccaatcaggttgctgtgctgtaccaggatgtaaactgcaccgaggtaccggtagccattcacgcggatcagctgactcccacatggcgagtgtattctacaggtagtaatgtgtttcagacccgagcagggtgtttgataggggcggagcacgtcaacaactcatacgagtgcgatatacccattggggctggtatatgtgcatcctaccagacgcagacgaactctcctcgccgcgctcggtctgttgcatctcaatcaattattgcatacactatgtcactgggggctgagaattcagtagcctactctaacaacagcatcgcgattcccactaacttcacaattagtgtgactaccgagatcctgccagtatccatgactaaaactagcgtagattgtactatgtacatctgcggcgattcaactgagtgttcaaacctcctcttgcaatacgggtcattttgtacccaattgaatcgagctctgaccggcatcgcggtcgaacaggacaaaaatactcaagaggtatttgcccaggtgaagcagatttacaaaacaccccctatcaaggatttcgggggcttcaacttcagccagatactgccagacccctcaaaaccgagcaagcgctccttcatagaagatcttcttttcaacaaagttaccctcgcggatgctggtttcattaaacagtatggggactgtctcggcgacattgctgctagagacctcatctgcgcgcaaaagttcaatggacttacggtcctgccccctctcctcactgatgaaatgattgctcaatatacgtccgcgttgttggcgggaactataaccagtgggtggacgttcggcgctggcgccgcgcttcaaatcccatttgcgatgcaaatggcgtatcgcttcaacggcatcggagtaactcaaaacgttctgtacgaaaatcaaaaactcattgcgaaccagtttaattcagcgatcggtaaaatccaggacagcctgagctccacagcgagtgcactcgggaagctccaggatgtggtaaatcagaacgctcaagcgttgaacacactcgtcaagcagctgtcaagtaactttggcgcgatttcatctgtattgaatgacattctctctcgccttgataaggtggaagccgaagtccagattgatcgcctgattactgggcggcttcagtccctccaaacatacgtcactcagcaacttattagagccgccgaaattagggcaagtgcgaatctggccgcgacaaaaatgtctgaatgtgtgctggggcagagcaagagagtcgatttttgcggtaaggggtatcaccttatgtcttttcctcagtctgcccctcacggagtagtgtttctccacgttacgtatgtcccagcccaagagaaaaactttaccactgcgccggctatttgtcatgacggtaaagcacactttccacgcgaaggtgtgttcgtctccaacggcacccactggtttgtaacgcagaggaacttctacgagcctcagataattaccacggacaacacgttcgtctcaggtaactgcgacgtcgtaattggtattgtaaataacaccgtgtacgacccgctccagccggagctggactccttcaaagaggagcttgacaagtattttaagaatcacacttcaccggatgtagacctgggggatatttccggcataaacgcttccgtggttaacatacagaaagagatagatcgactgaacgaggtagcgaaaaacttgaatgagtctttgatagacctgcaagaattgggaaaatatgaacaatatattaagtggccctggtatatttggcttggtttcatagccggtttgattgccatcgtcatggtaactataatgctttgttgcatgacaagttgctgctcttgcctcaaagggtgctgctcctgtggaagttgttgcaagttcgatgaggatgattctgagccagtgcttaagggtgtcaaactgcattatacgaggtcaattgctatgacgtttcttgcggttggaggagttttgctcttcctttcggtcaacgtccatgct

SEQ ID NO: 94 - DNA, 3876 bp, Artificial Sequence
  fragment of construct 2
gaccagggctgcgcgataaatttcggtcaatgtgtgaatcttaccacccgaacccagcttcctcccgcctacacgaattcattcacgcggggtgtttattacccggataaagttttccggtccagtgtcctgcattcaacccaagacctctttctgccatttttttctaacgtgacgtggttccatgctatccatgtaagcggaaccaacggaaccaaacggttcgataatccggttctcccattcaacgatggggtttacttcgcatctacagaaaaatctaacataattagaggatggatttttgggactacgcttgacagtaagacccaatcactcttgatcgtgaacaatgcaaccaatgtagtaattaaggtttgcgagtttcaattttgtaatgatccatttttgggggtttattaccacaaaaacaataaatcctggatggaatccgaattcagagtgtatagcagcgctaacaattgcacatttgagtacgtgtcacaaccttttcttatggatcttgagggcaagcaagggaacttcaaaaatttgagggagttcgttttcaagaacatcgacggatactttaagatctattctaaacacacccccattaacttggtgcgagatttgcctcaaggcttctctgcacttgaaccgttggtggatcttcccattggcattaatattactcggttccagactttgttggcactgcatcgctcctatctcacgcccggagacagttcatctggatggactgcgggggctgccgcgtattacgtgggatacctgcagccgcgcacatttcttcttaaatacaacgagaacgggaccatcacagatgcagtggattgcgctcttgaccccctctccgaaacaaaatgtacgctcaagtctttcactgtagagaaagggatttatcagacatccaatttccgagtccagccaacagagagtatagtgcggttccctaacatcacaaatctttgtccgttcggggaagttttcaacgctacacgcttcgcaagtgtatacgcttggaatagaaagaggatctctaattgtgtggcagattactctgtgctctacaattccgcatctttctcaaccttcaagtgttacggagtttcacctacgaagctgaacgacctttgctttactaatgtatatgcagatagttttgtcatcaggggcgatgaagttcgacagatagcgcccggccagacaggaaagatcgcggactacaattataaactccctgatgatttcaccgggtgcgtgatcgcgtggaactctaataacctcgactccaaagtaggcggtaactacaattacttgtatcgattgtttagaaaatcaaaccttaaaccgttcgagcgggatatctctaccgagatataccaagcaggctctacaccgtgcaatggagtcgaaggttttaactgctacttccccttgcaatcatacgggtttcagcctaccaacggtgtaggataccagccttacagggttgtggtactttcatttgagctcctgcacgctcccgcaactgtctgtgggcccaaaaagagcactaaccttgttaaaaataaatgcgtcaactttaacttcaatggcctcactggcaccggcgtgctcactgaaagcaataagaaattccttccttttcagcagtttgggcgagacatagcggataccacggacgcagtacgggatcctcaaacccttgaaatccttgacataacgccttgctcttttgggggagtaagcgtaatcacgcctggaaccaacacctccaatcaggttgctgtgctgtaccaggatgtaaactgcaccgaggtaccggtagccattcacgcggatcagctgactcccacatggcgagtgtattctacaggtagtaatgtgtttcagacccgagcagggtgtttgataggggcggagcacgtcaacaactcatacgagtgcgatatacccattggggctggtatatgtgcatcctaccagacgcagacgaactctcctgccgccgctgcgtctgttgcatctcaatcaattattgcatacactatgtcactgggggctgagaattcagtagcctactctaacaacagcatcgcgattcccactaacttcacaattagtgtgactaccgagatcctgccagtatccatgactaaaactagcgtagattgtactatgtacatctgcggcgattcaactgagtgttcaaacctcctcttgcaatacgggtcattttgtacccaattgaatcgagctctgaccggcatcgcggtcgaacaggacaaaaatactcaagaggtatttgcccaggtgaagcagatttacaaaacaccccctatcaaggatttcgggggcttcaacttcagccagatactgccagacccctcaaaaccgagcaagcgctccttcatagaagatcttcttttcaacaaagttaccctcgcggatgctggtttcattaaacagtatggggactgtctcggcgacattgctgctagagacctcatctgcgcgcaaaagttcaatggacttacggtcctgccccctctcctcactgatgaaatgattgctcaatatacgtccgcgttgttggcgggaactataaccagtgggtggacgttcggcgctggcgccgcgcttcaaatcccatttgcgatgcaaatggcgtatcgcttcaacggcatcggagtaactcaaaacgttctgtacgaaaatcaaaaactcattgcgaaccagtttaattcagcgatcggtaaaatccaggacagcctgagctccacagcgagtgcactcgggaagctccaggatgtggtaaatcagaacgctcaagcgttgaacacactcgtcaagcagctgtcaagtaactttggcgcgatttcatctgtattgaatgacattctctctcgccttgataaggtggaagccgaagtccagattgatcgcctgattactgggcggcttcagtccctccaaacatacgtcactcagcaacttattagagccgccgaaattagggcaagtgcgaatctggccgcgacaaaaatgtctgaatgtgtgctggggcagagcaagagagtcgatttttgcggtaaggggtatcaccttatgtcttttcctcagtctgcccctcacggagtagtgtttctccacgttacgtatgtcccagcccaagagaaaaactttaccactgcgccggctatttgtcatgacggtaaagcacactttccacgcgaaggtgtgttcgtctccaacggcacccactggtttgtaacgcagaggaacttctacgagcctcagataattaccacggacaacacgttcgtctcaggtaactgcgacgtcgtaattggtattgtaaataacaccgtgtacgacccgctccagccggagctggactccttcaaagaggagcttgacaagtattttaagaatcacacttcaccggatgtagacctgggggatatttccggcataaacgcttccgtggttaacatacagaaagagatagatcgactgaacgaggtagcgaaaaacttgaatgagtctttgatagacctgcaagaattgggaaaatatgaacaatatattaagtggccctggtatatttggcttggtttcatagccggtttgattgccatcgtcatggtaactataatgctttgttgcatgacaagttgctgctcttgcctcaaagggtgctgctcctgtggaagttgttgcaagttcgatgaggatgattctgagccagtgcttaagggtgtcaaactgcattatacgaggtcaattgctatgacgtttcttgcggttggaggagttttgctcttcctttcggtcaacgtccatgct

SEQ ID NO: 95 - protein, 1354 aa, Artificial Sequence
  fragment of construct 1
DQGCAINFGQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYTRSIAMTFLAVGGVLLFLSVNVHADQGCAINFGKRELKCGDGIFIFRDSDDWLNKYSYYPEDPVKLASIVKASFEEGKCGLNSVDS

SEQ ID NO: 96 - protein, 1292 aa, Artificial Sequence
  fragment of construct 2
DQGCAINFGQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPAAAASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYTRSIAMTFLAVGGVLLFLSVNVHA

SEQ ID NO: 97 - DNA, 3774 bp, Artificial Sequence
  fragment of construct 1
gtgaatcttaccacccgaacccagcttcctcccgcctacacgaattcattcacgcggggtgtttattacccggataaagttttccggtccagtgtcctgcattcaacccaagacctctttctgccatttttttctaacgtgacgtggttccatgctatccatgtaagcggaaccaacggaaccaaacggttcgataatccggttctcccattcaacgatggggtttacttcgcatctacagaaaaatctaacataattagaggatggatttttgggactacgcttgacagtaagacccaatcactcttgatcgtgaacaatgcaaccaatgtagtaattaaggtttgcgagtttcaattttgtaatgatccatttttgggggtttattaccacaaaaacaataaatcctggatggaatccgaattcagagtgtatagcagcgctaacaattgcacatttgagtacgtgtcacaaccttttcttatggatcttgagggcaagcaagggaacttcaaaaatttgagggagttcgttttcaagaacatcgacggatactttaagatctattctaaacacacccccattaacttggtgcgagatttgcctcaaggcttctctgcacttgaaccgttggtggatcttcccattggcattaatattactcggttccagactttgttggcactgcatcgctcctatctcacgcccggagacagttcatctggatggactgcgggggctgccgcgtattacgtgggatacctgcagccgcgcacatttcttcttaaatacaacgagaacgggaccatcacagatgcagtggattgcgctcttgaccccctctccgaaacaaaatgtacgctcaagtctttcactgtagagaaagggatttatcagacatccaatttccgagtccagccaacagagagtatagtgcggttccctaacatcacaaatctttgtccgttcggggaagttttcaacgctacacgcttcgcaagtgtatacgcttggaatagaaagaggatctctaattgtgtggcagattactctgtgctctacaattccgcatctttctcaaccttcaagtgttacggagtttcacctacgaagctgaacgacctttgctttactaatgtatatgcagatagttttgtcatcaggggcgatgaagttcgacagatagcgcccggccagacaggaaagatcgcggactacaattataaactccctgatgatttcaccgggtgcgtgatcgcgtggaactctaataacctcgactccaaagtaggcggtaactacaattacttgtatcgattgtttagaaaatcaaaccttaaaccgttcgagcgggatatctctaccgagatataccaagcaggctctacaccgtgcaatggagtcgaaggttttaactgctacttccccttgcaatcatacgggtttcagcctaccaacggtgtaggataccagccttacagggttgtggtactttcatttgagctcctgcacgctcccgcaactgtctgtgggcccaaaaagagcactaaccttgttaaaaataaatgcgtcaactttaacttcaatggcctcactggcaccggcgtgctcactgaaagcaataagaaattccttccttttcagcagtttgggcgagacatagcggataccacggacgcagtacgggatcctcaaacccttgaaatccttgacataacgccttgctcttttgggggagtaagcgtaatcacgcctggaaccaacacctccaatcaggttgctgtgctgtaccaggatgtaaactgcaccgaggtaccggtagccattcacgcggatcagctgactcccacatggcgagtgtattctacaggtagtaatgtgtttcagacccgagcagggtgtttgataggggcggagcacgtcaacaactcatacgagtgcgatatacccattggggctggtatatgtgcatcctaccagacgcagacgaactctcctgccgccgctgcgtctgttgcatctcaatcaattattgcatacactatgtcactgggggctgagaattcagtagcctactctaacaacagcatcgcgattcccactaacttcacaattagtgtgactaccgagatcctgccagtatccatgactaaaactagcgtagattgtactatgtacatctgcggcgattcaactgagtgttcaaacctcctcttgcaatacgggtcattttgtacccaattgaatcgagctctgaccggcatcgcggtcgaacaggacaaaaatactcaagaggtatttgcccaggtgaagcagatttacaaaacaccccctatcaaggatttcgggggcttcaacttcagccagatactgccagacccctcaaaaccgagcaagcgctccttcatagaagatcttcttttcaacaaagttaccctcgcggatgctggtttcattaaacagtatggggactgtctcggcgacattgctgctagagacctcatctgcgcgcaaaagttcaatggacttacggtcctgccccctctcctcactgatgaaatgattgctcaatatacgtccgcgttgttggcgggaactataaccagtgggtggacgttcggcgctggcgccgcgcttcaaatcccatttgcgatgcaaatggcgtatcgcttcaacggcatcggagtaactcaaaacgttctgtacgaaaatcaaaaactcattgcgaaccagtttaattcagcgatcggtaaaatccaggacagcctgagctccacagcgagtgcactcgggaagctccaggatgtggtaaatcagaacgctcaagcgttgaacacactcgtcaagcagctgtcaagtaactttggcgcgatttcatctgtattgaatgacattctctctcgccttgataaggtggaagccgaagtccagattgatcgcctgattactgggcggcttcagtccctccaaacatacgtcactcagcaacttattagagccgccgaaattagggcaagtgcgaatctggccgcgacaaaaatgtctgaatgtgtgctggggcagagcaagagagtcgatttttgcggtaaggggtatcaccttatgtcttttcctcagtctgcccctcacggagtagtgtttctccacgttacgtatgtcccagcccaagagaaaaactttaccactgcgccggctatttgtcatgacggtaaagcacactttccacgcgaaggtgtgttcgtctccaacggcacccactggtttgtaacgcagaggaacttctacgagcctcagataattaccacggacaacacgttcgtctcaggtaactgcgacgtcgtaattggtattgtaaataacaccgtgtacgacccgctccagccggagctggactccttcaaagaggagcttgacaagtattttaagaatcacacttcaccggatgtagacctgggggatatttccggcataaacgcttccgtggttaacatacagaaagagatagatcgactgaacgaggtagcgaaaaacttgaatgagtctttgatagacctgcaagaattgggaaaatatgaacaatatattaagtggccctggtatatttggcttggtttcatagccggtttgattgccatcgtcatggtaactataatgctttgttgcatgacaagttgctgctcttgcctcaaagggtgctgctcctgtggaagttgttgcaagttcgatgaggatgattctgagccagtgcttaagggtgtcaaactgcattatacg

SEQ ID NO: 98 - DNA, 4173 bp, Artificial Sequence
  Construct UK Spike variant
aaggtcatcatgggggcggtacttatatgggttggcatcaacacaagaaacatgacaatgtccatgagcatgatcttggtaggagtgatcatgatgtttttgtctctaggagttggcgccgaccagggctgcgcgataaatttcggtcaatgtgtgaatcttaccacccgaacccagcttcctcccgcctacacgaattcattcacgcggggtgtttattacccggataaagttttccggtccagtgtcctgcattcaacccaagacctctttctgccatttttttctaacgtgacgtggttccatgctatcagcggaaccaacggaaccaaacggttcgataatccggttctcccattcaacgatggggtttacttcgcatctacagaaaaatctaacataattagaggatggatttttgggactacgcttgacagtaagacccaatcactcttgatcgtgaacaatgcaaccaatgtagtaattaaggtttgcgagtttcaattttgtaatgatccatttttgggggtttaccacaaaaacaataaatcctggatggaatccgaattcagagtgtatagcagcgctaacaattgcacatttgagtacgtgtcacaaccttttcttatggatcttgagggcaagcaagggaacttcaaaaatttgagggagttcgttttcaagaacatcgacggatactttaagatctattctaaacacacccccattaacttggtgcgagatttgcctcaaggcttctctgcacttgaaccgttggtggatcttcccattggcattaatattactcggttccagactttgttggcactgcatcgctcctatctcacgcccggagacagttcatctggatggactgcgggggctgccgcgtattacgtgggatacctgcagccgcgcacatttcttcttaaatacaacgagaacgggaccatcacagatgcagtggattgcgctcttgaccccctctccgaaacaaaatgtacgctcaagtctttcactgtagagaaagggatttatcagacatccaatttccgagtccagccaacagagagtatagtgcggttccctaacatcacaaatctttgtccgttcggggaagttttcaacgctacacgcttcgcaagtgtatacgcttggaatagaaagaggatctctaattgtgtggcagattactctgtgctctacaattccgcatctttctcaaccttcaagtgttacggagtttcacctacgaagctgaacgacctttgctttactaatgtatatgcagatagttttgtcatcaggggcgatgaagttcgacagatagcgcccggccagacaggaaagatcgcggactacaattataaactccctgatgatttcaccgggtgcgtgatcgcgtggaactctaataacctcgactccaaagtaggcggtaactacaattacttgtatcgattgtttagaaaatcaaaccttaaaccgttcgagcgggatatctctaccgagatataccaagcaggctctacaccgtgcaatggagtcgaaggttttaactgctacttccccttgcaatcatacgggtttcagcctacctacggtgtaggataccagccttacagggttgtggtactttcatttgagctcctgcacgctcccgcaactgtctgtgggcccaaaaagagcactaaccttgttaaaaataaatgcgtcaactttaacttcaatggcctcactggcaccggcgtgctcactgaaagcaataagaaattccttccttttcagcagtttgggcgagacatagatgataccacggacgcagtacgggatcctcaaacccttgaaatccttgacataacgccttgctcttttgggggagtaagcgtaatcacgcctggaaccaacacctccaatcaggttgctgtgctgtaccagggtgtaaactgcaccgaggtaccggtagccattcacgcggatcagctgactcccacatggcgagtgtattctacaggtagtaatgtgtttcagacccgagcagggtgtttgataggggcggagcacgtcaacaactcatacgagtgcgatatacccattggggctggtatatgtgcatcctaccagacgcagacgaactctcatgccgccgctgcgtctgttgcatctcaatcaattattgcatacactatgtcactgggggctgagaattcagtagcctactctaacaacagcatcgcgattcccattaacttcacaattagtgtgactaccgagatcctgccagtatccatgactaaaactagcgtagattgtactatgtacatctgcggcgattcaactgagtgttcaaacctcctcttgcaatacgggtcattttgtacccaattgaatcgagctctgaccggcatcgcggtcgaacaggacaaaaatactcaagaggtatttgcccaggtgaagcagatttacaaaacaccccctatcaaggatttcgggggcttcaacttcagccagatactgccagacccctcaaaaccgagcaagcgctccttcatagaagatcttcttttcaacaaagttaccctcgcggatgctggtttcattaaacagtatggggactgtctcggcgacattgctgctagagacctcatctgcgcgcaaaagttcaatggacttacggtcctgccccctctcctcactgatgaaatgattgctcaatatacgtccgcgttgttggcgggaactataaccagtgggtggacgttcggcgctggcgccgcgcttcaaatcccatttgcgatgcaaatggcgtatcgcttcaacggcatcggagtaactcaaaacgttctgtacgaaaatcaaaaactcattgcgaaccagtttaattcagcgatcggtaaaatccaggacagcctgagctccacagcgagtgcactcgggaagctccaggatgtggtaaatcagaacgctcaagcgttgaacacactcgtcaagcagctgtcaagtaactttggcgcgatttcatctgtattgaatgacattctcgctcgccttgataaggtggaagccgaagtccagattgatcgcctgattactgggcggcttcagtccctccaaacatacgtcactcagcaacttattagagccgccgaaattagggcaagtgcgaatctggccgcgacaaaaatgtctgaatgtgtgctggggcagagcaagagagtcgatttttgcggtaaggggtatcaccttatgtcttttcctcagtctgcccctcacggagtagtgtttctccacgttacgtatgtcccagcccaagagaaaaactttaccactgcgccggctatttgtcatgacggtaaagcacactttccacgcgaaggtgtgttcgtctccaacggcacccactggtttgtaacgcagaggaacttctacgagcctcagataattaccacgcacaacacgttcgtctcaggtaactgcgacgtcgtaattggtattgtaaataacaccgtgtacgacccgctccagccggagctggactccttcaaagaggagcttgacaagtattttaagaatcacacttcaccggatgtagacctgggggatatttccggcataaacgcttccgtggttaacatacagaaagagatagatcgactgaacgaggtagcgaaaaacttgaatgagtctttgatagacctgcaagaattgggaaaatatgaacaatatattaagtggccctggtatatttggcttggtttcatagccggtttgattgccatcgtcatggtaactataatgctttgttgcatgacaagttgctgctcttgcctcaaagggtgctgctcctgtggaagttgttgcaagttcgatgaggatgattctgagccagtgcttaagggtgtcaaactgcattatacgaggtcaattgctatgacgtttcttgcggttggaggagttttgctcttcctttcggtcaacgtccatgctgatcaaggatgcgccatcaactttggcaagagagagctcaagtgcggagatggtatcttcatatttagagactctgatgactggctgaacaagtactcatactatccagaagatcctgtgaagcttgcatcaatagtgaaagcctcttttgaagaagggaagtgtggcctaaattcagttgactcc

SEQ ID NO: 99 - protein, 1391 aa, Artificial Sequence
  Construct UK Spike variant
KVIMGAVLIWVGINTRNMTMSMSMILVGVIMMFLSLGVGADQGCAINFGQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHAAAASVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYTRSIAMTFLAVGGVLLFLSVNVHADQGCAINFGKRELKCGDGIFIFRDSDDWLNKYSYYPEDPVKLASIVKASFEEGKCGLNSVDS

SEQ ID NO: 100 - DNA, 4173 bp, Artificial Sequence
  Construct South Africa (SA) Spike variant
aaggtcatcatgggggcggtacttatatgggttggcatcaacacaagaaacatgacaatgtccatgagcatgatcttggtaggagtgatcatgatgtttttgtctctaggagttggcgccgaccagggctgcgcgataaatttcggtcaatgtgtgaatttcaccacccgaacccagcttcctcccgcctacacgaattcattcacgcggggtgtttattacccggataaagttttccggtccagtgtcctgcattcaacccaagacctctttctgccatttttttctaacgtgacgtggttccatgctatccatgtaagcggaaccaacggaaccaaacggttcgctaatccggttctcccattcaacgatggggtttacttcgcatctacagaaaaatctaacataattagaggatggatttttgggactacgcttgacagtaagacccaatcactcttgatcgtgaacaatgcaaccaatgtagtaattaaggtttgcgagtttcaattttgtaatgatccatttttgggggtttattaccacaaaaacaataaatcctggatggaatccgaattcagagtgtatagcagcgctaacaattgcacatttgagtacgtgtcacaaccttttcttatggatcttgagggcaagcaagggaacttcaaaaatttgagggagttcgttttcaagaacatcgacggatactttaagatctattctaaacacacccccattaacttggtgcgaggtttgcctcaaggcttctctgcacttgaaccgttggtggatcttcccattggcattaatattactcggttccagactttgcatatctcctatctcacgcccggagacagttcatctggatggactgcgggggctgccgcgtattacgtgggatacctgcagccgcgcacatttcttcttaaatacaacgagaacgggaccatcacagatgcagtggattgcgctcttgaccccctctccgaaacaaaatgtacgctcaagtctttcactgtagagaaagggatttatcagacatccaatttccgagtccagccaacagagagtatagtgcggttccctaacatcacaaatctttgtccgttcggggaagttttcaacgctacacgcttcgcaagtgtatacgcttggaatagaaagaggatctctaattgtgtggcagattactctgtgctctacaattccgcatctttctcaaccttcaagtgttacggagtttcacctacgaagctgaacgacctttgctttactaatgtatatgcagatagttttgtcatcaggggcgatgaagttcgacagatagcgcccggccagacaggaaacatcgcggactacaattataaactccctgatgatttcaccgggtgcgtgatcgcgtggaactctaataacctcgactccaaagtaggcggtaactacaattacttgtatcgattgtttagaaaatcaaaccttaaaccgttcgagcgggatatctctaccgagatataccaagcaggctctacaccgtgcaatggagtcaaaggttttaactgctacttccccttgcaatcatacgggtttcagcctacctacggtgtaggataccagccttacagggttgtggtactttcatttgagctcctgcacgctcccgcaactgtctgtgggcccaaaaagagcactaaccttgttaaaaataaatgcgtcaactttaacttcaatggcctcactggcaccggcgtgctcactgaaagcaataagaaattccttccttttcagcagtttgggcgagacatagcggataccacggacgcagtacgggatcctcaaacccttgaaatccttgacataacgccttgctcttttgggggagtaagcgtaatcacgcctggaaccaacacctccaatcaggttgctgtgctgtaccagggtgtaaactgcaccgaggtaccggtagccattcacgcggatcagctgactcccacatggcgagtgtattctacaggtagtaatgtgtttcagacccgagcagggtgtttgataggggcggagcacgtcaacaactcatacgagtgcgatatacccattggggctggtatatgtgcatcctaccagacgcagacgaactctcctgccgccgctgcgtctgttgcatctcaatcaattattgcatacactatgtcactgggggttgagaattcagtagcctactctaacaacagcatcgcgattcccactaacttcacaattagtgtgactaccgagatcctgccagtatccatgactaaaactagcgtagattgtactatgtacatctgcggcgattcaactgagtgttcaaacctcctcttgcaatacgggtcattttgtacccaattgaatcgagctctgaccggcatcgcggtcgaacaggacaaaaatactcaagaggtatttgcccaggtgaagcagatttacaaaacaccccctatcaaggatttcgggggcttcaacttcagccagatactgccagacccctcaaaaccgagcaagcgctccttcatagaagatcttcttttcaacaaagttaccctcgcggatgctggtttcattaaacagtatggggactgtctcggcgacattgctgctagagacctcatctgcgcgcaaaagttcaatggacttacggtcctgccccctctcctcactgatgaaatgattgctcaatatacgtccgcgttgttggcgggaactataaccagtgggtggacgttcggcgctggcgccgcgcttcaaatcccatttgcgatgcaaatggcgtatcgcttcaacggcatcggagtaactcaaaacgttctgtacgaaaatcaaaaactcattgcgaaccagtttaattcagcgatcggtaaaatccaggacagcctgagctccacagcgagtgcactcgggaagctccaggatgtggtaaatcagaacgctcaagcgttgaacacactcgtcaagcagctgtcaagtaactttggcgcgatttcatctgtattgaatgacattctctctcgccttgataaggtggaagccgaagtccagattgatcgcctgattactgggcggcttcagtccctccaaacatacgtcactcagcaacttattagagccgccgaaattagggcaagtgcgaatctggccgcgacaaaaatgtctgaatgtgtgctggggcagagcaagagagtcgatttttgcggtaaggggtatcaccttatgtcttttcctcagtctgcccctcacggagtagtgtttctccacgttacgtatgtcccagcccaagagaaaaactttaccactgcgccggctatttgtcatgacggtaaagcacactttccacgcgaaggtgtgttcgtctccaacggcacccactggtttgtaacgcagaggaacttctacgagcctcagataattaccacggacaacacgttcgtctcaggtaactgcgacgtcgtaattggtattgtaaataacaccgtgtacgacccgctccagccggagctggactccttcaaagaggagcttgacaagtattttaagaatcacacttcaccggatgtagacctgggggatatttccggcataaacgcttccgtggttaacatacagaaagagatagatcgactgaacgaggtagcgaaaaacttgaatgagtctttgatagacctgcaagaattgggaaaatatgaacaatatattaagtggccctggtatatttggcttggtttcatagccggtttgattgccatcgtcatggtaactataatgctttgttgcatgacaagttgctgctcttgcctcaaagggtgctgctcctgtggaagttgttgcaagttcgatgaggatgattctgagccagtgcttaagggtgtcaaactgcattatacgaggtcaattgctatgacgtttcttgcggttggaggagttttgctcttcctttcggtcaacgtccatgctgatcaaggatgcgccatcaactttggcaagagagagctcaagtgcggagatggtatcttcatatttagagactctgatgactggctgaacaagtactcatactatccagaagatcctgtgaagcttgcatcaatagtgaaagcctcttttgaagaagggaagtgtggcctaaattcagttgactcc

SEQ ID NO: 101 - protein, 1391 aa, Artificial Sequence
  Construct South Africa (SA) Spike variant
KVIMGAVLIWVGINTRNMTMSMSMILVGVIMMFLSLGVGADQGCAINFGQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPAAAASVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYTRSIAMTFLAVGGVLLFLSVNVHADQGCAINFGKRELKCGDGIFIFRDSDDWLNKYSYYPEDPVKLASIVKASFEEGKCGLNSVDS

SEQ ID NO: 102 - DNA, 4182 bp, Artificial Sequence
  Construct Brazilian-Japanese (BR) Spike variant
aaggtcatcatgggggcggtacttatatgggttggcatcaacacaagaaacatgacaatgtccatgagcatgatcttggtaggagtgatcatgatgtttttgtctctaggagttggcgccgaccagggctgcgcgataaatttcggtcaatgtgtgaatttcaccaaccgaacccagcttccttccgcctacacgaattcattcacgcggggtgtttattacccggataaagttttccggtccagtgtcctgcattcaacccaagacctctttctgccatttttttctaacgtgacgtggttccatgctatccatgtaagcggaaccaacggaaccaaacggttcgataatccggttctcccattcaacgatggggtttacttcgcatctacagaaaaatctaacataattagaggatggatttttgggactacgcttgacagtaagacccaatcactcttgatcgtgaacaatgcaaccaatgtagtaattaaggtttgcgagtttcaattttgtaattatccatttttgggggtttattaccacaaaaacaataaatcctggatggaatccgaattcagagtgtatagcagcgctaacaattgcacatttgagtacgtgtcacaaccttttcttatggatcttgagggcaagcaagggaacttcaaaaatttgtcggagttcgttttcaagaacatcgacggatactttaagatctattctaaacacacccccattaacttggtgcgagatttgcctcaaggcttctctgcacttgaaccgttggtggatcttcccattggcattaatattactcggttccagactttgttggcactgcatcgctcctatctcacgcccggagacagttcatctggatggactgcgggggctgccgcgtattacgtgggatacctgcagccgcgcacatttcttcttaaatacaacgagaacgggaccatcacagatgcagtggattgcgctcttgaccccctctccgaaacaaaatgtacgctcaagtctttcactgtagagaaagggatttatcagacatccaatttccgagtccagccaacagagagtatagtgcggttccctaacatcacaaatctttgtccgttcggggaagttttcaacgctacacgcttcgcaagtgtatacgcttggaatagaaagaggatctctaattgtgtggcagattactctgtgctctacaattccgcatctttctcaaccttcaagtgttacggagtttcacctacgaagctgaacgacctttgctttactaatgtatatgcagatagttttgtcatcaggggcgatgaagttcgacagatagcgcccggccagacaggaacgatcgcggactacaattataaactccctgatgatttcaccgggtgcgtgatcgcgtggaactctaataacctcgactccaaagtaggcggtaactacaattacttgtatcgattgtttagaaaatcaaaccttaaaccgttcgagcgggatatctctaccgagatataccaagcaggctctacaccgtgcaatggagtcaaaggttttaactgctacttccccttgcaatcatacgggtttcagcctacctacggtgtaggataccagccttacagggttgtggtactttcatttgagctcctgcacgctcccgcaactgtctgtgggcccaaaaagagcactaaccttgttaaaaataaatgcgtcaactttaacttcaatggcctcactggcaccggcgtgctcactgaaagcaataagaaattccttccttttcagcagtttgggcgagacatagcggataccacggacgcagtacgggatcctcaaacccttgaaatccttgacataacgccttgctcttttgggggagtaagcgtaatcacgcctggaaccaacacctccaatcaggttgctgtgctgtaccagggtgtaaactgcaccgaggtaccggtagccattcacgcggatcagctgactcccacatggcgagtgtattctacaggtagtaatgtgtttcagacccgagcagggtgtttgataggggcggagtacgtcaacaactcatacgagtgcgatatacccattggggctggtatatgtgcatcctaccagacgcagacgaactctcctgccgccgctgcgtctgttgcatctcaatcaattattgcatacactatgtcactgggggctgagaattcagtagcctactctaacaacagcatcgcgattcccactaacttcacaattagtgtgactaccgagatcctgccagtatccatgactaaaactagcgtagattgtactatgtacatctgcggcgattcaactgagtgttcaaacctcctcttgcaatacgggtcattttgtacccaattgaatcgagctctgaccggcatcgcggtcgaacaggacaaaaatactcaagaggtatttgcccaggtgaagcagatttacaaaacaccccctatcaaggatttcgggggcttcaacttcagccagatactgccagacccctcaaaaccgagcaagcgctccttcatagaagatcttcttttcaacaaagttaccctcgcggatgctggtttcattaaacagtatggggactgtctcggcgacattgctgctagagacctcatctgcgcgcaaaagttcaatggacttacggtcctgccccctctcctcactgatgaaatgattgctcaatatacgtccgcgttgttggcgggaactataaccagtgggtggacgttcggcgctggcgccgcgcttcaaatcccatttgcgatgcaaatggcgtatcgcttcaacggcatcggagtaactcaaaacgttctgtacgaaaatcaaaaactcattgcgaaccagtttaattcagcgatcggtaaaatccaggacagcctgagctccacagcgagtgcactcgggaagctccaggatgtggtaaatcagaacgctcaagcgttgaacacactcgtcaagcagctgtcaagtaactttggcgcgatttcatctgtattgaatgacattctctctcgccttgataaggtggaagccgaagtccagattgatcgcctgattactgggcggcttcagtccctccaaacatacgtcactcagcaacttattagagccgccgaaattagggcaagtgcgaatctggccgcgataaaaatgtctgaatgtgtgctggggcagagcaagagagtcgatttttgcggtaaggggtatcaccttatgtcttttcctcagtctgcccctcacggagtagtgtttctccacgttacgtatgtcccagcccaagagaaaaactttaccactgcgccggctatttgtcatgacggtaaagcacactttccacgcgaaggtgtgttcgtctccaacggcacccactggtttgtaacgcagaggaacttctacgagcctcagataattaccacggacaacacgttcgtctcaggtaactgcgacgtcgtaattggtattgtaaataacaccgtgtacgacccgctccagccggagctggactccttcaaagaggagcttgacaagtattttaagaatcacacttcaccggatgtagacctgggggatatttccggcataaacgcttccttcgttaacatacagaaagagatagatcgactgaacgaggtagcgaaaaacttgaatgagtctttgatagacctgcaagaattgggaaaatatgaacaatatattaagtggccctggtatatttggcttggtttcatagccggtttgattgccatcgtcatggtaactataatgctttgttgcatgacaagttgctgctcttgcctcaaagggtgctgctcctgtggaagttgttgcaagttcgatgaggatgattctgagccagtgcttaagggtgtcaaactgcattatacgaggtcaattgctatgacgtttcttgcggttggaggagttttgctcttcctttcggtcaacgtccatgctgatcaaggatgcgccatcaactttggcaagagagagctcaagtgcggagatggtatcttcatatttagagactctgatgactggctgaacaagtactcatactatccagaagatcctgtgaagcttgcatcaatagtgaaagcctcttttgaagaagggaagtgtggcctaaattcagttgactcc

SEQ ID NO: 103 - protein, 1394 aa, Artificial Sequence
  Construct Brazilian-Japanese (BR) Spike variant
KVIMGAVLIWVGINTRNMTMSMSMILVGVIMMFLSLGVGADQGCAINFGQCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSPAAAASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASFVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYTRSIAMTFLAVGGVLLFLSVNVHADQGCAINFGKRELKCGDGIFIFRDSDDWLNKYSYYPEDPVKLASIVKASFEEGKCGLNSVDS